(19) United States
(12) Patent Application Publication
(10) Pub. No.: US 2023/0134835 A1
(43) Pub. Date: May 4, 2023

(54) METHODS OF SYNTHESIZING CAROTENOIDS

(71) Applicant: L.E.A.F. HOLDINGS GROUP LLC, Gulph Mills, PA (US)

(72) Inventors: Clet NIYIKIZA, Gulph Mills, PA (US); Victor Mandla MOYO, Ringoes, NJ (US); Bolin GENG, Gulph Mills, PA (US)

(21) Appl. No.: 17/917,910

(22) PCT Filed: Apr. 9, 2021
(86) PCT No.: PCT/US2021/026683

§ 371 Date: Oct. 7, 2022

Related U.S. Application Data
(60) Provisional application No. 63/007,777, filed on Apr. 9, 2020.

(51) Int. Cl. C07C 51/41; C07C 45/67; C07C 67/343; A61K 45/06

(52) U.S. Cl. CPC C07C 51/412; C07C 45/67; C07C 67/343; A61K 45/06

(57) ABSTRACT

Provided herein novel processes for preparing carotenoids, substantially pure carotenoids (such as substantially pure trans crocetin diesters and substantially pure trans sodium crocetinate), pharmaceutical compositions, and related methods of treatment and uses. The provided compositions have uses in treating diseases, disorders and conditions associated with, but not limited to, infection, ARDS, endotoxemia, inflammation, sepsis, ischemia, hypoxia, shock, stroke, lung injury, wound healing, traumatic injury, reperfusion injury, cardiovascular disease, kidney disease, liver disease, inflammatory disease, metabolic disease, pulmonary disorders, blood related disorders and hyperproliferative diseases such as cancer. Methods of making, and using the aqueous solutions and pharmaceutical compositions are also provided.

Carotenoids are a class of natural lipid-soluble pigments found principally in plants where they function as accessory pigments and impart protection of tissue through their ability to quench singlet oxygen and free radical species. Carotenoids are known to have antioxidant properties and consequently, provide numerous beneficial health effects including reducing the potential risks of cardiovascular diseases, cancers, and slowing and/or reversing the degenerative effects of aging on various human physiological activities. However, carotenoids are typically very lipophilic compounds and the clinical use of many carotenoids is limited by their instability and low bioavailability.

Crocetin is a carotenoid with antioxidative properties that is sparingly soluble in water. Chemically, crocetin is a 20-carbon apocarotenoid molecule containing seven double bonds and a carboxylic acid group at each end. The administration of trans crocetin (free acid), and its salt sodium trans crocetinate in free form (e.g., unencapsulated) pharmaceutical formulations has been reported to offer promise in the treatment for conditions caused by hypoxia, ischemia, and other medical conditions. However, neither has demonstrated clinical therapeutic efficacy. This is partly due to the fact that formulations of trans crocetin and its sodium salt, sodium trans crocetinate, (TSC), have been to date limited by instability, low bioavailability and short half-life.

### BRIEF SUMMARY

WO2019213538A1 discloses various liposome formulations that are effective in improving bioavailability and stability of various carotenoids.

To facilitate developments of carotenoids, the present disclosure provides novel synthetic processes, which can be employed for large scale synthesis of various carotenoids. The present disclosure further provides substantially pure carotenoids (such as substantially pure trans crocetin diesters and substantially pure TSC) and pharmaceutical compositions comprising the same. The substantially pure carotenoids herein can be employed as active pharmaceutical ingredients in the aqueous compositions or liposome formulations as described in WO2019213538A1 to deliver an effective amount of carotenoids to subjects in need. The provided compositions have uses in treating diseases and disorders and conditions associated with, but not limited to, infection, inflammation, sepsis, ischemia, hypoxia, shock, stroke, injury, cardiovascular disease, renal disease, liver disease, inflammatory disease, metabolic disease, pulmonary disease, neurodegenerative disease, disease of the immune system, and hyperproliferative diseases such as cancer. Methods of making, delivering, and using the aqueous solutions and pharmaceutical compositions are also provided, as are kits containing the compositions.

Carotenoid synthesis typically employs an olefin formation step, such as a Wittig reaction, to introduce a terminal ester/acid functional group at one or both ends of the molecule. See e.g., U.S. Pat. Nos. 8,030,350 and 8,269,027. The present disclosure is based in part on the unexpected discovery that the solvent system used in the olefin formation step can have a significant impact on the efficiency of this process. As shown in the Examples section, in one example, the use of acetonitrile and toluene as solvent system greatly improved the efficiency of separating the desired isomer. Because of this improvement, such olefin forming step can be easily scaled up to meet the requirements of commercial manufacturing. This ultimately leads to an overall improved process for the synthesis of carotenoids and higher purity of the carotenoid products.

In some embodiments, the disclosure provides:
[1] a method of preparing a carotenoid having Formula I:

Q-Y-Polyene Carotenoid-Y-Q, (I)
wherein,
the Polyene Carotenoid comprises
   (a) 3, 4, 5, 6, 7, 8, 9, 10, 3-5, 6-8, 9-10, or more than 9, conjugated double bonds, and
   (b) methyl or low alkyl (C2-C3) substitutions,
each Y is independently a bond,

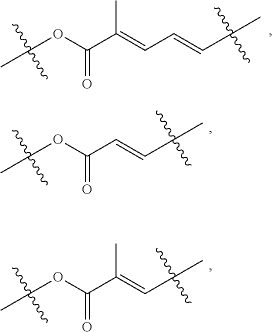

or a geometric isomer thereof,
provided that at least one Y has a —C(O)—O— functional group, and
   each Q is independently an ionizable group, hydrogen, an optionally substituted C₁₋₄ alkyl, or a counterion,
   the method comprising:
   a) reacting an aldehyde of Formula II with an olefin forming agent to form an ester (e.g., a C₁₋₄ alkyl ester, such as ethyl ester):

Q²-Y²-Polyene Carotenoid-Y²-Q², (II)
wherein at least one of Q²-Y² is HC(O)—, and when only one of Q²-Y² is HC(O)—, the other Q²-Y² is the same as Y-Q- in Formula I,
wherein the olefin forming agent is capable of reacting with the aldehyde to form a structural moiety of

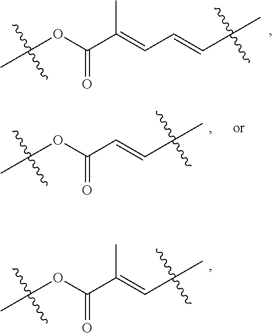

or a geometric isomer thereof,
and
b) optionally, when at least one Q is hydrogen or a counterion, hydrolyzing the ester to form the carotenoid of Formula I,
[1] the method of [1], wherein both Y are independently selected from

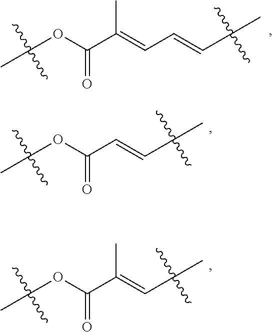

or a geometric isomer thereof,
[3] the method of [1] or [2], wherein both Y are the same,
[4] the method of any one of [1]-[3], wherein both Q are independently selected from hydrogen, an optionally substituted C₁₋₄ alkyl, or a counterion,
[5] the method of any one of [1]-[4], wherein both Q are the same,
[6] the method of [5], wherein both Q are C₁₋₄ alkyl,
[7] the method of [5], wherein both Q are hydrogen,
[8] the method of [5], wherein both Q are monovalent counterion, e.g., alkali metal ion, such as Li⁺, Na⁺, or K⁺,
[9] the method of any one of [1]-[8], wherein the Polyene Carotenoid is a hydrocarbon group having a total number of 6-36 carbons, such as 8, 10, 12, 14, 16, 18, or 20 carbons,
[10] the method of any one of [1]-[9], wherein the olefin forming agent is a phosphine based olefin forming agent,
[11] the method of any one of [1]-[10], wherein the olefin forming agent has the Formula III-1,

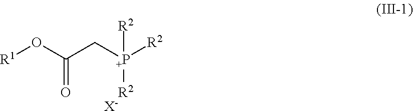

wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
[12] the method of any one of [1]-[11], wherein the olefin forming agent has the Formula of III-1-A:

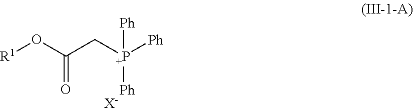

wherein R¹ is an optionally substituted C₁₋₄ alkyl, and X⁻ is a counterion, e.g., a halide such as chloride or bromide,
[13] the method of any one of [1]-[10], wherein the olefin forming agent has the Formula III-2,

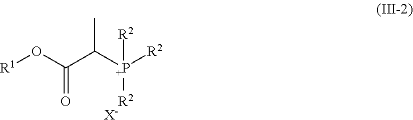

wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
[14] the method of any one of [1]-[10] and [13], wherein the olefin forming agent has the Formula of III-2-A:

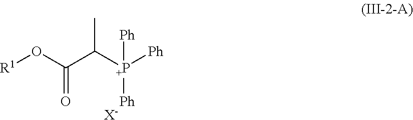

wherein R¹ is an optionally substituted C₁₋₄ alkyl, and X⁻ is a counterion, e.g., a halide such as chloride or bromide,
[15] the method of any one of [1]-[10], wherein the olefin forming agent has the Formula III-3,

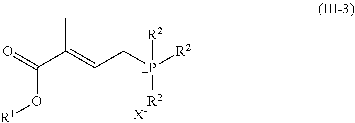

wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
[16] the method of any one of [1]-[10] and [15], wherein the olefin forming agent has the Formula III-3-A:

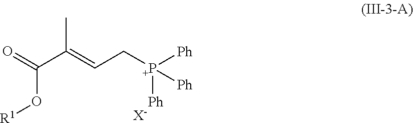

wherein R¹ is an optionally substituted C₁₋₄ alkyl, and X⁻ is a counterion, e.g., a halide such as chloride or bromide,
[17] the method of any one of [11]-[16], wherein R¹ is not methyl,
[18] the method of any one of [11]-[17], wherein R¹ is ethyl,
[19] the method of any one of [1]-[18], wherein the olefin forming reaction is carried out in a non-ether solvent in the presence of a base such as an inorganic base,
[20] the method of any one of [1]-[19], wherein the olefin forming reaction is carried out in a solvent system comprising acetonitrile, e.g., comprising acetonitrile and toluene,
[21] the method of [19] or [20], wherein the base is a carbonate base, e.g., sodium carbonate, potassium carbonate, sodium bicarbonate, potassium bicarbonate, etc.,
[22] the method of any one of [1]-[21], wherein the carotenoid of formula I has the Formula I-1:

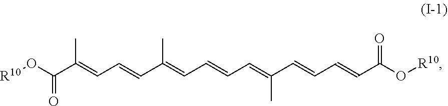

or a geometric isomer thereof, or a combination thereof, wherein each R¹⁰ is independently selected from hydrogen, a counterion (e.g., a monovalent counterion or a divalent counterion), and an optionally substituted C₁₋₄ alkyl, preferably, both R¹⁰ are the same,
[23] the method of [22], wherein both R¹⁰ are C₁₋₄ alkyl, preferably, ethyl,
[24] the method of [23], wherein both R¹⁰ are a monovalent counterion, preferably, an alkali metal ion, such as Li⁺, Na⁺, or K⁺,
[25] the method of [24], wherein both R¹⁰ are hydrogen,
[26] the method of any one of [22]-[25], wherein the aldehyde of formula II has the Formula II-1:

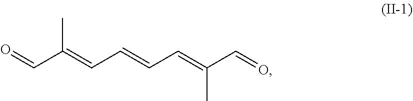

or a geometric isomer thereof, or a combination thereof,
[27] the method of [26], wherein the aldehyde of formula II used for reacting with the olefin forming agent is a substantially pure form of Formula II-1:

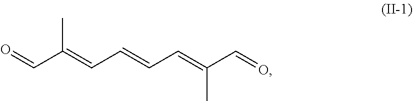

which includes less than 10% of other geometric isomers by HPLC area percentage with detection wavelength at 330 nm, preferably, less than 5% such as less than 3%, or less than 2% of other geometric isomers,
[28] the method of any one of [1]-[21], wherein the carotenoid of formula I has the Formula I-2:

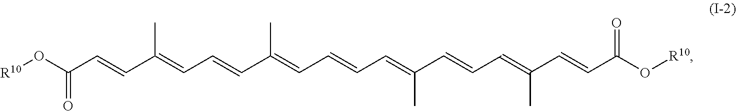

or a geometric isomer thereof, or a combination thereof, wherein each R¹⁰ is independently selected from hydrogen, a counterion (e.g., a monovalent counterion or a divalent counterion), and an optionally substituted C₁₋₄ alkyl, preferably, both R¹⁰ are the same,
[29] the method of [28], wherein both R¹⁰ are C₁₋₄ alkyl, preferably, ethyl,
[30] the method of [28], wherein both R¹⁰ are a monovalent counterion, preferably, an alkali metal ion, such as Li⁺, Na⁺, or K⁺,
[31] the method of [28], wherein both R¹⁰ are hydrogen,
[32] the method of any one of [28]-[31], wherein the aldehyde of formula II has the Formula II-2:

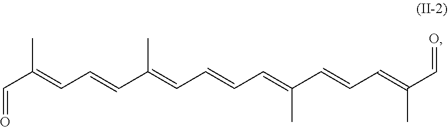 [SMILES: C\C(C=O)=C/C=C/C(C)=C/C=C/C=C(C)/C=C/C=C(\C)C=O]

or a geometric isomer thereof, or a combination thereof,
[33] the method of [32], wherein the aldehyde of Formula II used for reacting with the olefin forming agent is a substantially pure form of Formula II-2,

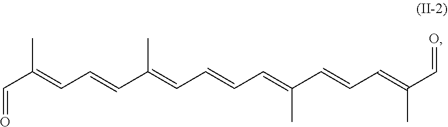 [SMILES: C\C(C=O)=C/C=C/C(C)=C/C=C/C=C(C)/C=C/C=C(\C)C=O]

which includes less than 10% of other geometric isomers by HPLC area percentage with detection wavelength at 254 nm and 420 nm, preferably, less than 5% such as less than 3%, or less than 2% of other geometric isomers,
[34] a method of preparing a crocetin having formula I-1:

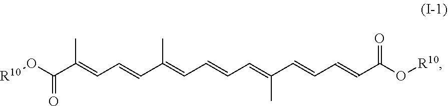

which is in a substantially pure form, e.g., with an applicable purity profile described herein, such as having less than 3% of other geometric isomers by HPLC area percentage with detection wavelength at 254 nm and 420 nm, preferably, less than 2% such as less than 1.5%, or less than 1% of other geometric isomers, wherein each R¹⁰ is independently selected from hydrogen, a counterion (e.g., a monovalent counterion or a divalent counterion), and an optionally substituted C₁₋₄ alkyl, the method comprising:
a) reacting a dialdehyde of Formula II-1:

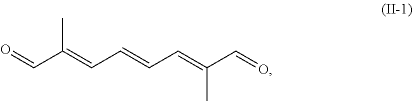

wherein the dialdehyde contains less than 10% of other geometric isomers by HPLC area percentage with detection wavelength at 330 nm, preferably, less than 5% such as less than 3%, or less than 2% of other geometric isomers,
with an olefin forming agent of Formula III-3,

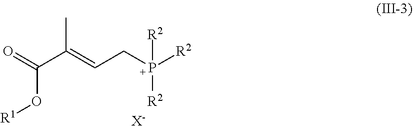

wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
to form an intermediate compound of Formula IV-1,

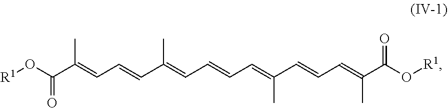

b) isolating the intermediate compound of Formula IV-1 in a substantially pure form, e.g., with an applicable purity profile described herein, such as having less than 3% of other geometric isomers by HPLC area percentage with detection wavelength at 254 nm and 420 nm, preferably, less than 2% such as less than 1.5%, or less than 1% of other geometric isomers, and
c) optionally hydrolyzing the intermediate compound of Formula IV-1 into the compound of Formula I-1 when at least one R¹⁰ is hydrogen or a counterion,
[35] the method of [34], wherein both R¹ are the same,
[36] the method of [35], wherein R¹ is not methyl,
[37] the method of [35], wherein R¹ is ethyl,
[38] the method of any one of [34]-[37], wherein both R¹⁰ are the same,
[39] the method of [38], wherein R¹⁰ is ethyl,
[40] the method of [38], wherein R¹⁰ is hydrogen,
[41] the method of [38], wherein R¹⁰ is a monovalent counterion, preferably, an alkali metal ion, such as Li⁺, Na⁺, or K⁺,
[42] the method of any one of [34]-[41], wherein the olefin forming agent is a compound of Formula III-3-A,

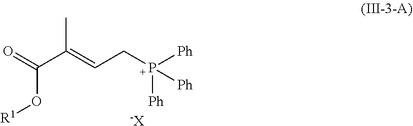

wherein X⁻ is a counterion, e.g., a halide such as chloride or bromide,
[43] the method of any one of [34]-[42], wherein the reacting of the dialdehyde and the olefin forming agent is carried out in a non-ether solvent in the presence of a base such as an inorganic base,
[44] the method of [43], wherein the reacting of the dialdehyde and the olefin forming agent is carried out in a solvent comprising acetonitrile, such as comprising acetonitrile and toluene,
[45] the method of [43] or [44], wherein the base is a carbonate base, e.g., sodium carbonate, potassium carbonate, sodium bicarbonate, potassium bicarbonate, etc.,
[46] the method of any one of [43]-[45], wherein the reacting of the dialdehyde and the olefin forming agent is carried out under heat, e.g., with a reaction temperature ranging from about 50° C.-100° C., or the reaction temperature can be about 45° C. to about 65° C., such as about 60° C., for a first period of time ranging from about 1 hour to about 6 hours, such as about 2, 3, 4, or 5 hours; and subsequently, the reaction temperature can be about 70° C. to about 100° C., such as about 80° C., for a second period of time of ranging from about 10 hours to about 48 hours, such as about 12, 14, 16, 18, 20, or 24 hours,
[47] the method of any one of [34]-[46], wherein isolating the intermediate compound of Formula IV-1 comprises cooling the reaction mixture, filtering the reaction mixture, washing the filtered solids with acetonitrile and water, and removing residue phosphine oxide through slurring the washed solids in an alcoholic solvent (e.g., methanol, ethanol, etc.),
[48] the method of any one of [34]-[47], wherein the intermediate compound of Formula IV-1 is isolated in a purity of greater than 95%, preferably, greater than 97%, greater than 98%, by HPLC area percentage with detection wavelength at 254 nm and 420 nm,
[49] the method of any one of [34]-[48], wherein the dialdehyde of Formula II-1 is prepared by a process comprising isomerizing one or more geometric isomers of Formula II-1, e.g., under an acidic condition, such as with a catalytic amount of phenylsulfinic acid (e.g., generated from sodium phenylsulfinate and HCl), or other acids with similar pKa,
[50] the method of any one of [34]-[49], further comprising hydrolyzing the intermediate compound of Formula IV-1, e.g., with an alkali hydroxide, such as LiOH, NaOH, or KOH, to provide the compound of Formula I-1, wherein one or both R¹⁰ is hydrogen or a counterion,
[51] the method of any one of [34]-[50], wherein both R¹⁰ are Na⁺,
[52] the method of [51], further comprising isolating the compound of Formula I-1 from the hydrolysis reaction mixture, e.g., through filtering the solid formed in the reaction mixture, washing the solid with water, washing the solid with an alcoholic solvent (e.g., ethanol), and drying the solid,
[53] the method of [52], wherein the compound of Formula I-1 is isolated in a substantially pure form, e.g., with an applicable purity profile described herein, such as having a purity of greater than 95%, preferably, greater than 97%, greater than 98%, greater than 99%, by HPLC area percentage with detection wavelength at 254 nm and 420 nm,
[54] the carotenoid of Formula I produced by the method of any of [1]-[33],
[55] the crocetin of Formula I-1 produced by the method of any of [34]-[53],
[56] a crocetin compound of Formula I-1, such as TSC, which is in a substantially pure form, e.g., with an applicable purity profile described herein, such as having a purity of greater than 95%, preferably, greater than 97%, greater than 98%, greater than 99%, by HPLC area percentage with detection wavelength at 254 nm and 420 nm,
[57] a method of preparing a pharmaceutical composition comprising formulating the carotenoid of Formula I of [54] or the crocetin of Formula I-1 of [55] or [56]
[58] the method of [57], comprising mixing the carotenoid of Formula I of [54] or the crocetin of Formula I-1 of [55] or [56] with one or more pharmaceutically acceptable excipient or carrier,
[59] the method of [58], wherein the one or more pharmaceutically acceptable excipient or carrier comprises a polyethylene glycol having a molecular weight of 200-700 Da, e.g., PEG-200, PEG-300, PEG-400, PEG-500, or PEG-600, etc.,
[60] the method of any of [57]-[59], further comprising
   a) mixing the carotenoid of Formula I of [54] or the crocetin of Formula I-1 of [55] or [56] with one or more pharmaceutically acceptable excipient or carrier with a liposomal solution containing liposomes comprising a weak acid salt of a multivalent metal; and
   b) maintaining the mixture of a) for a sufficient time to load the carotenoid or crocetin into liposomes;
[61] the method of [60], wherein the weak acid is an organic acid (e.g., an organic acid selected from acetic acid, gluconic acid, tartaric acid, glutamic acid, citric acid, formic acid, and glycinic acid);
[62] the method of [60] or [61], wherein the multivalent metal is a bivalent metal (e.g., a bivalent metal selected from Ca²⁺, Mg²⁺, Zn²⁺, Cu²⁺, Co²⁺, and Fe²⁺), or a trivalent metal such as Fe³⁺;
[63] the method of any of [60]-[62], wherein the weak acid is acetic acid and the bivalent metal is Ca²⁺ or Mg²⁺ (i.e., the weak acid salt of the bivalent metal is calcium acetate or magnesium acetate, respectively);
[64] the method of any one of [60]-[63], which further comprises the step of heating and cooling of the drug loading mixture;
[65] an aqueous solution comprising a liposome prepared according to the method of any one of [60]-[64];
[66] a pharmaceutical composition comprising a liposome prepared according to the method of any one of [60]-[64] or the aqueous solution of [65];
[67] a pharmaceutical composition comprising the carotenoid of Formula I of [54] or the crocetin of Formula I-1 of [55] or [56],
[68] a pharmaceutical composition produced by the method of any of [57]-[64],
[69] the pharmaceutical composition of any of [66]-[68], for use in the treatment or prevention of a disease or a condition in a subject;
[70] use of the pharmaceutical composition of any of [66]-[68], in the manufacture of a medicament for the treatment or prevention of a disease or condition in a subject;
[71] a method for treating or preventing a disease or condition in a subject needing such treatment or prevention, the method comprising administering an effective amount of the pharmaceutical composition of any of [66]-[68] to the subject;
[72] the pharmaceutical composition of [69], use of [70], or method of [71], wherein the disease or condition is characterized by ischemia (e.g., tissue hypoperfusion) or hypoxia;
[73] the pharmaceutical composition, use, or method, of [72], wherein the disease or condition characterized by ischemia or hypoxia is tissue hypoperfusion, ischemic-reperfusion injury, transient cerebral ischemia, cerebral ischemia-reperfusion, ischemic stroke, hemorrhagic stroke, traumatic brain injury, migraine (e.g., a chronic migraine or severe migraine disorder), gastrointestinal ischemia, kidney disease, pulmonary embolism, acute respiratory failure, neonatal respiratory distress syndrome, an obstetric emergency to reduce perinatal comorbidity (such as, pre/eclampsia and conditions that lead to cerebral palsy), myocardial infarction, acute limb or mesenteric ischemia, cardiac cirrhosis, chronic peripheral vascular disease, congestive heart failure, atherosclerotic stenosis, anemia, thrombosis, or embolism, wherein the subject has experienced a traumatic injury, or wherein the subject is undergoing or will undergo surgery;
[74] the pharmaceutical composition of [69], use of [70], or method of [71], wherein the disease or condition is a lung disease or a condition associated with a lung disease;
[75] the pharmaceutical composition, use, or method of [74], wherein the disease or condition is pulmonary fibrosis, pulmonary hemorrhage, lung injury, lung cancer, chronic obstructive pulmonary disease (COPD), or another respiratory disorder;
[76] the pharmaceutical composition of [69], use of [70], or method of [71], wherein the disease or condition is acute respiratory distress syndrome (ARDS);
[77] the pharmaceutical composition of [69], use of [70], or method of [71], wherein the disease or condition is a cardiovascular disease or a condition associated with a cardiovascular disease;
[78] the pharmaceutical composition, use, or method of [77], wherein the disease or condition is a coronary artery disease such as myocardial infarction, sudden cardiac death, cardiorespiratory arrest, hypertension, pulmonary arterial hypertension, atherosclerosis, Takayasu's arthritis, and post-cardiac arrest syndrome (PCAS), chronic venous insufficiency, heart disease, congestive heart failure, occlusive arterial disease, Raynaud's disease, peripheral vascular disease, or another vasculopathy such as Buerger's disease, or a chronic skin ulcer;
[79] the pharmaceutical composition of [69], use of [70], or method of [71], wherein the disease or condition is a heart attack or stroke, or a condition associated with a heart attack or stroke (e.g., an ischemic stroke or hemorrhagic stroke);
[80] the pharmaceutical composition of [69], use of [70], or method of [71], wherein the disease or condition is associated with nitric oxide deficiency (e.g., sickle cell disease, paroxysmal nocturnal hemoglobinuria (PNH), a hemolytic anemia, a thalassemia, another red blood cell disorder, a purpura such as thrombotic thrombocytic purpura (TTP), hemolytic uremic syndrome (HUS), idiopathic thrombocytopenia (ITP), another platelet disorder, a coagulation abnormality such as disseminated intravascular coagulopathy (DIC), purpura fulminans, heparin induced thrombocytopenia (HIT), hyperleukocytosis, and or hyper viscosity syndrome);
[81] the pharmaceutical composition of [69], use of [70], or method of [71], wherein the disease or condition is shock or a condition associated with shock (e.g., cardiogenic shock, hypovolemic shock, septic shock, neurogenic shock, and anaphylactic shock);
[82] the pharmaceutical composition of [69], use of [70], or method of [71], wherein the disease or condition is an infection or a condition associated with an infection (e.g., hypoxia, tissue hypoperfusion, ischemia, ARDS, and sepsis);
[83] the pharmaceutical composition, use, or method, of [82], wherein the disease or condition is associated with a bacterial infection (e.g., an P. aeruginosa infection, S. aureus infection (e.g., MRSA), or an enterococcal infection (e.g., VRE), such as hypoxia, tissue hypoperfusion, ischemia, and sepsis);
[84] the pharmaceutical composition, use, or method, of [82], wherein the disease or condition is associated with a viral infection (e.g., hypoxia, tissue hypoperfusion, ischemia, sepsis, and ARDS);
[85] the pharmaceutical composition, use, or method, of [82], wherein the disease or condition is associated with a coronavirus infection (e.g., COVID-19 and ARDS);
[86] the pharmaceutical composition, use, or method, of [82], wherein the disease or condition is associated with an Ebola, Dengue or Marburg infection (e.g., influenza, measles, and a viral hemorrhagic fever);
[87] the pharmaceutical composition, use, or method, of [82], wherein the disease or condition is associated with a fungal infection (e.g., a candidiasis infection such as invasive candidiasis);
[88] the pharmaceutical composition, use, or method, of [82], wherein the disease or condition is a parasitic infection or a condition associated with a parasitic infection such as malaria (e.g., cerebral malaria, severe anemia, acidosis, acute kidney failure, ischemia, tissue hypoperfusion and ARDS), Schistosomiasis, and human African trypanosomiasis;
[89] the pharmaceutical composition of [69], use of [70], or method of [71], wherein the disease or condition is endotoxemia or a condition associated with endotoxemia (e.g., low grade endotoxemic disease, or endotoxemia associated with a condition such as periodontal disease (e.g., periodontitis or inflammation of the gums), chronic alcoholism, chronic smoking, transplantation, neonatal necrotizing enterocolitis, or a neonatal ear infection);
[90] the pharmaceutical composition of [69], use of [70], or method of [71], wherein the disease or condition is bacteremia;
[91] the pharmaceutical composition of [69], use of [70], or method of [71], wherein the disease or condition is sepsis; or a condition associated with sepsis, or wherein the subject is at risk of developing sepsis or a condition associated with sepsis;
[92] the pharmaceutical composition of [69], use of [70], or method of [71], wherein the disease or condition is inflammation or a condition associated with inflammation;
[93] the pharmaceutical composition, use, or method of [92], wherein the disease or condition is systemic inflammation (e.g., systemic inflammatory response syndrome (SIRS)), low-grade inflammation, acute inflammation, or a chronic inflammatory disease;
[94] the pharmaceutical composition of [69], use of [70], or method of [71], wherein the disease or condition is an autoimmune disorder (e.g., psoriasis, cystic fibrosis, and rheumatoid arthritis);
[95] the pharmaceutical composition of [69], use of [70], or method of [71], wherein the disease or condition is sclerosis or a condition associated with sclerosis (e.g., systemic sclerosis);
[96] the pharmaceutical composition of [69], use of [70], or method of [71], wherein the disease or condition is inflammatory bowel disease or a condition associated with inflammatory bowel disease (e.g., Crohn's disease and ulcerative colitis);
[97] the pharmaceutical composition of [69], use of [70], or method of [71], wherein the disease or condition is a metabolic disease or a condition associated with a metabolic disease;
[98] the pharmaceutical composition of [69], use of [70], or method of [71], wherein the disease or condition is insulin resistance or a condition associated with insulin resistance;
[99] the pharmaceutical composition of [69], use of [70], or method of [71], wherein the disease or condition is diabetes or a condition associated with diabetes such as gangrene, diabetic necrosis, diabetic neuropathy, diabetic vascular disease (e.g., microvascular disease such as retinopathy and nephropathy, and diabetic ulcers);
[100] the pharmaceutical composition of [69], use of [70], or method of [71], wherein the disease or condition is type 2 diabetes or a condition associated with type 2 diabetes;
[101] the pharmaceutical composition of [69], use of [70], or method of [71], wherein the disease or condition is liver disease or a condition associated with a liver disease (e.g., cirrhosis, nonalcoholic fatty liver disease (NAFLD), non-alcoholic steatohepatitis (NASH); alcoholic liver disease, acute liver injury, and cirrhosis of the liver);
[102] the pharmaceutical composition of [69], use of [70], or method of [71], wherein the disease or condition is kidney disease or a condition associated with kidney disease (e.g., lipopolysaccharide medication or toxin induced acute kidney injury (AKI) and end stage kidney disease);
[103] use of the pharmaceutical composition of any of [66]-[68] in the manufacture of a medicament for increasing the delivery of oxygen to the cells in a subject;
[104] a method of increasing the delivery of oxygen to the cells and tissues in a subject, the method comprising administering an effective amount of the pharmaceutical composition of any of [66]-[68] to the subject;
[105] the use of [103] or method of [104], wherein the subject is in need of increased oxygen delivery;
[106] the use of [103] or method of [104], wherein the pharmaceutical composition is administered to increase the physical or mental performance of the subject;
[107] use of the pharmaceutical composition of any of [66]-[68] in the manufacture of a medicament for reducing systemic levels of LPS, endotoxin and/or another trigger of systemic inflammation in a subject;
[108] a method of reducing systemic levels of LPS, endotoxin and/or another trigger of systemic inflammation in a subject in need thereof, the method comprising administering an effective amount of the pharmaceutical composition any of [66]-[68] to the subject;
[109] the use or method according to any one of [69]-[108], wherein the age of the subject is 50 or older, 55 or older, 60 or older, 65 or older, 70 or older, 75 or older, or 80 or older;
[110] the use or method according to any one of [69]-[109], wherein the subject is immunocompromised;
[111] the use or method according to any one of [69]-[110], wherein the subject receives chemotherapy and/or is immune-suppressed (e.g., febrile neutropenic patients);
[112] the use or method according to any one of [69]-[111], wherein the subject is a burn victim;
[113] the use or method according to any one of [69]-[112], wherein the subject is critically ill;
[114] the use or method according to any one of [69]-[113], wherein the pharmaceutical composition is administered in combination therapy with another therapeutic agent,
[115] the use or method according to any one of [69]-[114], wherein subject is a human.

### BRIEF DESCRIPTION OF THE DRAWINGS/FIGURES

FIG. 1 shows a representative trace and integration results from an HPLC analysis of the C10 cis/trans dial obtained from hydrolysis of the C10-cis/trans diacetal, with the detection wavelength at 330 nm.

FIG. 2 shows a representative trace and integration results from an HPLC analysis of the C10 trans dial after the isomerization reaction, with the detection wavelength at 330 nm.

FIG. 3 shows a representative trace and integration results from an HPLC analysis of the C20 trans diester obtained from the Wittig reaction, with the detection wavelength at 254 nm or 420 nm.

FIG. 4 shows a representative trace and integration results from an HPLC analysis of trans sodium crocetinate (TSC) obtained from hydrolysis of the C20 trans diester, with the detection wavelength at 254 nm or 420 nm.

FIG. 5 shows a specification of TSC and test results of one batch of TSC produced by the process described herein.

### DETAILED DESCRIPTION

In various embodiments, the present disclosure provides novel processes for preparing carotenoids, substantially pure carotenoids (such as substantially pure trans crocetin diesters and substantially pure TSC), pharmaceutical compositions, and related methods of treatment and uses.

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Other features and advantages of the disclosed compositions and methods will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight, mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean +/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments, of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed compositions and methods also envisage the explicit exclusion of one or more of any of the group members in the disclosed compositions or methods.

As used herein, the term “alkyl” as used by itself or as part of another group refers to a straight- or branched-chain saturated aliphatic hydrocarbon. In one embodiment, the alkyl group is a C₁₋₄ alkyl group selected from methyl, ethyl, propyl (n-propyl), isopropyl, butyl (n-butyl), sec-butyl, tert-butyl, and iso-butyl. An optionally substituted C₁₋₄ alkyl group refers to the C₁₋₄ alkyl group as defined, optionally substituted with one or more permissible substituents.

“Cycloalkyl” as used by itself or as part of another group refers to a radical of a non-aromatic cyclic hydrocarbon group having from 3 to 10 ring carbon atoms (“C₃₋₁₀ cycloalkyl”) and zero heteroatoms in the non-aromatic ring system. The cycloalkyl group can be either monocyclic or contain a fused, bridged or spiro ring system such as a bicyclic system and can be saturated or can be partially unsaturated.

An “optionally substituted” group, such as an optionally substituted alkyl, cycloalkyl, optionally substituted phenyl, refers to the respective group that is unsubstituted or substituted. In general, the term “substituted”, whether preceded by the term “optionally” or not, means that at least one hydrogen present on a group (e.g., a carbon atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., as synthetic reagents or intermediates).

In some embodiments, the “optionally substituted” non-aromatic group herein can be unsubstituted or substituted with 1, 2, or 3 substituents independently selected from F, Cl, —OH, oxo (as applicable), C₁₋₄ alkyl, C₂₋₄ alkenyl, C₂₋₄ alkynyl, C₁₋₄ alkoxy, C₃₋₆cycloalkyl, C₃₋₆ cycloalkoxy, phenyl, 5 or 6 membered heteroaryl containing 1 or 2 ring heteroatoms independently selected from O, S, and N, 4-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aromatic group (including aryl and heteroaryl groups) herein can be unsubstituted or substituted with 1, 2, or 3 substituents independently selected from F, Cl, —OH, —CN, C₁₋₄ alkyl, C₂₋₄ alkenyl, C₂₋₄ alkynyl, C₁₋₄ alkoxy, C₃₋₆cycloalkyl, C₃₋₆ cycloalkoxy, phenyl, 5 or 6 membered heteroaryl containing 1 or 2 ring heteroatoms independently selected from O, S, and N, 4-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

As used herein, the term “olefin forming agent” refers to an agent that can react with a ketone or aldehyde to form an olefin bond (i.e., C═C double bond) under suitable conditions. In some embodiments, the olefin forming agent can be a Wittig reagent. In some embodiments, the olefin forming agent can be a phosphorous ylide, or a precursor thereof, e.g., a phosphonium salt that can form the phosphorus ylide under certain conditions (e.g., using a base). In some embodiments, the olefin forming agent comprises an organometallic reagent (e.g., an organozinc reagent, such as a Reformatsky reagent), wherein the ketone or aldehyde can be converted into an olefin by first forming an alcoholic intermediate with the organometallic reagent, which is followed by dehydration.

As used herein, the term “ylide” refers to a neutral dipolar molecule containing a formally negatively charged atom (usually a carbanion) directly attached to a heteroatom with a formal positive charge (usually nitrogen, phosphorus or sulfur). Thus, when the heteroatom is phosphine, the ylide is a “phosphorous ylide.” For example, methylenetriphenylphosphorane (Ph₃P═CH₂) is a phosphorous ylide.

The term “geometric isomer(s)” as used herein means isomer(s) of identical structure except with different configurations at the double bond(s) (i.e., E or Z isomer, or cis/trans isomer).

It is also meant to be understood that a specific embodiment of a variable moiety herein can be the same or different as another specific embodiment having the same identifier.

The term “liposome” refers to a closed vesicle having an internal phase (i.e., interior space (internal solution)) enclosed by lipid bilayer. A liposome can be a small single-membrane liposome such as a small unilamellar vesicle (SUV), large single-membrane liposome such as a large unilamellar vesicle (LUV), a still larger single-membrane liposome such as a giant unilamellar vesicle (GUV), a multilayer liposome having multiple concentric membranes (e.g., 2, 3, 4, 5, 6, 7, 8, 9, or 10), such as a multilamellar vesicle (MLV), or a liposome having multiple membranes that are irregular and not concentric such as a multivesicular vesicle (MVV). Liposomes and liposome formulations are well known in the art. Lipids which are capable of forming liposomes include all substances having fatty or fat-like properties. Lipids which can make up the lipids in the liposomes include without limitation, glycerides, glycerophospholipids, glycerophosphinolipids, glycerophos-phonolipids, sulfo-lipids, sphingolipids, phospholipids, isoprenolides, steroids, stearines, sterols, archeolipids, synthetic cationic lipids and carbohydrate containing lipids.

A “liposome composition” is a prepared composition comprising a liposome and the contents within the liposome, particularly including the lipids which form the liposome bilayer(s), compounds other than the lipids within the bi-layer(s) of the liposome, compounds within and associated with the aqueous interior(s) of the liposome, and compounds bound to or associated with the outer layer of the liposome. Thus, in addition to the lipids of the liposome, a liposome composition described herein suitably may include, but is not limited to, therapeutic agents, immunostimulating agents, vaccine antigens and adjuvants, excipients, carriers and buffering agents. In a preferred embodiment, such compounds are complementary to and/or are not significantly detrimental to the stability or AGP-incorporation efficiency of the liposome composition.

The term “counterion” refers to an anionic or cationic counterion. A “cationic counterion” is a positively charged atom or group associated with an anionic atom or group in order to maintain electronic neutrality. Exemplary cationic counterions include inorganic cations (e.g., metal cations (e.g., alkali metal cations, alkali earth metal cations, and transition metal cations)) and organic cations (e.g., ammonium cations, sulfonium cations, phosphonium cations, and pyridinium cations). An “anionic counterion” is a negatively charged atom or group associated with a cationic atom or group in order to maintain electronic neutrality. Exemplary anionic counterions include halide anions (e.g., F⁻, Cl⁻, Br⁻, and I⁻), NO₃⁻, ClO₄⁻OH⁻, H₂PO₄⁻, HSO4⁻, sulfonate anions (e.g., methansulfonate, trifluoromethane-sulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate anions (e.g., acetate, ethanoate, propanoate, benzoate, glycerate, lactate, tartrate, and glycolate). A counterion may be monovalent or multivalent (e.g., bivalent, trivalent, tetravalent, etc.).

The term “ionizable” refers to a compound containing at least one functional group that (a) bears a positive or negative charge (i.e., is “ionized”) and is therefore associated with a counterion of opposite charge, or (b) is electronically neutral but ionized at a higher or lower pH. Thus, ionizable compounds include quaternary ammonium salts as well as uncharged amines, and carboxylate moieties as well as uncharged carboxyl groups.

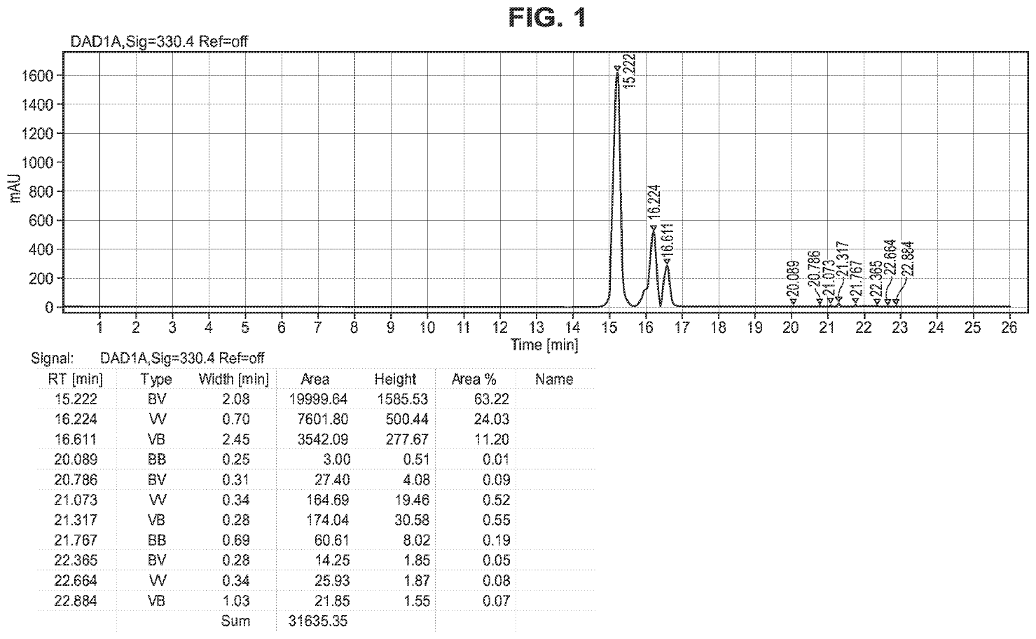

The term “carotenoid”, as used herein, refers to organic pigments which are structurally composed of a polyene hydrocarbon chain, and which may terminate in a ring. Carotenoids are divided into two classes, xanthophylls (which contain oxygen atoms) and carotenes (which contain no oxygen atoms). Non-limiting examples of carotenoids suitable for use in the provided compositions and methods are provided in FIG. 1A-FIG. 1D of WO2019213538A1, as applicable. Carotenoids with ionizable functional groups comprise naturally occurring carotenoid sulphates, carotenoid carboxylic acids/carboxylates, synthetic phosphates, blue carotenoid oxonium ions and blue carotenoproteins.

The term “Polyene Carotenoid” as used herein, refers to a carotenoid containing 3 or more conjugated double bonds, and methyl or low alkyl (C2-C3) substitutions. In some embodiments, “Polyene Carotenoid” can refer to a hydrocarbon group containing 3 or more conjugated double bonds and methyl substitutions and have a total number of 6-36 carbons, such as 8, 10, 12, 14, 16, 18, or 20 carbons.

The term “naturally occurring” refers to a compound or composition that occurs in nature, regardless of whether the compound or composition has been isolated from a natural source or chemically synthesized. Examples of naturally occurring carotenoid mono- and di-carboxylic acids include crocetin, norbixin, azafrin and neurosporaxanthin.

An “apocarotenoid” is a carotenoid degradation product in which the normal structure (e.g., C40) has been shortened by the removal of fragments from one or both ends. Examples of naturally occurring apocarotenoids include crocetin (C20), bixin (C25), Vitamin A, abscisic acid, mycorradicin and blumenin.

As used herein an “effective amount” refers to a dosage of an agent sufficient to provide a medically desirable result. The effective amount will vary with the desired outcome, the particular disease or condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose. In the case of cancer, the effective amount of an agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

The terms “hyperproliferative disorder”, “proliferative disease”, and “proliferative disorder”, are used interchangeably herein to pertain to an unwanted or uncontrolled cellular proliferation of excessive or abnormal cells which is undesired, such as, neoplastic or hyperplastic growth, whether in vitro or in vivo. In some embodiments, the proliferative disease is cancer or tumor disease (including benign or cancerous) and/or any metastases, wherever the cancer, tumor and/or the metastasis is located. In some embodiments, the proliferative disease is a benign or malignant tumor. In some embodiments, the proliferative disease is a non-cancerous disease. In some embodiments, the proliferative disease is a hyperproliferative condition such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

“Cancer,” “tumor,” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (metastasize) as well as any of a number of characteristic structural and/or molecular features. “Tumor,” as used herein refers to all neoplastic cell growth and proliferation, whether malignant or benign, and all pre-cancerous and cancerous cells and tissues. A “cancerous tumor,” or “malignant cell” is understood as a cell having specific structural properties, lacking differentiation and being capable of invasion and metastasis. A cancer that can be treated using a carotenoid pharmaceutical composition provided herein includes without limitation, a non-hematologic malignancy including such as for example, lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, and melanoma; and a hematologic malignancy such as for example, a leukemia, a lymphoma and other B cell malignancies, myeloma and other plasma cell dysplasias or dyscrasias. Other types of cancer and tumors that may be treated using a trans-crocetin composition are described herein or otherwise known in the art. The terms “cancer,” “cancerous,” “cell proliferative disorder,” “proliferative disorder,” and “tumor” are not mutually exclusive as referred to herein.

“Ischemia” relates to a restriction in blood supply to tissues or organs (tissue hypoperfusion) causing a shortage of oxygen needed for cellular metabolism. The term “ischemia injury”, as used herein, relates to the damage due to a shortage of oxygen needed for cellular metabolism.

“Reperfusion” refers to the restoration of blood flow to ischemic tissue.

The term “ischemia/reperfusion injury”, also known as “ischemia/reperfusion damage” relates to organ or tissue damage caused when blood supply returns to the organ or tissue after a period of ischemia. The absence of oxygen and nutrients from blood during the ischemic period creates a condition in which the restoration of circulation results in inflammation and oxidative damage through the induction of oxidative stress rather than restoration of normal function. Oxidative stress associated with reperfusion may cause damage to the affected tissues or organs. Ischemia/reperfusion injury is characterized biochemically by a depletion of oxygen during an ischemic event followed by reoxygenation and the concomitant generation of reactive oxygen species during reperfusion. Examples of ischemia injury or ischemia/reperfusion injury include organ dysfunction (in the ischemic organ or in any other organ), infarct, inflammation (in the damaged organ or tissue), oxidative damage, mitochondrial membrane potential damage, apoptosis, reperfusion-related arrhythmia, cardiac stunning, cardiac lipotoxicity, ischemia-derived scar formation, and combinations thereof. In some embodiments, ischemia/reperfusion injury is assessed by using oxidative stress biochemical markers such as malondialdehyde (MDA), high-sensitivity troponin T (hs-TnT), high-sensitivity troponin T (hs-Tnl), creatin kinase myocardial band (CK-MB), and the inflammatory cytokines TNF-alpha IL-1 beta, IL-6, and IL-10.

“Organ dysfunction” refers to a condition wherein a particular organ does not perform its expected function. An organ dysfunction develops into organ failure if the normal homeostasis cannot be maintained without external clinical intervention. Methods to determine organ dysfunction are known in the art and include without limitation, monitorization and scores including sequential organ failure assessment (SOFA) score, multiple organ dysfunction (MOD) score and logistic organ dysfunction (LOD) score.

Terms such as “treating,” or “treatment,” or “to treat” refer to both (a) therapeutic measures that cure, slow down, attenuate, lessen symptoms of, and/or halt progression of a diagnosed pathologic condition or disorder and (b) prophylactic or preventative measures that prevent and/or slow the development of a targeted disease or condition. Thus, subjects in need of treatment include those already with the cancer, disorder or disease; those at risk of having the cancer or condition; and those in whom the infection or condition is to be prevented. Subjects are identified as “having or at risk of having” sepsis, an infectious disease, a disorder of the immune system, a metabolic disorder (e.g., diabetes), a hyperproliferative disease, or another disease or disorder referred to herein using well-known medical and diagnostic techniques. In certain embodiments, a subject is successfully “treated” according to the methods provided herein if the subject shows, e.g., total, partial, or transient amelioration or elimination of a symptom associated with the disease or condition (e.g., cancer and arthritis such as rheumatoid arthritis). In specific embodiments, the terms “treating,” or “treatment,” or “to treat” refer to the amelioration of at least one measurable physical parameter of a proliferative disorder, such as growth of a tumor, not necessarily discernible by the patient. In other embodiments, the terms “treating,” or “treatment,” or “to treat” refer to the inhibition of the progression of a proliferative disorder, either physically by, e.g., stabilization of a discernible symptom, physiologically by, e.g., stabilization of a physical parameter, or both. In other embodiments, the terms “treating,” or “treatment,” or “to treat” refer to the reduction or stabilization of tumor size, tumor cell proliferation or survival, or cancerous cell count. Treatment can be with a provided pharmaceutical composition disclosed herein (e.g., a liposomal trans-crocetinate) alone, or in combination with an additional therapeutic agent.

“Subject” and “patient,” and “animal” are used interchangeably and refer to mammals such as human patients and non-human primates, as well as experimental animals such as rabbits, rats, and mice, and other animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

The term “elderly” refers to an aged subject, who has passed middle age. In one embodiment, an elderly mammalian subject is a subject that has survived more than two-thirds of the normal lifespan for that mammalian species. In a further embodiment, for humans, an aged or elderly subject is more than 65 years of age, such as a subject of more than 70, more than 75, more than 80 years of age. In yet another embodiment, for mice, an elderly mouse is from about 14 to about 18 months of age.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent”: the therapeutic agent or therapeutic agents used according to the disclosed compositions and methods can include any agent directed to treat a condition in a subject. Examples of therapeutic agents that may be suitable for use in accordance with the disclosed methods include vitamin C, thiamine, hydrocortisone or another corticosteroid (e.g., a glucocorticoid such as, cortisone, ethamethasoneb, prednisone, prednisolone, triamcinolone, dexamethasone and methylprednisolone; and mineralocorticoids such as fludrocortisonel), astaxanthin, abscisic acid, vitamin A, angiotensin II (e.g., GIAPREZA™), tissue plasminogen activator (tPA), an antimicrobial (e.g., antibiotic or chloroquin and its analogs) and an anti-inflammatory.

Additional examples of therapeutic agents that may be suitable for use in accordance with the disclosed methods include, without limitation, anti-restenosis, pro- or anti-proliferative, anti-neoplastic, antimitotic, anti-platelet, anticoagulant, antifibrin, antithrombin, cytostatic, antibiotic and other anti-infective agents, anti-enzymatic, anti-metabolic, angiogenic, cytoprotective, angiotensin converting enzyme (ACE) inhibiting, angiotensin II receptor antagonizing and/or cardioprotective agents. In general, any therapeutic agent known in the art can be used, including without limitation agents listed in the United States Pharmacopeia (U.S.P.), Goodman and Gilman's The Pharmacological Basis of Therapeutics, 10th Ed., McGraw Hill, 2001; Katzung, Ed., Basic and Clinical Pharmacology, McGraw-Hill/Appleton & Lange, 8th ed., Sep. 21, 2000; Physician's Desk Reference (Thomson Publishing; and/or The Merck Manual of Diagnosis and Therapy, 18th ed., 2006, Beers and Berkow, Eds., Merck Publishing Group; or, in the case of animals, The Merck Veterinary Manual, 9th ed., Kahn Ed., Merck Publishing Group, 2005; all of which are incorporated herein by reference used herein to refer to an agent or a derivative thereof that can interact with a hyperproliferative cell such as a cancer cell or an immune cell, thereby reducing the proliferative status of the cell and/or killing the cell. Examples of therapeutic agents include, but are not limited to, chemotherapeutic agents, cytotoxic agents, platinum-based agents (e.g., cisplatin, carboplatin, oxaliplatin), taxanes (e.g., Taxol), etoposide, alkylating agents (e.g., cyclophosphamide, ifosamide), metabolic antagonists (e.g., methotrexate (MTX), 5-fluorouracil, gemcitabine, pemetrexed, or derivatives thereof), antitumor antibiotics (e.g., mitomycin, doxorubicin), plant-derived antitumor agents (e.g., vincristine, vindesine, Taxol). Such agents may further include, but are not limited to, the anticancer agents trimetrexate, TEMOZOLOMIDE™, RALTRITREXED™, S-(4-Nitrobenzyl)-6-thioinosine (NBMPR), 6-benzyguanidine (6-BG), bis-chloronitrosourea (BCNU) and CAMPTOTHECIN™, or a therapeutic derivative of any thereof. “Therapeutic agents” also refer to salts, acids, and free based forms of the above agents.

The term “kit” refers to a set of two or more components necessary for employing the methods and compositions provided herein. Kit components can include, but are not limited to, carotenoids, polyethylene glycol, and aqueous solutions, liposome compositions and pharmaceutical compositions disclosed herein, reagents, buffers, containers and/or equipment.

The term “radiosensitizing agent” means a compound that makes tumor cells more sensitive to radiation therapy. Examples of radiosensitizing agents include misonidazole, metronidazole, tirapazamine, and trans-crocetin.

Methods of Preparing Carotenoid

Some embodiments of the present disclosure are directed to methods of preparing carotenoids. Typically, the carotenoids prepared by the methods described herein contain at least one carboxylic acid or ester functional group. The carotenoids can be generally prepared by reacting a suitable aldehyde with an olefin forming agent. The preparation methods described herein are advantageous in many aspects, for example, 1) a high overall yield, 2) an easy purification procedure, 3) can be readily adapted for large-scale manufacturing process, and 4) the final product is in high purity.

In some embodiments, the disclosure provides a method of preparing a carotenoid having Formula I:

Q-Y-Polyene Carotenoid-Y-Q, (I)

wherein,

the Polyene Carotenoid comprises
(a) 3, 4, 5, 6, 7, 8, 9, 10, 3-5, 6-8, 9-10, or more than 9, conjugated double bonds, and
(b) methyl or low alkyl (C2-C3) substitutions.
each Y is independently a bond,

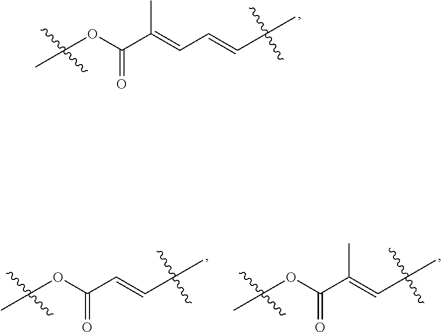

or a geometric isomer thereof, provided that at least one Y has a —C(O)—O— functional group, and
each Q is independently an ionizable group, hydrogen, an optionally substituted C₁₋₄ alkyl, or a counterion. To be clear, when Y contains a —C(O)—O— functional group, the O is linked with Q, i.e., —C(O)—O-Q. In some embodiments, one Y group has a —C(O)—O— functional group. In some embodiments, both Y groups include a —C(O)—O— functional group. In some embodiments, both Y are independently selected from

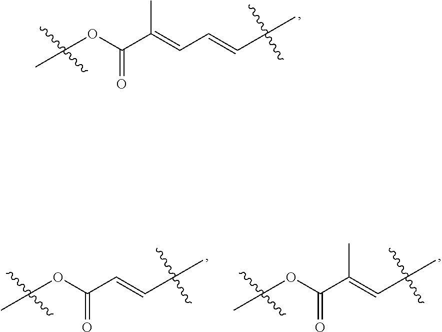

or a geometric isomer thereof. In some embodiments, both Y are the same. In some embodiments, both Q are the same. In some embodiments, both Q are C₁₋₄ alkyl such as methyl, ethyl, isopropy, tert-butyl, etc. In some embodiments, both Q are hydrogen. In some embodiments, both Q are monovalent counterion, such as alkali metal ion, such as Li⁺, Na⁺, or K⁺, NH₄⁺, or a protonated organic amine. In some embodiments, when Y is a bond, the Q is directly connected with the Polyene Carotenoid, and Q can be an ionizable group as defined herein.

In some embodiments, the carotenoid having Formula I can be characeterized as having a formula according to Formula I-A to I-F, or a geometric isomer thereof:

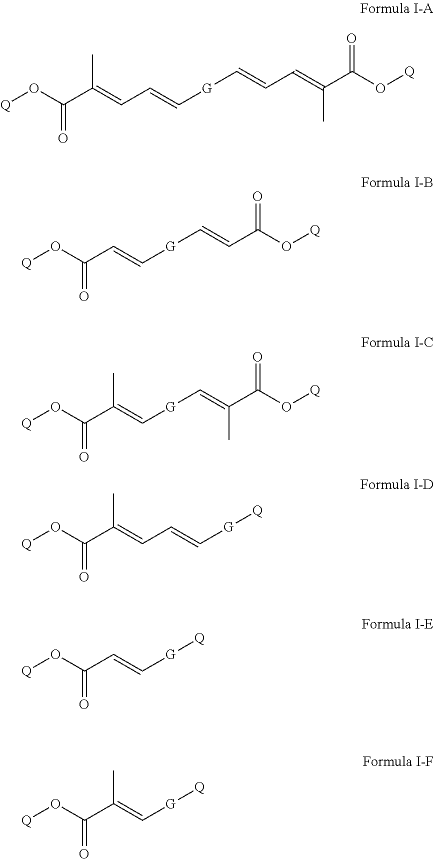

wherein G is the Polyene Carotenoid as defined herein, and Q is defined herein (e.g., as shown in [4]-[8] in the Brief Summary). In some embodiments, the Polyene Carotenoid is a hydrocarbon group having a total number of 6-36 carbons, such as 8, 10, 12, 14, 16, 18, or 20 carbons.

In some embodiments, the carotenoid of Formula I can be characterized as having a formula according to Formula I-A, I-B, or I-C as defined herein. In Formula I-A, I-B, or I-C, both Q are typically the same. For example, in some embodiments, both Q are C₁₋₄ alkyl such as methyl, ethyl, isopropy, tert-butyl, etc. In some embodiments, both Q can be hydrogen. In some embodiments, both Q can also be monovalent counterion, such as alkali metal ion, such as Li⁺, Na⁺, or K⁺, NH₄⁺, or a protonated organic amine. In Formula I-A, I-B, or I-C, the Polyene Carotenoid is typically a hydrocarbon group having a total number of 6-36 carbons, such as 8, 10, 12, 14, 16, 18, or 20 carbons.

Typically, the method herein for preparing the compound of Formula I (e.g., Formula I-A to I-F) comprises:
a. reacting an aldehyde of Formula II with an olefin forming agent to form an ester (e.g., a C₁₋₄ alkyl ester, such as ethyl ester):

Q²-Y²-Polyene Carotenoid-Y²-Q², (II)
wherein at least one of Q²-Y² is HC(O)—, i.e., one or both Q²-Y² are HC(O)—, and when only one of Q²-Y² is HC(O)—, the other Q²-Y² is the same as Y-Q- in Formula I,
wherein the olefin forming agent is capable of reacting with the aldehyde to form a structural moiety of

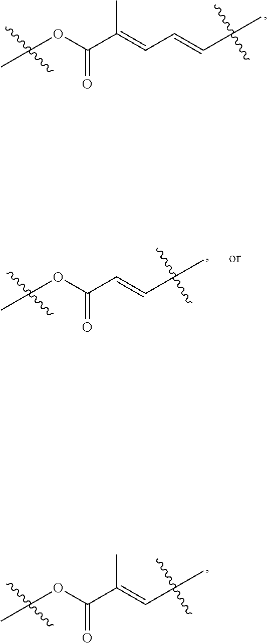

or a geometric isomer thereof,
and
b. optionally, when at least one Q is hydrogen or a counterion, hydrolyzing the ester to form the carotenoid of Formula I. In some embodiments, Q is a counterion and the carotenoid of Formula I is a salt (e.g., described herein, such as a monovalent or divalent salt). In such embodiments, the hydrolyzing can be carried out such that the salt can be isolated and purified without first converting into the free acid form (i.e., COOQ is COOH). However, in some embodiments, the salt can also be prepared from the free acid form by reacting with an appropriate base.

In the methods described herein, the reaction between the aldehyde of Formula II and the olefin forming agent forms an ester having a structural moiety of

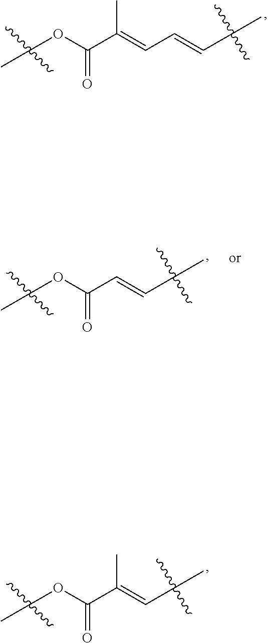

or a geometric isomer thereof. The isomeric purity of the newly formed olefin bond (C═C) in this reaction step typically can have an impact on the isomeric purity of the carotenoid product and the easiness of purification of the carotenoid product. In some embodiments, the ester formed from the aldehyde and the olefin forming agent can be selected from:

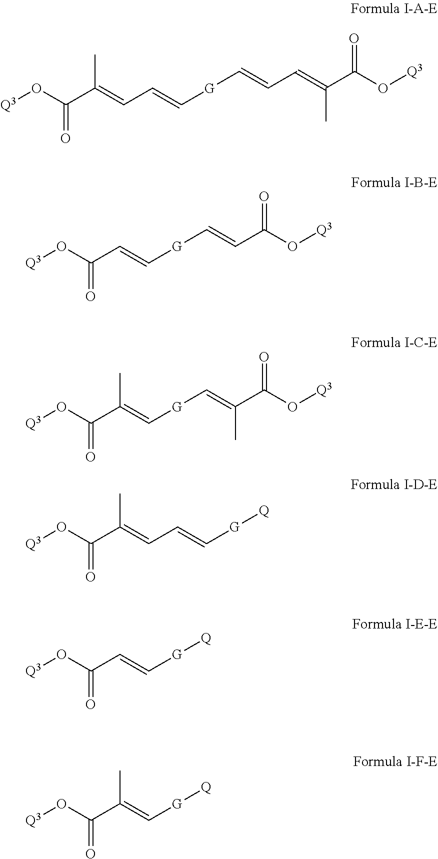

wherein G is the Polyene Carotenoid as defined herein, Q³ is an optionally substituted C₁₋₄ alkyl (e.g., methyl, ethyl, isopropyl, tert-butyl, etc.) and Q is defined herein. The esters of Formula I-A-E to I-F-E in the table can be converted into the carotenoid having Formula I-A to I-F, wherein at least one Q is hydrogen or a counterion, respectively, through hydrolysis of the ester bond.

Typically, the olefin forming agent is a phosphine based olefin forming agent (e.g., as shown in [11]-[18] of the Brief Summary), which can react with the aldehyde of Formula II to form a double bond (C═C) through a Wittig type reaction.

The olefin forming agent can be selected based on the desired target molecule. For example, when the carotenoid contains a moiety of

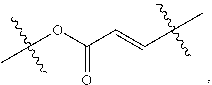 [SMILES: */C=C/C(=O)O*]

those skilled in the art would understand that a reaction of the aldehyde of Formula II with an olefin forming agent, such as those of Formula III-1 (as defined herein) and the alike would be appropriate

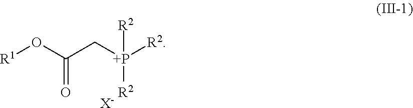

Typically, in Formula III-1, R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide. In some embodiments, R¹ is a C₁₋₄ alkyl, such as methyl, ethyl, isopropyl, tert-butyl, etc. In some embodiments, R¹ is not methyl. In some embodiments, R¹ is ethyl. The R² group for Formula III-1 is not particularly limited. Typically, the three R² groups in Formula III-1 are the same, although different R² groups can also be used. In some embodiments, each of the R² groups is phenyl, and the compound of Formula III-1 can have a Formula III-1-A:

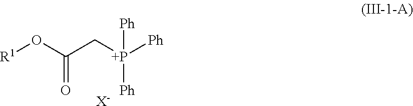

wherein R¹ is an optionally substituted C₁₋₄ alkyl (e.g., ethyl), and X⁻ is a counterion, e.g., a halide such as chloride or bromide. In some embodiments, the isolated deprotonated ylide can also be used, which may be represented by Formula III-1-Y:

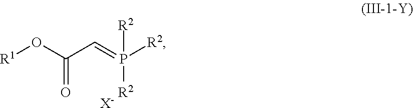

wherein R¹ and R² are defined hereinabove. However, in some embodiments, the compound of Formula III-1 can be directly used, typically in combination with a base such as an inorganic base, such as a carbonate base, such as potassium carbonate, sodium carbonate, potassium bicarbonate, sodium bicarbonate, etc. Other suitable base include any of those known in the art suitable for similar bond formations.

Similarly, when the carotenoid contains a moiety of

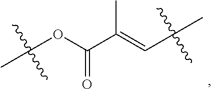 [SMILES: */C=C(\C)C(=O)O*]

those skilled in the art would understand that a reaction of the aldehyde of Formula II with an olefin forming agent, such as those of Formula III-2 (as defined herein) and the alike would be appropriate:

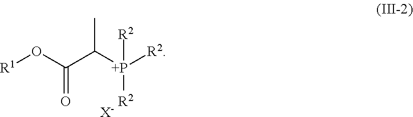

Typically, in Formula III-2, R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide. In some embodiments, R¹ is a C₁₋₄ alkyl, such as methyl, ethyl, isopropyl, tert-butyl, etc. In some embodiments, R¹ is not methyl. In some embodiments, R¹ is ethyl. The R² group for Formula III-2 is not particularly limited. Typically, the three R² groups in Formula III-2 are the same, although different R² groups can also be used. In some embodiments, each of the R² groups is phenyl, and the compound of Formula III-2 can have a Formula III-2-A:

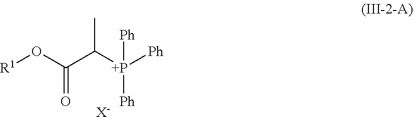

wherein R¹ is an optionally substituted C₁₋₄ alkyl (e.g., ethyl), and X⁻ is a counterion, e.g., a halide such as chloride or bromide. In some embodiments, the isolated deprotonated ylide can also be used, which may be represented by

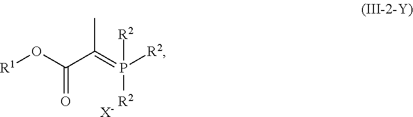

wherein R¹ and R² are defined hereinabove. However, in some embodiments, the compound of Formula III-2 can be directly used, typically in combination with a base such as an inorganic base, such as a carbonate base, such as potassium carbonate, sodium carbonate, potassium bicarbonate, sodium bicarbonate, etc. Other suitable base include any of those known in the art suitable for similar bond formations.

When the carotenoid contains a moiety of

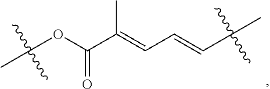 [SMILES: */C=C/C=C(\C)C(=O)O*]

those skilled in the art would understand that a reaction of the aldehyde of Formula II with an olefin forming agent, such as those of Formula III-3 (as defined herein) and the alike would be appropriate:

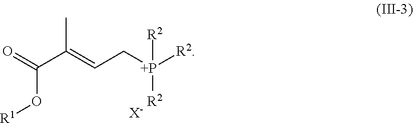

Typically, in Formula III-3, R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide. In some embodiments, R¹ is a C₁₋₄ alkyl, such as methyl, ethyl, isopropyl, tert-butyl, etc. In some embodiments, R¹ is not methyl. In some embodiments, R¹ is ethyl. The R² group for Formula III-3 is not particularly limited. Typically, the three R² groups in Formula III-3 are the same, although different R² groups can also be used. In some embodiments, each of the R² groups is phenyl, and the compound of Formula III-3 can have a Formula III-3-A:

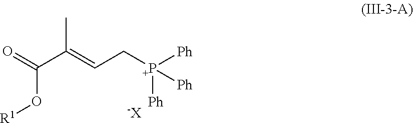

wherein R¹ is an optionally substituted C₁₋₄ alkyl (e.g., ethyl), and X⁻ is a counterion, e.g., a halide such as chloride or bromide. In some embodiments, the isolated deprotonated ylide can also be used, which may be represented by Formula III-3-Y:

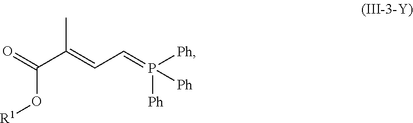

wherein R¹ and R² are defined hereinabove. However, in some embodiments, the compound of Formula III-3 can be directly used, typically in combination with a base such as an inorganic base, such as a carbonate base, such as potassium carbonate, sodium carbonate, potassium bicarbonate, sodium bicarbonate, etc. Other suitable base include any of those known in the art suitable for similar bond formations.

The phosphorus based olefin forming agents are typically available through commercial source or can be prepared by known procedures or adapted from such procedures. In general, a phosphine (such as triphenyl phosphine) can react with a halide, such as

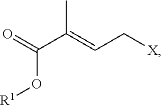

wherein X is chloro or bromo, to form an olefin forming agent, such as Formula III-3.

The reaction between the olefin forming agent and the aldehyde of Formula II can be typically carried out in an organic solvent under heat in the presence of a base such as an inorganic base. However, it was unexpected found that the solvent system used in the olefin forming process can have a significant impact on the efficiency of this process. As shown in the Examples section, in one example, the use of acetonitrile and toluene as solvent system greatly improved the efficiency of separating the desired isomer. As shown in the examples, the desired isomer can be separated from the reaction mixture relatively easily, with simple processes such as filtration, washing, slurry, etc. And the diester produced in the example showed greater than 98% purity by HPLC area percentage at either detection wavelength, 254 nm or 420 nm. In contrast, in U.S. Pat. No. 8,030,350, a similar reaction carried in butylene oxide and toluene, the reaction provided a cis/trans ratio of about 60:40 and the product purified contained about 5% to 9% cis isomers in scale up. Because of this improvement, the process which uses acetonitrile and toluene as solvent can be easily scaled up to meet the demand from commercial manufacturing.

According, in some embodiments, the olefin forming reaction (i.e., the reaction between the olefin forming agent (e.g., any of Formula III-1 to III-3) and the aldehyde of Formula II) can be carried out in a non-ether solvent in the presence of a base such as an inorganic base. In some embodiments, the olefin forming reaction can be carried out in a solvent system comprising acetonitrile. In some specific embodiments, the olefin forming reaction can be carried out in a solvent system comprising acetonitrile and toluene. Typically, the acetonitrile and toluene can be used in a ratio of about 1:20 to 20:1, such as about 1:20, about 1:15, about 1:10, about 1:5, about 1:3, about 1:2, about 1:1, about 2:1, about 5:1, about 10:1, about 15:1, or any ranges between the recited values, such as about 1:5-1:2, about 1:10-1:1, etc.

The olefin forming reaction is typically carried out under heat, e.g., with a reaction temperature of about ranging from about 50° C.-100° C. In some embodiments, the olefin forming reaction can be carried out in two steps, the first step will include the nucleophilic addition of the aldehyde with the olefin forming agent to form an intermediate, which is then followed by elimination to form a double bond, for example, triphenylphosphine oxide is eliminated when the olefin forming agent is a compound of Formula III-3-A. Thus, in some embodiments, the olefin forming reaction can be carried out initially at a lower temperature to allow the first step to take place, for example, the reaction temperature can be about 45° C. to about 65° C., such as about 60° C., for a first period of time ranging from about 1 hour to about 6 hours, such as about 2, 3, 4, or 5 hours; and subsequently, the reaction temperature can be raised to about 70° C. to about 100° C., such as about 80° C., for a second period of time of ranging from about 10 hours to about 48 hours, such as about 12, 14, 16, 18, 20, or 24 hours. Typically, the time needed for each step can be monitored, for example, by monitoring the disappearance of starting material or intermediate.

The olefin forming reaction herein is typically carried out in the presence of a base such as an inorganic base. For example, in some embodiments, the olefin forming reaction is carried out in the presence of a carbonate base, e.g., sodium carbonate, potassium carbonate, sodium bicarbonate, potassium bicarbonate, etc. The amount of base is typically in excess of the stoichiometric amount, for example, about 30-50% in excess or 100% in excess or more.

As discussed above, the product from the olefin forming reaction is typically an ester, for example, those having Formula I-A-E, I-B-E, I-C-E, I-D-E, I-E-E, I-F-E, as defined herein. The ester can be typically isolated from the olefin forming reaction in a substantially pure form. For example, in some embodiments, the ester can be isolated in a purity of greater than 95%, preferably, greater than 97%, greater than 98%, greater than 99%, by HPLC area percentage with detection wavelength at 254 nm and 420 nm. For the avoidance of doubt, when it is said that a carotenoid herein has a certain purity profile by HPLC area percentage with detection wavelength at 254 nm and 420 nm, it should be understood as such that the purity profile is satisfied only if the purity profile is observed when the detection wavelength is 254 nm and when the detection wavelength is 420 nm. For example, a purity of greater than 99% by HPLC area percentage with detection wavelength at 254 nm and 420 nm is only satisfied when the purity is determined to be greater than 99% by HPLC area percentage when using 254 nm as detection wavelength and when using 420 nm as detection wavelength. Other similar expressions should be understood similarly.

In some embodiments, either one or both Q in Formula I is hydrogen or a counterion, such as a monovalent counterion (e.g., Li⁺, Na⁺, or K⁺) or a divalent counterion (e.g., Mg²⁺, Ca²⁺, etc.). In such embodiments, the method herein further comprises hydrolyzing the ester from the olefin forming reaction to provide the carotenoid of Formula I. The hydrolyzing is typically carried out in an alcoholic solvent under heat in the presence of an alkali hydroxide base, such as LiOH, NaOH, or KOH, typically in aqueous solution. In cases where Q is Li⁺, Na⁺, or K⁺, the carotenoid salt can be isolated directly from the hydrolysis reaction using LiOH, NaOH, or KOH, respectively, without converting it into the free acid first. In some embodiments, the carotenoid salt can also be prepared from the free acid form by reacting with an appropriate base. Although base is typically used for the hydrolysis for the methods herein, in some embodiments, an acid hydrolysis is also feasible, for example, when the ester formed is a tert-butyl ester.

In some embodiments, the method herein is for preparing a carotenoid of Formula I-1:

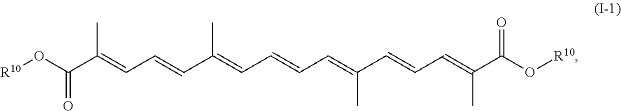

or a geometric isomer thereof, or a combination thereof,
wherein each R¹⁰ is independently selected from hydrogen, a counterion (e.g., a monovalent counterion or a divalent counterion), and an optionally substituted C₁₋₄ alkyl. In some embodiments, both R¹⁰ are the same. In some embodiments, both R¹⁰ are C₁₋₄ alkyl, preferably, ethyl. In some embodiments, both R¹⁰ are a monovalent counterion, preferably, an alkali metal ion, such as Li⁺, Na⁺, or K⁺. In some embodiments, both R¹⁰ are hydrogen. Typically, the method comprises reacting a dialdehyde of Formula II-1:

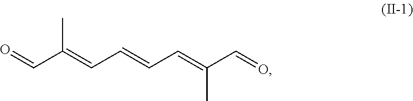

or a geometric isomer thereof, or a combination thereof, with an olefin forming agent described herein (e.g., Formula III-1 or III-1-A), for example, under any of the olefin forming reaction conditions described herein. Typically, the dialdehyde is in a substantially pure form of Formula II-1:

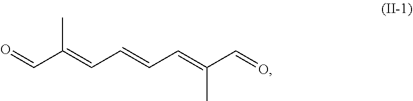

which includes less than 10% of other geometric isomers, by HPLC area percentage with detection wavelength at 330 nm, preferably, less than 5% such as less than 3%, or less than 2% of other geometric isomers. In some embodiments, either one or both R¹⁰ in Formula I-1 is hydrogen or a counterion, such as a monovalent counterion (e.g., Li⁺, Na⁺, or K⁺) or a divalent counterion (e.g., Mg²⁺, Ca²⁺, etc.). In such embodiments, the method herein further comprises hydrolyzing the ester from the olefin forming reaction to provide the carotenoid of Formula I-1. The hydrolyzing is typically carried out in an alcoholic solvent under heat in the presence of an alkali hydroxide base, such as LiOH, NaOH, or KOH, typically in aqueous solution. In cases where Q is Li⁺, Na⁺, or K⁺, the carotenoid salt can be isolated directly from the hydrolysis reaction using LiOH, NaOH, or KOH, respectively, without converting it into the free acid first. In some embodiments, the carotenoid salt can also be prepared from the free acid form by reacting with an appropriate base. Although base is typically used for the hydrolysis for the methods herein, in some embodiments, an acid hydrolysis is also feasible, for example, when the ester formed is a tert-butyl ester.

In some embodiments, the method herein is for preparing a carotenoid of Formula I-2:

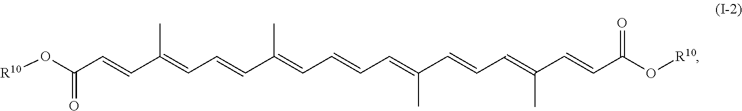

or a geometric isomer thereof, or a combination thereof,
wherein each R¹⁰ is independently selected from hydrogen, a counterion (e.g., a monovalent counterion or a divalent counterion), and an optionally substituted C₁₋₄ alkyl. In some embodiments, both R¹⁰ are the same. In some embodiments, both R¹⁰ are C₁₋₄ alkyl, preferably, ethyl. In some embodiments, both R¹⁰ are a monovalent counterion, preferably, an alkali metal ion, such as Li⁺, Na⁺, or K⁺. In some embodiments, both R¹⁰ are hydrogen. Typically, the method comprises reacting a dialdehyde of Formula II-2:

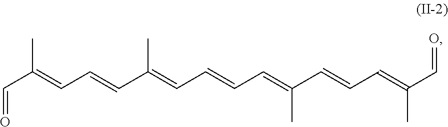 [SMILES: C\C(C=O)=C/C=C/C(C)=C/C=C/C=C(C)/C=C/C=C(\C)C=O]

or a geometric isomer thereof, or a combination thereof, with an olefin forming agent described herein (e.g., Formula III-1 or III-1-A), for example, under any of the olefin forming reaction conditions described herein. Typically, the dialdehyde is in a substantially pure form of Formula II-2:

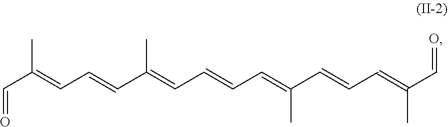 [SMILES: C\C(C=O)=C/C=C/C(C)=C/C=C/C=C(C)/C=C/C=C(\C)C=O]

which includes less than 10% of other geometric isomers, by HPLC area percentage with detection wavelength at 254 nm and 420 nm, preferably, less than 5% such as less than 3%, or less than 2% of other geometric isomers. In some embodiments, either one or both R¹⁰ in Formula I-2 is hydrogen or a counterion, such as a monovalent counterion (e.g., Li⁺, Na⁺, or K⁺) or a divalent counterion (e.g., Mg²⁺, Ca²⁺, etc.). In such embodiments, the method herein further comprises hydrolyzing the ester from the olefin forming reaction to provide the carotenoid of Formula I-2. The hydrolyzing is typically carried out in an alcoholic solvent under heat in the presence of an alkali hydroxide base, such as LiOH, NaOH, or KOH, typically in aqueous solution. In cases where Q is Li⁺, Na⁺, or K⁺, the carotenoid salt can be isolated directly from the hydrolysis reaction using LiOH, NaOH, or KOH, respectively, without converting it into the free acid first. In some embodiments, the carotenoid salt can also be prepared from the free acid form by reacting with an appropriate base. Although base is typically used for the hydrolysis for the methods herein, in some embodiments, an acid hydrolysis is also feasible, for example, when the ester formed is a tert-butyl ester.

The compound of Formula I produced by the methods described herein can be typically isolated in a substantially pure form. For example, in some embodiments, the compound of Formula I (e.g., Formula I-1 or I-2) can be isolated in a purity of greater than 95%, preferably, greater than 97%, greater than 98%, greater than 99%, by HPLC area percentage with detection wavelength at 254 nm and 420 nm.

Methods of Preparing Crocetin

Some more specific embodiments of the disclosure are directed to methods of preparing crocetin, including its esters and salts, which can be represented by Formula I-1:

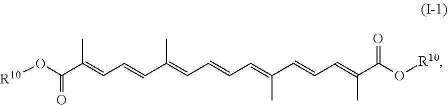

wherein each R¹⁰ is independently selected from hydrogen, a counterion (e.g., a monovalent counterion or a divalent counterion), and an optionally substituted C₁₋₄ alkyl. In some embodiments, both R¹⁰ are the same. In some embodiments, one or both R¹⁰ are C₁₋₄ alkyl (e.g., methyl, ethyl, isopropyl, tert-butyl, etc.). In some embodiments, one or both R¹⁰ are ethyl. In some embodiments, one or both R¹⁰ are hydrogen. In some embodiments, one or both R¹⁰ are a monovalent counterion, preferably, an alkali metal ion, such as Li⁺, Na⁺, or K⁺.

The methods of preparing crocetin herein is typically characterized in that the crocetin prepared is in a substantially pure form. For example, in some embodiments, the crocetin prepared can be characterized as having less than 3% of other geometric isomers (total amount, other than the crocetin as drawn in Formula I-1), by HPLC area percentage with detection wavelength at 254 nm and 420 nm, preferably, less than 2% such as less than 1.5%, or less than 1% of other geometric isomers. In some embodiments, the other geometric isomers are not detectable by the analytical methods described herein. In some embodiments, the other geometric isomers are in an amount of about 0.01%, about 0.05%, about 0.1%, about 0.2%, about 0.3%, about 0.4%, about 0.5%, about 0.6%, about 0.7%, about 0.8%, about 0.9%, about 1%, or any ranges between the recited values, such as about 0.1-1%, about 0.01-0.8%, about 0.1-0.8%, about 0.3-0.8%, etc. by HPLC area percentage with detection wavelength at 254 nm and 420 nm. In some embodiments, the crocetin prepared can also be characterized as having a purity of greater than 95%, preferably, greater than 97%, greater than 98%, greater than 99%, by HPLC area percentage with detection wavelength at 254 nm and 420 nm. In some embodiments, the purity can be up to about 99.5% or up to about 99.9%. In some embodiments, the crocetin prepared can also be characterized as having a purity of about 95%, about 97%, about 98%, about 98.5%, about 99%, about 99.5%, about 99.9%, or any range between the recited value, such as about 98.5-99.5% or about 99-99.9%, by HPLC area percentage with detection wavelength at 254 nm and 420 nm. U.S. Pat. No. 8,030,350 describes that its HPLC purity of the trans sodium crocetinate (TSC) is only about 97.56% or about 94.21%, without specifying how much other geometric isomers are present in its composition. It is also noted that the ethyl ester of crocetin described in U.S. Pat. No. 8,030,350 contains about 5-9% of other geometric isomers in its scale up process. Thus, at the very least, the methods herein show advantages in providing crocetin esters and salts with a higher purity—following the synthetic process described in U.S. Pat. No. 8,030,350 would not lead to this level of purity.

In some embodiments, the present disclosure provides a method of preparing crocetin having formula I-1:

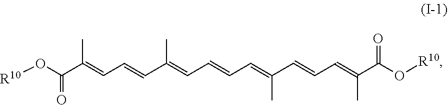

which is in a substantially pure form (e.g., described herein), such as having less than 3% of other geometric isomers, by HPLC area percentage with detection wavelength at 254 nm and 420 nm, preferably, less than 2% such as less than 1.5%, or less than 1% of other geometric isomers, the method comprising:
a. reacting a dialdehyde of Formula II-1:

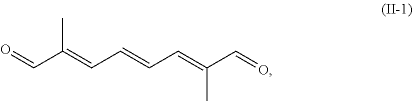

wherein the dialdehyde contains less than 10% of other geometric isomers, by HPLC area percentage with detection wavelength at 330 nm, preferably, less than 5% such as less than 3%, or less than 2% of other geometric isomers,
with an olefin forming agent of Formula III-3,

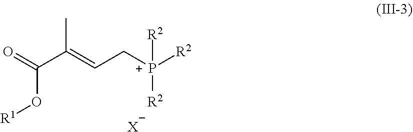

wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
to form an intermediate compound of Formula IV-1,

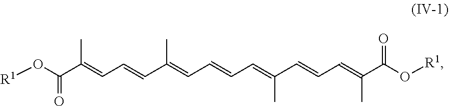

b. isolating the intermediate compound of Formula IV-1 in a substantially pure form (e.g., described herein), preferably, characterized as having less than 3% of other geometric isomers, by HPLC area percentage with detection wavelength at 254 nm and 420 nm, preferably, less than 2% such as less than 1.5%, or less than 1% of other geometric isomers, and
c. optionally hydrolyzing the intermediate compound of Formula IV-1 into the compound of Formula I-1 when at least one R¹⁰ is hydrogen or a counterion.

Typically, in Formula III-3, R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide. In some embodiments, R¹ is a C₁₋₄ alkyl, such as methyl, ethyl, isopropyl, tert-butyl, etc. In some embodiments, R¹ is not methyl. In some embodiments, R¹ is ethyl. The R² group for Formula III-3 is not particularly limited. Typically, the three R² groups in Formula III-3 are the same, although different R² groups can also be used. In some embodiments, each of the R² groups is phenyl, and the compound of Formula III-3 can have a Formula III-3-A:

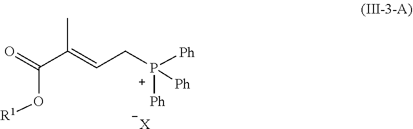

wherein R¹ is an optionally substituted C₁₋₄ alkyl, and X⁻ is a counterion, e.g., a halide such as chloride or bromide. In some embodiments, the isolated deprotonated ylide can also be used, which may be represented by Formula III-3-Y:

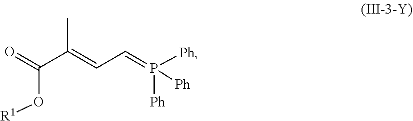

wherein R¹ is defined hereinabove. However, in some embodiments, the compound of Formula III-3 can be directly used, typically in combination with a base such as an inorganic base, such as a carbonate base, such as potassium carbonate, sodium carbonate, potassium bicarbonate, sodium bicarbonate, etc. Other suitable base include any of those known in the art suitable for similar bond formations.

The olefin forming agents of Formula III-3 are typically available through commercial source or can be prepared by known procedures or adapted from such procedures. In general, a phosphine (such as triphenyl phosphine) can react with a halide, such as

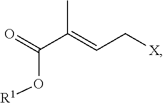

wherein X is chloro or bromo, to form an olefin forming agent of Formula III-3.

Typically, the dialdehyde of Formula II-1 used for the olefin forming reaction with Formula III-1 is in a substantially pure form. In some embodiments, the dialdehyde of Formula II-1 preferably includes less than 5% such as less than 3%, or less than 2% of other geometric isomers (total amount, other than the isomer as drawn in Formula II-1), by HPLC area percentage with detection wavelength at 330 nm. In some embodiments, the dialdehyde of Formula II-1 can also be characterized as having a purity of greater than 90%, such as greater than 91%, greater than 92%, greater than 93%, greater than 94%, greater than 95%, greater than 96%, greater than 97%, greater than 98%, and up to 99% or 99.5%, by HPLC area percentage with detection wavelength at 330 nm. For example, the dialdehyde of Formula II-1 can be characterized as having a purity of about 90%, about 91%, about 92%, about 93%, about 94%, about 95%, about 96%, about 97%, about 98%, about 99%, about 99.5%, or any ranges between the recited values such as about 90-98%, about 95-97%, etc., by HPLC area percentage with detection wavelength at 330 nm. In some embodiments, the HPLC purities of the dialdehyde of Formula II-1 can be determined by following the HPLC method described herein in the Examples section (Step 1 or 2).

The dialdehyde of Formula II-1 with the recited purity herein can be typically prepared from converting the geometric isomers of Formula II-1 into Formula II-1. For example, in some embodiments, the dialdehyde can be prepared by a process comprising isomerizing one or more geometric isomers of Formula II-1, e.g., under an acidic condition, such as with a catalytic amount of phenylsulfinic acid (e.g., generation from sodium phenylsulfinate and HCl), or other acids with similar pKa. An exemplary procedure is shown in the Examples section. In a typical process, a mixture of geometric isomers (Formula II-1-Mix) can be prepared from the acetal precursor (Formula II-1-Ace) under acidic conditions such as with H₂SO₄:

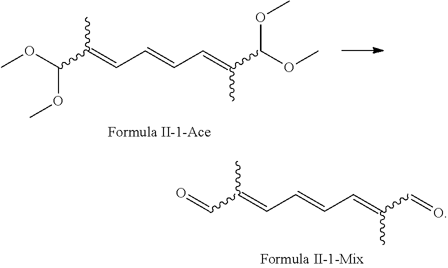

This mixture of geometric isomers can be isomerized by treatment with a catalytic amount of acid, typically also under heat to reach a thermodynamic equilibrium favoring the isomer as drawn in Formula II-1. For example, in some embodiments, the isomerizing can use a catalytic amount of acid formed from sodium phenyl sulfinate and hydrochloric acid. In some embodiments, the sodium phenyl sulfinate can also be in a slightly excess of the hydrochloric acid, which thus leaves both phenyl sulfinic acid and phenyl sulfinate in the reaction mixture. The isomerization reaction is not limited to a particular solvent. In some embodiments, the solvent can be 1,4-dioxane. The reaction temperature can typically range from about 50° C. to about 100° C., such as about 75° C. The isomerization reaction is typically carried out at a duration such that the desired isomer (Formula II-1) is no longer increased. The dialdehyde can be further purified by recrystallization, e.g., from an ether solvent such as tetrahydrofuran.

The reaction between the olefin forming agent of Formula III-1 and the dialdehyde of Formula II-1 can be typically carried out in an organic solvent under heat in the presence of a base such as a base such as an inorganic base.

In some embodiments, the olefin forming reaction between the olefin forming agent of Formula III-1 and the dialdehyde of Formula II-1 can be carried out in a non-ether solvent in the presence of a base such as an inorganic base. In some embodiments, the olefin forming reaction between the olefin forming agent of Formula III-1 and the dialdehyde of Formula II-1 can be carried out in a solvent system comprising acetonitrile. In some specific embodiments, the olefin forming reaction between the olefin forming agent of Formula III-1 and the dialdehyde of Formula II-1 can be carried out in a solvent system comprising acetonitrile and toluene. Typically, the acetonitrile and toluene can be used in a ratio of about 1:20 to 20:1, such as about 1:20, about 1:15, about 1:10, about 1:5, about 1:3, about 1:2, about 1:1, about 2:1, about 5:1, about 10:1, about 15:1, or any ranges between the recited values, such as about 1:5-1:2, 1:10-1:1, etc.

The olefin forming reaction between the olefin forming agent of Formula III-1 and the dialdehyde of Formula II-1 is typically carried out under heat, for example, with a reaction temperature ranging from about 50° C.-100° C. In some embodiments, the olefin forming reaction between the olefin forming agent of Formula III-1 and the dialdehyde of Formula II-1 can be carried out in two steps, the first step will include the nucleophilic addition of the aldehyde with the olefin forming agent to form an intermediate, which is then followed by elimination to form a double bond, for example, triphenylphosphine oxide is eliminated when the olefin forming agent is a compound of Formula III-3-A. Thus, in some embodiments, the olefin forming reaction between the olefin forming agent of Formula III-1 and the dialdehyde of Formula II-1 can be carried out initially at a lower temperature to allow the first step to take place, for example, the reaction temperature can be about 45° C. to about 65° C., such as about 60° C., for a first period of time ranging from about 1 hour to about 6 hours, such as about 2, 3, 4, or 5 hours; and subsequently, the reaction temperature can be raised to about 70° C. to about 100° C., such as about 80° C., for a second period of time of ranging from about 10 hours to about 48 hours, such as about 12, 14, 16, 18, 20, or 24 hours. Typically, the time needed for each step can be monitored, for example, by monitoring the disappearance of starting material or intermediate.

The olefin forming reaction between the olefin forming agent of Formula III-1 and the dialdehyde of Formula II-1 herein is typically carried out in the presence of a base such as an inorganic base. For example, in some embodiments, the olefin forming reaction is carried out in the presence of a carbonate base, e.g., sodium carbonate, potassium carbonate, sodium bicarbonate, potassium bicarbonate, etc. The amount of base is typically in excess of the stoichiometric amount, for example, about 30-50% in excess or 100% in excess or more.

The olefin forming reaction between the olefin forming agent of Formula III-1 and the dialdehyde of Formula II-1 typically provides an intermediate compound of Formula IV-1,

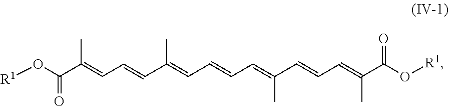

which is typically isolated in a substantially pure form. In some embodiments, the method is used for preparing a batch of the substantially pure intermediate of Formula IV-1, which can include more than 500 grams, such as more than 1 kg, more than 2 kg, more than 5 kg, more than 10 kg, or more than 100 kg of the substantially pure intermediate of Formula IV-1. In some embodiments, the batch can have about 500 grams, about 1 kg, about 2 kg, about 5 kg, about 10 kg, about 20 kg, about 50 kg, about 100 kg, of the substantially pure intermediate of Formula IV-1, or any ranges between the recited values, such as about 500 g to 5 kg, about 1-10 kg, about 5-50 kg, etc. In some embodiments, the substantially pure form of compound of Formula IV-1 is characterized as having less than 3% of other geometric isomers (total amount, other than the isomer drawn in Formula IV-1), by HPLC area percentage with detection wavelength at 254 nm and 420 nm, preferably, less than 2% such as less than 1.5%, or less than 1% of other geometric isomers. In some embodiments, the compound of Formula IV-1 can be isolated in a purity of greater than 95%, preferably, greater than 97%, greater than 98%, greater than 99%, by HPLC area percentage with detection wavelength at 254 nm and 420 nm. In some embodiments, the purity of the compound of Formula IV-1 can be up to about 99.5% or up to about 99.9%. In some embodiments, the compound of Formula IV-1 can also be characterized as having a purity of about 95%, about 97%, about 98%, about 99%, about 99.5%, about 99.9%, or any range between the recited value, such as about 95-98% or about 97-99%, by HPLC area percentage with detection wavelength at 254 nm and 420 nm. In some embodiments, the substantially pure intermediate of Formula IV-1 can also be characterized as having no greater than 0.8% such as having no detectable amount of triphenylphosphine oxide by HPLC area percentage with detection wavelength at 254 nm and 420 nm. In some embodiments, the substantially pure intermediate of Formula IV-1 can also be characterized as having no greater than 0.8% such as having no detectable amount of C10-trans dial ((2E,4E,6E)-2,7-dimethylocta-2,4,6-trienedial) by HPLC area percentage with detection wavelength at 254 nm and 420 nm. In some embodiments, the substantially pure intermediate of Formula IV-1 can also be characterized as having no greater than 0.8% such as having no detectable amount of C5-Ph₃PBr

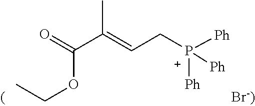 [SMILES: CCOC(=O)/C(C)=C/C[P+](c1ccccc1)(c1ccccc1)c1ccccc1]

by HPLC area percentage with detection wavelength at 254 nm and 420 nm. In some embodiments, the HPLC purities of the intermediate of Formula IV-1 can be determined following the HPLC method described herein in the Examples section (Step 3).

By using acetonitrile as a solvent, it was unexpectedly found that the isolation of the intermediate compound of Formula IV-1 can be carried out relatively easily in a way more suited for large scale synthesis. As noted in U.S. Pat. No. 8,030,350, the transdiester prepared therein typically contains a large amount of unwanted triphenylphosphine oxide and certain levels of cis isomers. On the other hand, as shown in the Examples section herein, the corresponding intermediate compound of Formula IV-1 (R¹ is ethyl) can be isolated in large scale with essentially no triphenylphine oxide or cis isomers as evidenced by HPLC analysis.

In some embodiments, isolating the intermediate compound of Formula IV-1 for the methods herein comprises cooling the reaction mixture (i.e., the olefin forming reaction mixture between the olefin forming agent of Formula III-1 and the dialdehyde of Formula II-1), filtering the reaction mixture, washing the filtered solids with acetonitrile and water, and removing residue phosphine oxide through slurring the washed solids in an alcoholic solvent (e.g., methanol, ethanol, etc.). In some embodiments, the solids obtained after the slurring step can be optionally redissolved in a suitable organic solvent (such as methylene chloride) and washed with water. This typically is done to ensure that all potassium carbonate is removed from the diesters. In some embodiments, the isolating can be substantially in accordance with the exemplified procedure herein. Although not preferred, in some embodiments, it is also contemplated that the olefin forming reaction is carried out in any solvent system suitable, but the isolation of the intermediate compound of Formula IV-1 comprises using acetonitrile optionally in combination with toluene to precipitate the trans diester and subsequently washing the precipitated solids.

In some embodiments, either one or both R¹⁰ in Formula I-1 is hydrogen or a counterion, such as a monovalent counterion (e.g., Li⁺, Na⁺, or K⁺) or a divalent counterion (e.g., Mg²⁺, Ca²⁺, etc.). In such embodiments, the method herein further comprises hydrolyzing the intermediate compound of Formula IV-1, e.g., with an alkali hydroxide, such as LiOH, NaOH, or KOH, to provide the compound of Formula I-1, wherein one or both R¹⁰ is hydrogen or a counterion. The hydrolyzing is typically carried out in an alcoholic solvent (e.g., ethanol) under heat in the presence of an alkali hydroxide base, such as LiOH, NaOH, or KOH, typically in aqueous solution. In cases where Q is Li⁺, Na⁺, or K⁺, the carotenoid salt can be isolated directly from the hydrolysis reaction using LiOH, NaOH, or KOH, respectively, without converting it into the free acid first. In some embodiments, the carotenoid salt can also be prepared from the free acid form by reacting with an appropriate base. Although base is typically used for the hydrolysis for the methods herein, in some embodiments, an acid hydrolysis is also feasible, for example, when the ester formed is a tert-butyl ester. In some embodiments, the method further comprises isolating the compound of Formula I-1 from the hydrolysis reaction mixture, e.g., through filtering the solid formed in the reaction mixture, washing the solid with water, washing the solid with an alcoholic solvent (e.g., ethanol), and drying the solid.

In some embodiments, both R¹⁰ in Formula I-1 are Na⁺, i.e., the crocetin of Formula I-1 is trans sodium crocetinate or TSC. In some embodiments, the method herein further comprises hydrolyzing the intermediate compound of Formula IV-1 with NaOH (e.g., aqueous NaOH) in an alcoholic solvent, such as ethanol. The reaction is typically carried out under heat. An exemplary procedure is provided in the Examples section. In some embodiments, the method further comprises isolating TSC from the hydrolysis reaction mixture, e.g., through filtering the solid formed in the hydrolysis reaction mixture, washing the solid with water, washing the solid with an alcoholic solvent (e.g., ethanol), and drying the solid.

The TSC prepared by the methods herein is typically isolated as a substantially pure TSC. In some embodiments, the substantially pure TSC can be characterized by having less than 3% of other geometric isomers by HPLC area percentage with detection wavelength at 254 nm and 420 nm, preferably, less than 2% such as less than 1.5%, or less than 1% of other geometric isomers. In some embodiments, the other geometric isomers are not detectable by the analytical methods described herein. In some embodiments, the other geometric isomers are in an amount of about 0.01%, about 0.05%, about 0.1%, about 0.2%, about 0.3%, about 0.4%, about 0.5%, about 0.6%, about 0.7%, about 0.8%, about 0.9%, about 1%, or any ranges between the recited values, such as about 0.1-1%, about 0.01-0.8%, about 0.1-0.8%, about 0.3-0.8%, etc. by HPLC area percentage with detection wavelength at 254 nm and 420 nm. In some embodiments, the substantially pure TSC can also be characterized as having a purity of greater than 95%, preferably, greater than 97%, greater than 98%, greater than 99%, by HPLC area percentage with detection wavelength at 254 nm and 420 nm. In some embodiments, the purity of TSC can be up to about 99.5% or up to about 99.9%. In some embodiments, the substantially pure TSC can also be characterized as having a purity of about 95%, about 97%, about 98%, about 98.5%, about 99%, about 99.5%, about 99.9%, or any range between the recited value, such as about 98-99.5%, about 99-99.5% or about 98.5-99.5%, by HPLC area percentage with detection wavelength at 254 nm and 420 nm. In some embodiments, the substantially pure TSC can also be characterized as having 1) no single unknown impurity greater than 0.8%; and/or 2) total amount of impurities not greater than 2% (such as not greater than 1%), by HPLC area percentage with detection wavelength at 254 nm and 420 nm. In some embodiments, the substantially pure TSC can also be characterized as having no greater than 0.8% such as having no detectable amount of triphenylphosphine oxide, which has a relative retention time to TSC of 0.64. In some embodiments, the substantially pure TSC can also be characterized as having no greater than 0.8% such as having no detectable amount of C20-trans diester (trans diethyl crocenate, with a chemical name of diethyl (2E,4E,6E,8E,10E,12E,14E)-2,6,11,15-tetramethylhexadeca-2,4,6,8,10,12,14-heptaenedioate), which has a relative retention time to TSC of 1.78. In some embodiments, the substantially pure TSC can also be characterized as having no greater than 0.8% such as having no detectable amount of C10-trans dial ((2E,4E,6E)-2,7-dimethylocta-2,4,6-trienedial), which has a relative retention time to TSC of 0.51. In some embodiments, the substantially pure TSC can also be characterized as having no greater than 0.8% such as having no detectable amount of C5-Ph₃PBr

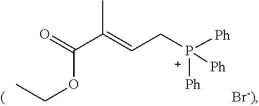 [SMILES: CCOC(=O)/C(C)=C/C[P+](c1ccccc1)(c1ccccc1)c1ccccc1]

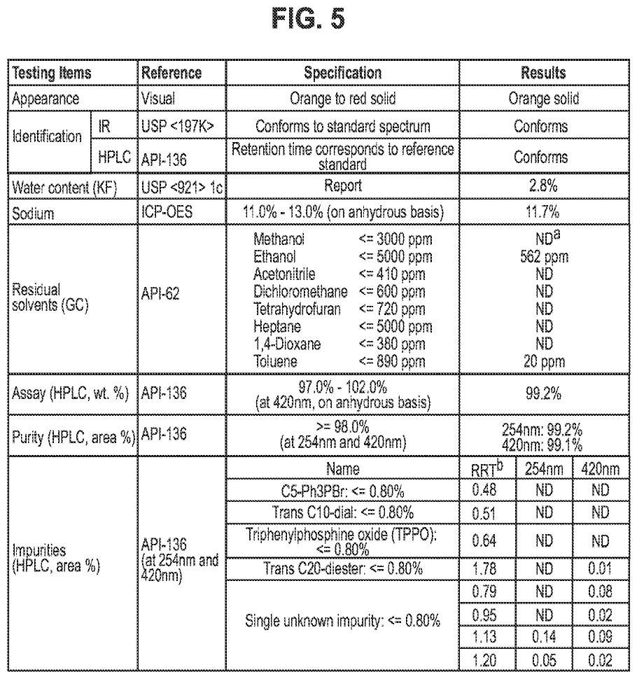

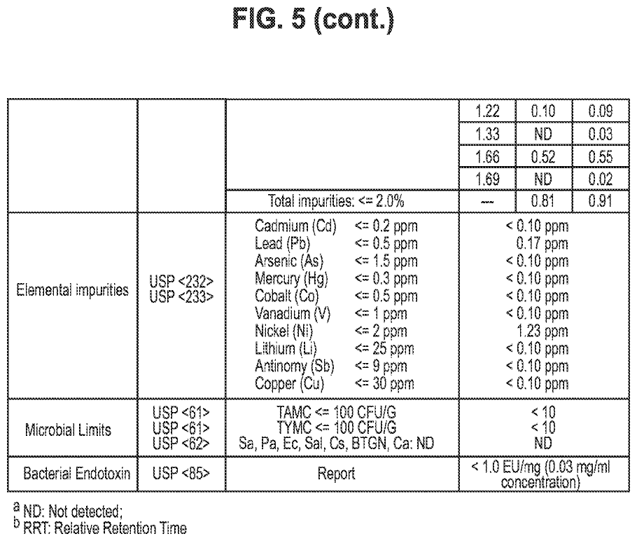

which has a relative retention time to TSC of 0.48. In some embodiments, the substantially pure TSC can also be characterized as having a purity of about 95%, about 97%, about 98%, about 98.5%, about 99%, about 99.5%, about 99.9%, or any range between the recited value, such as about 98-99.5%, about 99-99.5% or about 98.5-99.5%, by weight on anhydrous basis, as measured by HPLC with detection wavelength at 420 nm. In some embodiments, the substantially pure TSC can also have a purity characterized by a sodium content, on anhydrous basis, substantially the same as (e.g., within 80-125% of) the theoretical content of sodium calculated based on the molecular formula of TSC, as determined by Inductively Coupled Plasma Optical Emission Spectrometry (ICP-OES). In some embodiments, the substantially pure TSC can also have a purity characterized by a sodium content, on anhydrous basis, of about 11% to about 13%, as determined by Inductively Coupled Plasma Optical Emission Spectrometry (ICP-OES). Typically, the substantially pure TSC can have a low moisture content, such as less than 5%, less than 3%, etc. as determined by Karl Fischer method. In some embodiments, the substantially pure TSC also has a low residual solvent, e.g., for any single residual solvent such as ethanol or toluene, with less than 1000 ppm, or lower, or not detectable. In some embodiments, the substantially pure TSC also has low elemental impurities, such as having low levels (e.g., within specification described in the Examples section) of Cd, Pb, As, Hg, Co, V, Ni, Li, Sb, Cu, etc. as determined pursuant to USP 232. In some embodiments, the substantially pure TSC is also substantially free of microbial as determined pursuant to USP 61 or 62, with total aerobic microbial count less than 100 CFU/g and total yeasts and molds count less than 100 CFU/g, and with no detectable amount of Staphylococcus aureus, Pseudomonas aeruginosa, Escherichia coli, Salmonella enterica, Candida albicans, Clostridia, and Bile tolerant Gram Negative Bacteria. In some embodiments, the substantially pure TSC is also substantially free of bacterial endotoxin content as determined pursuant to USP 85, e.g., with less than 1 EU/mg (0.03 mg/ml concentration). In some embodiments, the substantially pure TSC is within the specification shown in FIG. 5. In some embodiments, the substantially pure TSC can also have a purity profile substantially the same as the TSC obtained from Example 1 and analyzed in the Examples section. Unless otherwise specified, the HPLC purity of TSC described herein should be understood as measured by a method in accordance with the HPLC method described in Example 1 for TSC.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4th ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wis., USA), Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7th Edition), and Larock's Comprehensive Organic Transformations (Wiley-VCH, 1999), and any of available updates as of this filing.

### Compositions, Formulation and Administration

In some embodiments, the present disclosure also provides a substantially pure carotenoid of Formula I. For example, in some embodiments, the present disclosure provides a substantially pure crocetin of Formula I-1. In some embodiments, the present disclosure provides a pharmaceutical batch of the substantially pure crocetin of Formula I-1. The pharmaceutical batch can include more than 500 grams, such as more than 1 kg, more than 2 kg, more than 5 kg, more than 10 kg, or more than 100 kg of the substantially pure crocetin of Formula I-1. In some embodiments, the pharmaceutical batch can have about 500 grams, about 1 kg, about 2 kg, about 5 kg, about 10 kg, about 20 kg, about 50 kg, about 100 kg, of the substantially pure crocetin of Formula I-1, or any ranges between the recited values, such as about 500 g to 5 kg, about 1-10 kg, about 5-50 kg, etc. In some embodiments, the substantially pure crocetin of Formula I-1 can be characterized as having less than 3% of other geometric isomers (total amount, other than the crocetin as drawn in Formula I-1), by HPLC area percentage with detection wavelength at 254 nm and 420 nm, preferably, less than 2% such as less than 1.5%, or less than 1% of other geometric isomers. In some embodiments, the other geometric isomers are not detectable by the analytical methods described herein. In some embodiments, the other geometric isomers are in an amount of about 0.01%, about 0.05%, about 0.1%, about 0.2%, about 0.3%, about 0.4%, about 0.5%, about 0.6%, about 0.7%, about 0.8%, about 0.9%, about 1%, or any ranges between the recited values, such as about 0.1-1%, about 0.01-0.8%, about 0.1-0.8%, about 0.3-0.8%, etc. by HPLC area percentage with detection wavelength at 254 nm and 420 nm. In some embodiments, the substantially pure crocetin of Formula I-1 can also be characterized as having a purity of greater than 95%, preferably, greater than 97%, greater than 98%, greater than 99%, by HPLC area percentage with detection wavelength at 254 nm and 420 nm. In some embodiments, the purity can be up to about 99.5% or up to about 99.9%. In some embodiments, the substantially pure crocetin of Formula I-1 can also be characterized as having a purity of about 95%, about 97%, about 98%, about 98.5%, about 99%, about 99.5%, about 99.9%, or any range between the recited value, such as about 98.5-99.5% or about 99-99.9%, by HPLC area percentage with detection wavelength at 254 nm and 420 nm. In some embodiments, the substantially pure crocetin of Formula I-1 can also be characterized as having a purity of about 95%, about 97%, about 98%, about 98.5%, about 99%, about 99.5%, about 99.9%, or any range between the recited value, such as about 98.5-99.5% or about 99-99.9%, by weight on anhydrous basis, as measured by HPLC with detection wavelength at 420 nm.

In some embodiments, the present disclosure also provides a substantially pure TSC. In some embodiments, the present disclosure provides a pharmaceutical batch of the substantially pure TSC. The pharmaceutical batch can include more than 500 grams, such as more than 1 kg, more than 2 kg, more than 5 kg, more than 10 kg, or more than 100 kg of the substantially pure TSC. In some embodiments, the pharmaceutical batch can have about 500 grams, about 1 kg, about 2 kg, about 5 kg, about 10 kg, about 20 kg, about 50 kg, about 100 kg, of the substantially pure TSC, or any ranges between the recited values, such as about 500 g to 5 kg, about 1-10 kg, about 5-50 kg, etc. In some embodiments, the substantially pure TSC can be characterized by having less than 3% of other geometric isomers by HPLC area percentage with detection wavelength at 254 nm and 420 nm, preferably, less than 2% such as less than 1.5%, or less than 1% of other geometric isomers. In some embodiments, the other geometric isomers are not detectable by the analytical methods described herein. In some embodiments, the other geometric isomers are in an amount of about 0.01%, about 0.05%, about 0.1%, about 0.2%, about 0.3%, about 0.4%, about 0.5%, about 0.6%, about 0.7%, about 0.8%, about 0.9%, about 1%, or any ranges between the recited values, such as about 0.1-1%, about 0.01-0.8%, about 0.1-0.8%, about 0.3-0.8%, etc. by HPLC area percentage with detection wavelength at 254 nm and 420 nm. In some embodiments, the substantially pure TSC can also be characterized as having a purity of greater than 95%, preferably, greater than 97%, greater than 98%, greater than 99%, by HPLC area percentage with detection wavelength at 254 nm and 420 nm. In some embodiments, the purity of TSC can be up to about 99.5% or up to about 99.9%. In some embodiments, the substantially pure TSC can also be characterized as having a purity of about 95%, about 97%, about 98%, about 98.5%, about 99%, about 99.5%, about 99.9%, or any range between the recited value, such as about 98-99.5%, about 99-99.5% or about 98.5-99.5%, by HPLC area percentage with detection wavelength at 254 nm and 420 nm. In some embodiments, the substantially pure TSC can also be characterized as having 1) no single unknown impurity greater than 0.8%; and/or 2) total amount of impurities not greater than 2% (such as not greater than 1%), by HPLC area percentage with detection wavelength at 254 nm and 420 nm. In some embodiments, the substantially pure TSC can also be characterized as having no greater than 0.8% such as having no detectable amount of triphenylphosphine oxide (which has a relative retention time to TSC of 0.64). In some embodiments, the substantially pure TSC can also be characterized as having no greater than 0.8% such as having no detectable amount of C20-trans diester (trans diethyl crocenate, with a chemical name of diethyl (2E,4E,6E,8E,10E,12E,14E)-2,6,11,15-tetramethylhexadeca-2,4,6,8,10,12,14-heptaenedioate), which has a relative retention time to TSC of 1.78. In some embodiments, the substantially pure TSC can also be characterized as having no greater than 0.8% such as having no detectable amount of C10-trans dial ((2E,4E,6E)-2,7-dimethylocta-2,4,6-trienedial), which has a relative retention time to TSC of 0.51. In some embodiments, the substantially pure TSC can also be characterized as having no greater than 0.8% such as having no detectable amount of C5-Ph₃PBr

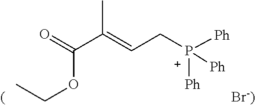 [SMILES: CCOC(=O)/C(C)=C/C[P+](c1ccccc1)(c1ccccc1)c1ccccc1]

which has a relative retention time to TSC of 0.48. In some embodiments, the substantially pure TSC can also be characterized as having a purity of about 95%, about 97%, about 98%, about 98.5%, about 99%, about 99.5%, about 99.9%, or any range between the recited value, such as about 98-99.5%, about 99-99.5% or about 98.5-99.5%, by weight on anhydrous basis, as measured by HPLC. In some embodiments, the substantially pure TSC can also have a purity characterized by a sodium content, on anhydrous basis, substantially the same as (e.g., within 80-125% of) the theoretical content of sodium calculated based on the molecular formula of TSC, as determined by Inductively Coupled Plasma Optical Emission Spectrometry (ICP-OES). In some embodiments, the substantially pure TSC can also have a purity characterized by a sodium content, on anhydrous basis, of about 11% to about 13%, as determined by Inductively Coupled Plasma Optical Emission Spectrometry (ICP-OES). Typically, the substantially pure TSC can have a low moisture content, such as less than 5%, less than 3%, etc. as determined by Karl Fischer method. In some embodiments, the substantially pure TSC also has a low residual solvent, e.g., for any single residue solvent such as ethanol or toluene, with less than 1000 ppm, or lower, or not detectable. In some embodiments, the substantially pure TSC also has low elemental impurities, such as having low levels (e.g., within specification described in the Examples section) of Cd, Pb, As, Hg, Co, V, Ni, Li, Sb, Cu, etc. as determined pursuant to USP 232. In some embodiments, the substantially pure TSC is also substantially free of microbial as determined pursuant to USP 61 or 62, with total aerobic microbial count less than 100 CFU/g and total yeasts and molds count less than 100 CFU/g, and with no detectable amount of Staphylococcus aureus, Pseudomonas aeruginosa, Escherichia coli, Salmonella enterica, Candida albicans, Clostridia, and Bile tolerant Gram Negative Bacteria. In some embodiments, the substantially pure TSC is also substantially free of bacterial endotoxin content as determined pursuant to USP 85, e.g., with less than 1 EU/mg (0.03 mg/ml concentration). In some embodiments, the substantially pure TSC is within the specification shown in FIG. 5. In some embodiments, the substantially pure TSC can also have a purity profile substantially the same as the TSC obtained from Example 1 and analyzed in the Examples section. Unless otherwise specified, the HPLC purity of TSC described herein should be understood as measured by a method in accordance with the HPLC method described in Example 1 for TSC.

Any of the carotenoid of Formula I (e.g., Formula I-1, I-2, such as those described in [54]-[56] in the Brief Summary or any of the substantially pure TSC described herein) of the present disclosure can be formulated in whole or in part as pharmaceutical compositions. Typically, the carotenoid of Formula I (e.g., the crocetin of Formula I-1) can be formulated into a pharmaceutical composition, preferably an aqueous composition, by mixing it with one or more pharmaceutically acceptable excipient or carrier. In some embodiments, the one or more pharmaceutically acceptable excipient or carrier comprises a polyethylene glycol (PEG) having a molecular weight of 200-700 Da, e.g., PEG-200, PEG-300, PEG-400, PEG-500, or PEG-600, etc. It was found that the use of low molecular weight polyethylene glycol can provide unexpected advantages in formulating a carotenoid such as TSC. Details associated with formulation with such PEGs are described in U.S. Appl. No. 63/007,878, filed Apr. 9, 2020, the content of which is herein incorporated by reference in its entirety.

Pharmaceutical compositions may include one or more nanoparticle compositions. For example, a pharmaceutical composition may include one or more nanoparticle compositions including one or more different therapeutic and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

In some embodiments, the disclosure provides an aqueous solution or pharmaceutical composition and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions provided herein (See, e.g., Remington's Pharmaceutical Sciences, 17th ed., 1989).

The provided compositions may be sterilized by conventional, known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions. In some embodiments, the pharmaceutical composition comprises a tonicity agent at a concentration of greater than 0.1%, or a concentration of 0.3% to 2.5%, 0.5% to 2.0%, 0.5% to 1.5%, 0.5% to 1.5%, 0.6% to 1.1%, or any range therein between. In some embodiments, the pharmaceutical composition comprises a tonicity agent such as dextrose, mannitol, glycerin, potassium chloride, or sodium chloride. In further embodiments, the pharmaceutical composition comprises dextrose, mannitol, glycerin, potassium chloride, or sodium chloride at a concentration of greater than 0.1%, or a concentration of 0.3% to 2.5%, 0.5% to 2.0%, 0.5% to 1.5%, 0.5% to 1.5%, 0.6% to 1.1%, or any range therein between.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In some embodiments, the provided pharmaceutical compositions are administered, for example, by intravenous infusion, topically, intraperitoneally, intravesically, or intrathecally. In particular embodiments, the pharmaceutical compositions are parentally or intravenously administered. Preferably, the pharmaceutical compositions are administered parentally, i.e. intraarticularly, intravenously, subcutaneously, or intramuscularly. In other embodiments, the pharmaceutical preparation may be administered topically.

In some embodiments, the provided pharmaceutical compositions are presented in unit-dose or multi-dose sealed containers, such as ampoules and vials.

In some embodiments, the pharmaceutical preparations are administered in unit dosage form. In such form the preparation is subdivided into unit doses containing appropriate quantities of the active component, e.g., a liposome composition and aqueous solution. The unit dosage form can be a packaged preparation, the package containing discrete quantities of preparation. The composition can, if desired, also contain other compatible therapeutic agents (e.g., as described herein).

In some embodiments, the pharmaceutical compositions provided herein can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the pharmaceutical composition being employed. For example, dosages can be empirically determined considering the type and stage of the disease, disorder or condition diagnosed in a particular patient. The dose administered to a patient, in the context of the provided pharmaceutical compositions (e.g., liposome compositions) should be sufficient to affect a beneficial therapeutic response in the patient over time. The size of the dose will also be determined by the existence, nature, and extent of any adverse side-effects that accompany the administration of a particular pharmaceutical composition in a particular patient. Determination of the proper dosage for a particular situation is within the skill of the practitioner. Generally, treatment is initiated with smaller dosages which are less than the optimum dose of the pharmaceutical composition. Thereafter, the dosage is increased by small increments until the optimum effect under circumstances is reached. For convenience, the total daily dosage may be divided and administered in portions during the day, if desired.

### Liposome Loading

In additional embodiments, the disclosure provides an active loading method to generate an ionizable carotenoid salt inside a liposome formulation using a metal salt or pH gradient.

In some embodiments, the disclosure provides a method of preparing a loaded liposomal formulation comprising an ionizable carotenoid salt inside a liposome using a soluble metal salt gradient, wherein the method comprises:
(a) mixing an ionizable carotenoid of Formula I (e.g., Formula I-1, I-2, such as those described in [54]-[56] in the Brief Summary or any of the substantially pure TSC described herein) with a liposomal solution containing liposomes comprising a weak acid salt of a multivalent metal; and
(b) maintaining the mixture of (a) for a sufficient time to load carotenoid into liposomes.

Loading of the ionizable carotenoid of Formula I may be established by maintaining the ionizable carotenoid in the liposomal solution for a suitable amount of time at a suitable temperature. Depending on the composition of the liposome, and the temperature, pH, and chemical nature of the ionizable carotenoid, loading of the ionizable carotenoid may occur over a time period of minutes or hours. In some embodiments, loading is carried out at temperatures of, for example, 0° C. to 95° C., or 20° C. to 75° C., or any range therein, preferably from about 40° C. to about 80° C.

In some embodiments, the ionizable carotenoid is an ionizable carotenoid in an aqueous solutions (e.g., trans-crocetin and trans-norbixin). In some embodiments, the weak acid is selected from acetic acid, gluconic acid, tartaric acid, glutamic acid, citric acid, formic acid, and glycinic acid. In some embodiments, the weak acid salt of a multivalent metal is used at a concentration from 0 mM to 2000 mM, or 50 mM to 500 mM, or any range therein between. In some embodiments, the multivalent metal is selected from Ca²⁺, Mg²⁺, Zn²⁺, Cu²⁺, Co²⁺, Fe²⁺ and Fe³⁺. In some embodiments, the weak acid is acetic acid and the multivalent metal is Ca²⁺ (i.e., the weak acid salt of the multivalent metal is calcium acetate). In some embodiments, the weak acid is acetic acid and the multivalent metal is Mg²⁺ (i.e., the weak acid salt of the multivalent metal is magnesium acetate). Pharmaceutical compositions prepared according to the provided methods are also encompassed by the disclosure. The liposomal solution is preferably a buffered solution. However, it is appreciated that any suitable solvent may be use to prepare and use the provided compositions. A preferred liposome solution has a pH at about physiological pH and comprises a buffer which has a buffering range to include physiological pH. A non-limiting example of a suitable buffer for the liposome solution is HEPES (e.g., 5 mM HEPES buffered saline pH 6.5). Pharmaceutical compositions prepared according to the method are also encompassed by the disclosure.

In some embodiments, the liposome loading method further comprises the step of heating and cooling the drug loading mixture.

In some embodiments, the disclosure further provides the step of (c) removing unencapsulated ionizable carotenoid from the liposome preparation prepared according to (b). In some embodiment, the removal is carried out by passing the liposome preparation through a gel filtration column equilibrated with a second aqueous buffered solution, centrifugation, or dialysis, or related techniques. After removal of unencapsulated ionizable carotenoid, the extent of ionizable carotenoid loading may be determined by measurement of ionizable carotenoid and lipid levels according to conventional techniques. Lipid and drug concentrations may be determined using any suitable method known in the art, such as scintillation counting, spectrophotometric assays, and high performance liquid chromatography. Replacement of the liposome preparation solution to remove unencapsulated carotenoid and counterion, such as sodium acetate, can be accomplished using any of various techniques, known in the art, including but not limited to chromatography of the liposome preparation through an extensive gel filtration column equilibrated with a second aqueous buffered solution, by centrifugation, extensive or repeated dialysis, exchange of the liposomal preparation, treating the liposomal preparation with chelating agents or by related techniques. Pharmaceutical compositions prepared according to the provided methods are also encompassed by the disclosure.

In some embodiments, the weak acid salt used in the loading method is an organic acid (e.g., an organic acid selected from acetic acid, gluconic acid, tartaric acid, glutamic acid, citric acid, formic acid, and glycinic acid).

In some embodiments, the multivalent metal used in the loading method is a bivalent metal (e.g., a bivalent metal selected from Ca²⁺, Mg²⁺, Zn²⁺, Cu²⁺, Co²⁺, and Fe²⁺), or a trivalent metal such as Fe³⁺.

In further embodiments, the disclosure provides a method of preparing a loaded liposomal composition comprising an ionizable carotenoid salt of Formula I (e.g., Formula I-1, I-2, such as those described in [54]-[56] in the Brief Summary or any of the substantially pure TSC described herein) using a soluble acetate metal salt gradient (calcium acetate or magnesium acetate). In yet further embodiments, the loading method uses acetic acid as the weak acid and Ca²⁺ or Mg²⁺ as the bivalent metal is (i.e., the weak acid salt of the bivalent metal is calcium acetate or magnesium acetate, respectively).

In some embodiments, the disclosure further provides the step of (c) removing unencapsulated ionizable carotenoid from the liposome preparation prepared according to (b). In some embodiment, the removal is carried out by passing the liposome preparation through a gel filtration column equilibrated with a second aqueous buffered solution, centrifugation, or dialysis, or related techniques. After removal of unencapsulated ionizable carotenoid, the extent of ionizable carotenoid loading may be determined by measurement of ionizable carotenoid and lipid levels according to conventional techniques. Lipid and drug concentrations may be determined using any suitable method known in the art, such as scintillation counting, spectrophotometric assays, and high performance liquid chromatography. Replacement of the liposome preparation solution to remove unencapsulated carotenoid and counterion, such as sodium acetate, can be accomplished using any of various techniques, known in the art, including but not limited to chromatography of the liposome preparation through an extensive gel filtration column equilibrated with a second aqueous buffered solution, by centrifugation, extensive or repeated dialysis, exchange of the liposomal preparation, treating the liposomal preparation with chelating agents or by related techniques. Pharmaceutical compositions prepared according to the provided methods are also encompassed by the disclosure.

In some embodiments, the disclosure provides a method of preparing a liposomal composition containing trans-crocetin inside a liposome using a soluble metal salt gradient, wherein the method comprises:
(a) mixing a TSC (e.g., any of the substantially pure TSC described herein) with a liposomal solution containing liposomes comprising a weak acid salt of a multivalent metal; and
(b) maintaining the mixture of (a) for a sufficient time to load trans-crocetin into liposomes.

Loading of trans-crocetin may be established by maintaining the trans-crocetin in the liposomal solution for a suitable amount of time at a suitable temperature. Depending on the composition of the liposome, and the temperature, pH, and chemical nature of trans-crocetin, loading of the trans-crocetin may occur over a time period of minutes or hours. In some embodiments, loading is carried out at temperatures of, for example, 0° C. to 95° C., or 20° C. to 75° C., or any range therein, preferably from about 40° C. to about 80° C.

In some embodiments, the weak acid is selected from acetic acid, gluconic acid, tartaric acid, glutamic acid, citric acid, formic acid, and glycinic acid. In some embodiments, the weak acid salt of a multivalent metal is used at a concentration from 0 mM to 2000 mM, or 50 mM to 500 mM, or any range therein between. In some embodiments, the multivalent metal is selected from Ca²⁺, Mg²⁺, Zn²⁺, Cu²⁺, Co²⁺, Fe²⁺, and Fe³⁺. In some embodiments, the weak acid is acetic acid and the multivalent metal is Ca²⁺ (i.e., the weak acid salt of the multivalent metal is calcium acetate). In some embodiments, the weak acid is acetic acid and the multivalent metal is Mg²⁺ (i.e., the weak acid salt of the multivalent metal is magnesium acetate). Pharmaceutical compositions prepared according to the method are also encompassed by the disclosure. The liposomal solution is preferably a buffered solution. However, it is appreciated that any suitable solvent may be utilized to practice the provided compositions and methods. A preferred liposome solution has a pH at about physiological pH and comprises a buffer which has a buffering range to include physiological pH. Non-limiting example of suitable buffers for the liposome solution is 5 mM HEPES buffered saline pH 6.5. Pharmaceutical compositions prepared according to the method are also encompassed by the disclosure.

In some embodiments, the disclosure further provides the step of (c) removing unencapsulated trans-crocetin from the liposome preparation prepared according to (b). In some embodiment, the removal is carried out by passing the liposome preparation through a gel filtration column equilibrated with a second aqueous buffered solution, or by centrifugation, dialysis, or related techniques. After removal of unencapsulated trans-crocetin, the extent of trans-crocetin loading may be determined by measurement of trans-crocetin and lipid levels according to conventional techniques. Lipid and drug concentrations may be determined by employing any suitable method known in the art, such as scintillation counting, spectrophotometric assays, and high performance liquid chromatography. Replacement of the liposome preparation solution to remove unencapsulated trans-crocetin and counterion, such as sodium acetate, can be accomplished using any of various techniques, known in the art, including but not limited to chromatography of the liposome preparation through an extensive gel filtration column equilibrated with a second aqueous buffered solution, centrifugation, extensive or repeated dialysis, exchange of the liposomal preparation, treating the liposomal preparation with chelating agents or by related techniques. Pharmaceutical compositions prepared according to the provided methods are also encompassed by the disclosure.

Multivalent counterions used in accordance with the present disclosure can be encapsulated in liposomes according to techniques described herein or otherwise known in the art. These methods include, for example, passive encapsulation techniques described herein or otherwise known in the art. Loading of an ionizable carotenoid such as trans-crocetin may be established by maintaining the ionizable carotenoid in the liposomal solution for a suitable amount of time at a suitable temperature. Depending on the composition of the liposome, and the temperature, pH, and chemical nature of the ionizable carotenoid, loading of the ionizable carotenoid may occur over a time period of minutes or hours. In some embodiments, loading is carried out at temperatures of, for example, 0° C. to 95° C., or 20° C. to 75° C., or any range therein between, preferably from about 40° C. to about 80° C., or any range therein between.

The compositions and characteristics of the liposomes that can be loaded according to the provided methods is not particularly limited. The properties of liposomes are influenced by the nature of lipids used to make the liposomes. A wide variety of lipids have been used to make liposomes. These include cationic, anionic and neutral lipids. The liposomes loaded according to the provided methods may contain functionalized and/or non-functionalized lipids. In some embodiments, the liposomes comprising the carotenoid compositions (e.g., CTC and MTC) are anionic or neutral. In other embodiments, the provided liposomes are cationic. The determination of the charge (e.g., anionic, neutral or cationic) can routinely be determined by measuring the zeta potential of the liposome. The zeta potential of the liposome can be positive, zero or negative. In some embodiments, the zeta potential of the liposome is −150 to 150 mV, or −50 to 50 mV, or any range therein between. In some embodiments, the zeta potential of the liposome is less than or equal to zero. In some embodiments, the zeta potential of the liposome is −150 to 0, −50 to 0 mV, −40 to 0 mV, −30 to 0 mV, −25 to 0 mV, −20 to 0 mV, −10 to 0 mV, −9 to 0 mV, −8 to 0 mV, −7 to 0 mV, −6 to 0 mV, −5 to 0 mV, −4 to 0 mV, −3 to 0 mV, −2 to 0 mV, −1 to 0 mV, or −8 to 2 mV, or any range therein between. In other embodiments, the zeta potential of the liposome is more than zero. In some embodiments, the liposome has a zeta potential that is 0.2 to 150 mV, 1 to 50 mV, 1 to 40 mV, 1 to 30 mV, 1 to 25 mV, 1 to 20 mV, 1 to 15 mV, 1 to 10 mV, 1 to 5 mV, 2 to 10 mV, 3 to 10 mV, 4 to 10 mV, or 5 to 10 mV, or any range therein between.

In some embodiments, the liposomes loaded according the the disclosed methods include a steric stabilizer that increases their longevity in circulation. One or more steric stabilizers such as a hydrophilic polymer (polyethylene glycol (PEG)), a glycolipid (monosialo-ganglioside (GM1)) or others occupies the space immediately adjacent to the liposome surface and excludes other macromolecules from this space. Consequently, access and binding of blood plasma opsonins to the liposome surface are hindered, and thus interactions of macrophages with such liposomes, or any other clearing mechanism, are inhibited and longevity of the liposome in circulation is enhanced. In some embodiments, the steric stabilizer or the population of steric stabilizers is a PEG or a combination comprising PEG. In further embodiments, the steric stabilizer is a PEG or a combination comprising PEG with a number average molecular weight (Mn) of 200 to 5000 Daltons. These PEG(s) can be of any structure such as linear, branched, star or comb structure and are commercially available.

In some embodiments, the diameter of the liposomes loaded according the the disclosed methods have a mean diameter of for example, 20 nm to 500 nm (nanometer), or 20 nm to 200 nm, or any range therein between. In some embodiments, the liposomes have a mean diameter of 80 nm to 120 nm, or any range therein between.

Lipoosomal formulations and pharmaceutical compositions comprising loaded liposomes encapsulating an ionizable carotenoid salt prepared according to the provided loading methods are also encompassed by the disclosure. In some embodiments, the ionizable carotenoid is an ionizable carotenoid of Formula I (e.g., trans-crocetin and trans-norbixin). In some embodiments, the disclosure provides a pharmaceutical composition comprising a liposome encapsulating an ionizable carotenoid of Formula I (e.g., Formula I-1, I-2, such as those described in [54]-[56] in the Brief Summary or any of the substantially pure TSC described herein), wherein the ionizable carotenoid is loaded into liposomes in the presence of intra-liposomal multivalent counterions (e.g., Ca²⁺, Mg²⁺, Zn²⁺, Cu²⁺, Co²⁺, and Fe²⁺, and Fe³⁺). In some embodiments, the multivalent counterions comprise Ca²⁺. In some embodiments, the multivalent counterions comprise Mg²⁺. In some embodiments, the multivalent counterions comprise Fe³⁺.

In some embodiments, the disclosure provides a liposomal formulations and pharmaceutical compositions comprising loaded liposomes encapsulating trans-crocetin that are prepared according to the provided laoding methods In further embodiments, the liposomes were prepared by loading trans-crocetin in the presence of intra-liposomal multivalent counterions (e.g., Ca²⁺, Mg²⁺, Zn²⁺, Cu²⁺, Co²⁺, and Fe²⁺, and Fe³⁺). In some embodiments, the multivalent counterions comprise Ca²⁺. In some embodiments, the multivalent counterions comprise Mg²⁺. In some embodiments, the multivalent counterions comprise Fe³⁺.

### Methods of Treatment and Use

In additional embodiments, the disclosure provides a method for increasing the delivery of oxygen in a subject who has or is at risk for developing ischemia (e.g., tissue hypoperfusion), that comprises administering to the subject an effective amount of a pharmaceutical composition provided herein, such as a pharmaceutical composition of any one of [66]-[68] in the Brief Summary, thereby increasing the delivery of oxygen to the tissues and/or organs in the subject. In some embodiments, the subject has or is at risk for developing ischemia. In some embodiments, the pharmaceutical composition is administered to the subject before, during or following surgery (e.g., transplantation; reattachment of severed extremities, body parts or soft tissues; graft surgery, and vascular surgery). In some embodiments, the pharmaceutical composition is administered to a subject who has or is at risk for developing a wound, a burn injury, an electrical injury, or exposure to ionizing radiation. In some embodiments, the pharmaceutical composition is administered to a subject who has or is at risk for developing peripheral vascular disease, coronary artery disease, stroke, thrombosis, a clot, chronic vascular obstruction or vasculopathy (e.g., secondary to diabetes, hypertension, or peripheral vascular disease), or cerebral ischemia, pulmonary hypertension (adult or neonate); sickle cell disease; neointimal hyperplasia or restenosis (following angioplasty or stenting). In some embodiments, the pharmaceutical composition is administered to a subject who has or is at risk for developing a myopathy, kidney disease; asthma or adult respiratory distress syndrome; Alzheimer's and other dementias secondary to compromised cranial blood flow. In some embodiments, the method comprises administering an effective amount of the pharmaceutical composition (e.g., any one of [66]-[68] in the Brief Summary) to the subject. Use of a pharmaceutical composition provided herein (e.g., the pharmaceutical composition of any one of [66]-[68] in the Brief Summary), in the manufacture of a medicament for increasing the delivery of oxygen in a subject is also provided herein. As are, pharmaceutical compositions (e.g., any one of [66]-[68] in the Brief Summary) for use in a medical medicament.

Methods are also disclosed herein for increasing the delivery of oxygen in a neonate subject or a subject who is elderly that comprises administering to the subject an effective amount of a pharmaceutical composition provided herein (e.g., a liposomal composition), thereby increasing the delivery of oxygen to the tissues and/or organs of the subject. In some embodiments, the subject is elderly (e.g., a human subject that is more than 65, more than 70, more than 75, or more than 80 years of age). In some embodiments, the subject has or is at risk for developing a respiratory condition or disease (e.g., COPD, respiratory distress syndrome or adult respiratory distress syndrome). In some embodiments, the subject has or is at risk for developing a degenerative disorder, such as dementia or Alzheimer's disease. In some embodiments, the method comprises administering an effective amount of the pharmaceutical composition (e.g., any one of [66]-[68] in the Brief Summary) to the subject. Use of a pharmaceutical composition provided herein ((e.g., any one of [66]-[68] in the Brief Summary), in the manufacture of a medicament for increasing the delivery of oxygen in an elderly subject is also provided herein. As are, pharmaceutical compositions (e.g., any one of [66]-[68] in the Brief Summary) for use in a medical medicament.

In additional embodiments, the disclosure provides a method for increasing the delivery of oxygen in a subject who has or is at risk for developing ischemia/reperfusion injury, that comprises administering to the subject an effective amount of a pharmaceutical composition provided herein, such as liposomal a composition, thereby increasing the delivery of oxygen to the tissues and/or organs in the subject. In some embodiments, the pharmaceutical composition is administered to the subject before, during or following surgery (e.g., transplantation; reattachment of severed extremities, body parts or soft tissues; graft surgery, and vascular surgery). In some embodiments, the ischemia/reperfusion injury is due to a condition selected from infarction, atherosclerosis, thrombosis, thromboembolism, lipid-embolism, bleeding, stent, surgery, angioplasty, end of bypass during surgery, organ transplantation, total ischemia, and combinations thereof. In some embodiments, the ischemia/reperfusion injury is produced in an organ or a tissue selected from the group: heart, liver, kidney, brain, intestine, pancreas, lung, skeletal muscle and combinations thereof. In some embodiments, the ischemia/reperfusion injury is selected from the group: organ dysfunction, infarct, inflammation, oxidative damage, mitochondrial membrane potential damage, apoptosis, reperfusion-related arrhythmia, cardiac stunning, cardiac lipotoxicity, ischemia-derived scar formation, and combinations thereof. In particular embodiments, the ischemia/reperfusion injury is due to myocardial infarction. In some embodiments, the pharmaceutical composition is administered to a subject who has or is at risk for developing peripheral vascular disease, coronary artery disease, stroke, thrombosis, a clot, chronic vascular obstruction or vasculopathy (e.g., secondary to diabetes, hypertension, or peripheral vascular disease), or cerebral ischemia, pulmonary hypertension (adult or neonate); sickle cell disease; neointimal hyperplasia or restenosis (following angioplasty or stenting). In some embodiments, the pharmaceutical composition is administered to a subject who has or is at risk for developing a myopathy, kidney disease; asthma or adult respiratory distress syndrome; Alzheimer's and other dementias secondary to compromised cranial blood flow. In some embodiments, the method comprises administering an effective amount of the pharmaceutical composition (e.g., any one of [66]-[68] in the Brief Summary) to the subject. Use of a pharmaceutical composition provided herein (e.g., any one of [66]-[68] in the Brief Summary), in the manufacture of a medicament for increasing the delivery of oxygen in a subject is also provided herein. As are, pharmaceutical compositions (e.g., any one of [66]-[68] in the Brief Summary) for use in a medical medicament.

The pharmaceutical compositions provided herein such as liposomal compositions, have uses that provide advances over prior treatments of diseases and disorders that include without limitation, infection and infectious diseases such as HIV/AIDS: human immunodeficiency virus-1 (HIV-1), tuberculosis, malaria and its complications such as cerebral malaria, severe anemia, acidosis, acute kidney failure and ARDS, sepsis, inflammation (e.g., chronic inflammatory diseases), ischemia, (including an ischemic condition such as ischemic stroke, coronary artery disease, peripheral vascular disease, cerebral vascular disease, ischemia associated renal pathologies, and ischemia associated with wounds); shock (e.g., hemorrhagic shock), stroke, cardiovascular disease, renal pathologies, wound healing, metabolic disease, hyperproliferative diseases such as cancer, and disorders of the immune system, cardiovascular system, digestive, nervous, respiratory, and endocrine system. In some embodiments, the disclosure provides a method for treating or preventing a disease, disorder or condition in a subject needing such treatment or prevention, the method comprising administering an effective amount of a pharmaceutical composition administering an effective amount of a pharmaceutical composition provided herein (e.g., any one of [66]-[68] in the Brief Summary) to the subject. Use of a pharmaceutical composition provided herein (e.g., any one of [66]-[68] in the Brief Summary), in the manufacture of a medicament for the treatment of a disease, disorder or condition in a subject is also provided herein. As are, pharmaceutical compositions (e.g., any one of [66]-[68] in the Brief Summary) for use in a medical medicament.

In some embodiments, the disclosure provides a method for treating or preventing a disease, disorder or condition associated with endotoxemia in a subject needing such treatment or prevention, the method comprising administering an effective amount of a pharmaceutical composition provided herein (e.g., any one of [66]-[68] in the Brief Summary) to the subject.

In some embodiments, the disclosure provides a method for treating or preventing a disease, disorder or condition associated with sepsis in a subject needing such treatment or prevention, the method comprising administering an effective amount of a pharmaceutical composition provided herein (e.g., any one of [66]-[68] in the Brief Summary) to the subject. In some embodiments, the subject has a low grade endotoxemic disease.

In some embodiments, the disclosure provides a method for treating or preventing a subject at risk of developing sepsis, the method comprising administering an effective amount of a pharmaceutical composition provided herein ((e.g., any one of [66]-[68] in the Brief Summary) to the subject. In some embodiments, the subject is immunocompromised or immunosuppressed. In some embodiments, the subject is critically ill. In some embodiments, the subject elderly or neonatal. In some embodiments, the subject has febrile neutropenia. In some embodiments, the subject has an infection.

In some embodiments, the disclosure provides a method for treating or preventing a disease, disorder or condition associated with burn injury in a subject that is a burn victim, the method comprising administering an effective amount of a pharmaceutical composition provided herein (e.g., any one of [66]-[68] in the Brief Summary) to the subject.

In some embodiments, the disclosure provides a method for treating or preventing a disease, disorder or condition associated with infection in a subject needing such treatment or prevention, the method comprising administering an effective amount of a pharmaceutical composition provided herein (e.g., any one of [66]-[68] in the Brief Summary) to the subject. In some embodiments, the infection is a bacterial infection (e.g., a P. aeruginosa infection, an S. aureus infection (e.g., MRSA), Mycobacterium tuberculosis infection, an enterococcal infection (e.g., VRE), or a condition associated therewith. In some embodiments, the infection is a fungal infection (e.g., a candidiasis infection such as invasive candidiasis) or a condition associated therewith. In some embodiments, the infection is a parasitic infection (e.g., Schistosomiasis, and human African trypanosomiasis), or a condition associated therewith. In some embodiments, the infection is malaria or a condition associated therewith, such as cerebral malaria, severe anemia, acidosis, acute kidney failure and ARDS. In some embodiments, the infection is a viral infection (e.g., Ebola, Dengue and Marburg) or a condition associated therewith, such as influenza, measles, and a viral hemorrhagic fever.

In some embodiments, the disclosure provides a method for treating or preventing a disease, disorder or condition associated with ischemia or hypoxia in a subject needing such treatment or prevention, the method comprising administering an effective amount of a pharmaceutical composition provided herein (e.g., any one of [66]-[68] in the Brief Summary) to the subject. In some embodiments, the disease or condition associated with ischemia or hypoxia is associated with surgery or traumatic injury. In some embodiments, the disease or condition is ischemic-reperfusion injury, transient cerebral ischemia, cerebral ischemia-reperfusion, ischemic stroke, hemorrhagic stroke, traumatic brain injury, ischemic heart disease, migraine (e.g., a chronic migraine or severe migraine disorder), gastrointestinal ischemia, kidney disease, pulmonary embolism, acute respiratory failure, neonatal respiratory distress syndrome, obstetric emergencies to reduce perinatal comorbidity (such as, pre/eclampsia and conditions that lead to cerebral palsy), myocardial infarction, acute limb or mesenteric ischemia, cardiac cirrhosis, chronic peripheral vascular disease, congestive heart failure, atherosclerotic stenosis, anemia, thrombosis, embolism, macular degeneration, a neurodegenerative disease (e.g., Alzheimer's disease, Parkinson's disease, and Amyotrophic Lateral Sclerosis (ALS)), sleep apnea, and surgery or traumatic injury. In some embodiments, the disease or condition associated with ischemia or hypoxia is myocardial infarction, or congestive heart failure with or without cardiac cirrhosis. In some embodiments, the disease or condition is pulmonary embolism, acute respiratory failure, chronic peripheral vascular disease, atherosclerotic stenosis, anemia, thrombosis, or embolism. In some embodiments, the disease or condition associated with ischemia or hypoxia is macular degeneration or an oncologic condition associated with hypoxia. In some embodiments, the disease or condition is kidney disease. In some embodiments, the disease or condition is lipopolysaccharide medication or toxin induced acute kidney injury (AKI) or end stage kidney disease.

In some embodiments, the disclosure provides a method for treating or preventing a disease, disorder or condition associated with shock in a subject needing such treatment or prevention, the method comprising administering an effective amount of a pharmaceutical composition provided herein (e.g., any one of [66]-[68] in the Brief Summary) to the subject. In some embodiments, the disease or condition is associated with cardiogenic shock. In some embodiments, the disease or condition is associated with, hypovolemic shock. In some embodiments, the disease or condition is associated with septic shock or other forms of distributive shock. In some embodiments, the disease or condition is associated with neurogenic shock. In some embodiments, the disease or condition is associated with anaphylactic shock.

In some embodiments, the disclosure provides a method for treating or preventing a disease, disorder or condition associated with nitric oxide deficiency in a subject needing such treatment or prevention, the method comprising administering an effective amount of a pharmaceutical composition provided herein (e.g., any one of [66]-[68] in the Brief Summary) to the subject. In some embodiments, the disease or disorder is sickle cell disease, paroxysmal nocturnal hemoglobinuria (PNH), a hemolytic anemia, a thalassemia, another red blood cell disorder, or a condition associated therewith. In some embodiments, the disease or disorder is a purpura such as thrombotic thrombocytic purpura (TTP), hemolytic uremic syndrome (HUS), idiopathic thrombocytopenia (ITP), or and another platelet disorder, or a condition associated therewith. In some embodiment, the disease or disorder is a coagulation abnormality such as disseminated intravascular coagulopathy (DIC), purpura fulminans, heparin induced thrombocytopenia (HIT), hyperleukocytosis, hyper viscosity syndrome, or a condition associated therewith.

In some embodiments, the disclosure provides a method for treating or preventing a disease, disorder or condition associated with inflammation in a subject needing such treatment or prevention, the method comprising administering an effective amount of a pharmaceutical composition provided herein (e.g., any one of [66]-[68] in the Brief Summary) to the subject. In some embodiments, the disease or condition associated with inflammation is low-grade inflammation. In some embodiments, the disease or condition associated with inflammation is systemic inflammation. In some embodiments, the disease or condition associated with inflammation is acute inflammation or a chronic inflammatory disease.

In some embodiments, the disclosure provides a method for treating or preventing a disease, disorder or condition associated with a cardiovascular disease or condition in a subject needing such treatment or prevention, the method comprising administering an effective amount of a pharmaceutical composition provided herein (e.g., any one of [66]-[68] in the Brief Summary) to the subject. In some embodiments, cardiovascular disease or condition is coronary artery disease. In some embodiments the cardiovascular disease or condition is myocardial infarction, sudden cardiac death, cardiorespiratory arrest, hypertension, pulmonary arterial hypertension, atherosclerosis, occlusive arterial disease, Raynaud's disease, peripheral vascular disease, other vasculopathies such as Buerger's disease, Takayasu's arthritis, and post-cardiac arrest syndrome (PCAS), chronic venous insufficiency, heart disease, congestive heart failure, or a chronic skin ulcer. Methods and biomarkers for evaluating cardiovascular health (e.g., levels of conventional troponins (cTnI and cTnT), Ischemia-Modified Albumin (IMA), B-type Natriuretic Peptide and N-terminal proBNP, whole blood choline, and unesterified free fatty acid (FFAu)) and cardiovascular injury and disease, and the efficacy of treatment regimens are known in the art

In some embodiments, the disclosure provides a method for treating or preventing a disease, disorder or condition associated with a liver disease, injury or condition in a subject needing such treatment or prevention, the method comprising administering an effective amount of a pharmaceutical composition provided herein (e.g., any one of [66]-[68] in the Brief Summary) to the subject. In some embodiments, the liver disease or condition is hepatic ischemia/reperfusion injury. In some embodiments, the liver disease or condition is a hepatic resection or liver transplantation. In some embodiments, the liver disease or condition is cirrhosis. In some embodiments, the liver disease or condition is nonalcoholic fatty liver disease (NAFLD), non-alcoholic steatohepatitis (NASH). In some embodiments, the liver disease or condition is alcoholic liver disease. In some embodiments, the liver disease or condition is acute liver injury. Methods and biomarkers for evaluating liver health (e.g., levels of liver enzymes ALT, AST, ALP, and LDH), as well as liver injury and disease and the efficacy of treatment regimens are known in the art.

In some embodiments, the disclosure provides a method for treating or preventing a disease, disorder or condition associated with a lung disease or condition in a subject needing such treatment or prevention, the method comprising administering an effective amount of a pharmaceutical composition provided herein (e.g., any one of [66]-[68] in the Brief Summary) to the subject. In some embodiments, the lung disease or condition is acute respiratory distress syndrome (ARDS). In some embodiments, the lung disease or condition is chronic obstructive pulmonary disease. In some embodiments, the lung disease or condition is pulmonary fibrosis. In some embodiments, the lung disease or condition is pulmonary hemorrhage. In some embodiments, the lung disease or condition is asthma. In some embodiments, the lung disease or condition is lung injury. In some embodiments, the lung disease or condition is lung cancer. In some embodiments, the condition is cystic fibrosis.

In some embodiments, the disclosure provides a method for treating or preventing a disease, disorder or condition associated with a kidney disease or condition in a subject needing such treatment or prevention, the method comprising administering an effective amount of a pharmaceutical composition provided herein (e.g., any one of [66]-[68] in the Brief Summary) to the subject. In some embodiments, the kidney disease or condition is lipopolysaccharide-induced acute kidney injury (AKI). In some embodiments, the kidney disease or condition is chronic renal failure with or without end stage kidney disease. Methods and biomarkers for evaluating renal health (e.g., levels of N-acetyl-p-glucosaminidase (NAG), ui-microglobulin (uiM), Cystatin-C(Cys-C), Retinol binding protein (RBP), microalbumin, Kidney injury molecule-1 (KIM-1), Clusterin, Interleukin-18 (IL-18), Cysteine-rich protein (Cyr61), osteopontin (OPN), Fatty acid-binding protein (FABP), Fetuin-A, and neutrophil gelatinase-associated lipocalin (NGAL)), as well as renal injury and disease and the efficacy of treatment regimens are known in the art.

In some embodiments, the disclosure provides a method for treating or preventing a disease, disorder or condition associated with a vascular disease in a subject needing such treatment or prevention, the method comprising administering an effective amount of a pharmaceutical composition provided herein (e.g., any one of [66]-[68] in the Brief Summary) to the subject. In some embodiments, the disease or condition is coronary artery disease. In some embodiments, the disease or condition is hypertension. In some embodiments, the disease or condition is atherosclerosis. In some embodiments, the disease or condition is post-cardiac arrest syndrome (PCAS). In some embodiments, the disease or condition is occlusive arterial disease, peripheral vascular disease, chronic venous insufficiency, chronic skin ulcers, or Raynaud's disease. In some embodiments, the disease, disorder or condition associated with a vascular disease is heart disease. In further embodiments, the disease, disorder or condition is congestive heart failure. In some embodiments, the disease, disorder or condition associated with vascular disease is ischemic bowel disease.

In some embodiments, the disclosure provides a method for treating or preventing a disease, disorder or condition associated with a heart attack or stroke in a subject needing such treatment or prevention and/or at risk of having a heart attack or stroke, the method comprising administering an effective amount of a pharmaceutical composition provided herein (e.g., any one of [66]-[68] in the Brief Summary) to the subject. In some embodiments, the disease, disorder or condition is ischemic stroke. In some embodiments, the disease, disorder or condition is hemorrhagic stroke. Methods and biomarkers for evaluating heart attack and stroke (e.g., levels of blood B-type natriuretic peptide (BNP), C-reactive protein (CRP), GlycA, CK-MB, Cardiac troponin, myoglobin, low-density lipoprotein-cholesterol and hemoglobin A1c (HgA1c), lipoprotein-associated phospholipase A2, glial fibrillary acidic protein, S100b, neuron-specific enolase, myelin basic protein, interleukin-6, matrix metalloproteinase (MMP)-9, D-dimer, and fibrinogen)), and the efficacy of treatment regimens are known in the art.

In some embodiments, the disclosure provides a method for treating or preventing a disease, disorder or condition associated with nervous system in a subject needing such treatment or prevention, the method comprising administering an effective amount of a pharmaceutical composition provided herein (e.g., any one of [66]-[68] in the Brief Summary) to the subject. In some embodiments, the disease or condition is pain (e.g., chronic pain). In some embodiments, the disease or condition is a neurodegenerative disease (e.g., Alzheimer's disease or Parkinson's disease).

In some embodiments, the disclosure provides a method for treating or preventing a disease, disorder or condition associated with inflammatory bowel disease in a subject needing such treatment or prevention, the method comprising administering an effective amount of a pharmaceutical composition provided herein (e.g., any one of [66]-[68] in the Brief Summary) to the subject. In some embodiments, the disease, disorder or condition is Crohn's disease. In some embodiments, the disease, disorder or condition is ulcerative colitis.

In some embodiments, the disclosure provides a method for treating or preventing a disease, disorder or condition associated with type 2 diabetes or predisposition for diabetes in a subject needing such treatment or prevention, the method comprising administering an effective amount of a pharmaceutical composition provided herein (e.g., any one of [66]-[68] in the Brief Summary) to the subject. In some embodiments, the disease, disorder or condition is metabolic disease. In some embodiments, the disease, disorder or condition is insulin resistance. In some embodiments, the disease, disorder or condition is a diabetic vascular disease (e.g., a microvascular disease such as retinopathy and nephropathy). In some embodiments, the disease, disorder or condition is diabetic neuropathy. In some embodiments, the disease, disorder or condition is ulcers, diabetic necrosis, or gangrene.

In some embodiments, the disclosure provides a method for treating or preventing a myopathy, chronic microvascular disease, or microangiopathy, or a disorder associated with microvascular dysfunction such as age-related macular degeneration (AMD) in a subject needing such treatment or prevention, the method comprising administering an effective amount of a pharmaceutical composition provided herein (e.g., any one of [66]-[68] in the Brief Summary) to the subject.

In some embodiments, the disclosure provides a method for treating or preventing a disease, disorder or condition associated with sclerosis in a subject needing such treatment or prevention, the method comprising administering an effective amount of a pharmaceutical composition provided herein (e.g., any one of [66]-[68] in the Brief Summary) to the subject. In some embodiments, the disease, disorder or condition associated with sclerosis is systemic sclerosis.

In some embodiments, the disclosure provides a method for treating endotoxemia in a subject needing such treatment, the method comprising administering an effective amount of a pharmaceutical composition provided herein (e.g., any one of [66]-[68] in the Brief Summary) to the subject. In some embodiments, the endotoxemia is associated with a condition such as periodontal disease (e.g., periodontitis or inflammation of the gums), chronic alcoholism, chronic smoking, transplantation, or neonatal necrotizing enterocolitis, or neonatal ear infection.

In some embodiments, the disclosure provides a method of reducing systemic levels of LPS, endotoxin and/or another trigger of systemic inflammation in a subject in need thereof, the method comprising administering an effective amount of a pharmaceutical composition provided herein (e.g., any one of [66]-[68] in the Brief Summary) to the subject.

### Combination Therapy

The compositions provided herein can be administered alone or in combination therapy with one or more additional therapeutic agents. In some embodiments, the composition is administered in combination therapy with another therapeutic agent. Combinations may be administered either concomitantly, e.g., combined in the same liposomal composition, delivery vehicle (e.g., liposome), as an admixture, separately but simultaneously or concurrently; or sequentially. This includes presentations in which the combined therapeutic agents are administered together as a therapeutic mixture, and also procedures in which the combined agents are administered separately but simultaneously, e.g., as through separate intravenous lines into the same individual. Administration “in combination” further includes the separate administration of one of the therapeutic agents given first, followed by the second. Methods of treatment using the combination therapy are also provided.

In additional embodiments, a composition provided herein is administered in combination with another therapeutic agent. In some embodiments, a pharmaceutical composition comprising any of the carotenoid of Formula I (e.g., Formula I-1, I-2, such as those described in [54]-[56] in the Brief Summary or any of the substantially pure TSC described herein) is administered in combination with another therapeutic agent. In some embodiments, an aqueous solution comprising a carotenoid of Formula I-1 (e.g., any of the substantially pure TSC described herein) is administered in combination therapy with another therapeutic agent. In some embodiments, a composition comprising a multivalent salt (e.g., a monovalent salt or a trivalent salt) of a carotenoid of Formula I (e.g., Formula I-1, I-2, such as those described in [54]-[56] in the Brief Summary or any of the substantially pure TSC described herein) is administered in combination therapy with another therapeutic agent. In particular embodiments, a composition comprising a multivalent salt of trans-crocetin (e.g., CTC or MTC) is administered in combination therapy with another therapeutic agent. In other particular embodiments, a composition comprising a multivalent salt of trans-norbixin (e.g., CTN or MTN) is administered in combination therapy with another therapeutic agent.

In some embodiments, a pharmaceutical composition comprising a salt of one or more ionizable carotenoids is administered in combination therapy with a carotenoid comprising at least one polar group or monocyclic group. In some embodiments, the salt of the ionizable carotenoid is a multivalent salt (e.g., salt containing monovalent, trivalent or tetravalent counterion). In some embodiments the ionizable carotenoid is a carotenoid of Formula I (e.g., Formula I-1, I-2, such as those described in [54]-[56] in the Brief Summary or any of the substantially pure TSC described herein). In one embodiment, the carotenoid comprising at least one polar group or monocyclic group polar group is symmetric. In another embodiment, a monovalent ionizable carotenoid salt composition is administered in combination therapy with at least one carotenoid selected from: zeanthin, astaxanthin, lutein, and xanthophyll. In another embodiment, the monovalent ionizable carotenoid salt composition is administered in combination therapy with astaxanthin. In another embodiment, the carotenoid comprising at least one polar group or monocyclic group polar group is asymmetric. In another embodiment, a monovalent ionizable carotenoid salt composition disclosed herein is administered in combination abscisic acid (ABA).

In some embodiments, a pharmaceutical composition comprising an ionizable carotenoid salt provided herein is administered in combination therapy with a standard of care treatment for the disease, disorder, or condition to be treated. In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an antimicrobial agent. In some embodiments, the antimicrobial agent is an anti-bacterial agent. In some embodiments, the antibacterial agent is selected from, but not limited to, ertapenem, piperacillin-tazobactam, cefepime, aztreonam, metronidazole, meropenem, ceftriaxone, ciprofloxacin, vancomycin, linezolid, tobramycin, levofloxacin, azithromycin, cefazolin, and ampicillin. In some embodiments, the antibacterial agent is selected from, but not limited to, ceftriaxone, levofloxacin, ciprofloxacin, cefazolin, piperacillin-tazobactam, meropenem, metronidazole, vancomycin, and ampicillin. In other embodiments, the antimicrobial agent is an anti-fungal agent. In further embodiments, the anti-fungal agent is caspofungin or another antifungal drug. In other embodiments, the antimicrobial agent is an anti-malarial agent. In further embodiments, the anti-malarial agent is selected from, but not limited to, artemisinin and its analogs, chloroquin and its analogs, atovaquone, a quinine derivative, proguanil or another anti-malarial drug. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with activated protein C (e.g., rhAPC), or drotrecogin alfa (activated) (DAA). In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a corticosteroid (e.g., a glucocorticoid or mineralocorticoid such as fludrocortisonel). In some embodiments, the corticosteroid is a glucocorticoid. In further embodiments, the glucocorticoid is selected from cortisone, ethamethasoneb, prednisone, prednisolone, triamcinolone, dexamethasone and methylprednisolone. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with intravenous administration of a vitamin. In some embodiments, the vitamin is vitamin C (ascorbic acid). In some embodiments, the vitamin is vitamin A. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a glucocorticoid and vitamin C (e.g., intravenous vitamin C administration). In some embodiments, the glucocorticoid is selected from cortisone, ethamethasoneb, prednisone, prednisolone, triamcinolone, dexamethasone and methylprednisolone. In further embodiments, the glucocorticoid is hydrocortisone. In additional embodiments, at least one ionizable carotenoid composition provided herein (e.g., a monovalent or bivalent salt composition comprising an ionizable carotenoid carotenoid of Formula I (e.g., Formula I-1, I-2, such as those described in [54]-[56] in the Brief Summary or any of the substantially pure TSC described herein)) is administered in combination therapy with a glucocorticoid, vitamin C, and thiamine. In some embodiments, the salt of the ionizable carotenoid is a monovalent salt. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a vasopressor agent. In some embodiments, the vasopressor therapeutic agent is norepinephrine or similar drugs, or angiotensin II (e.g., GIAPREZA™) In some embodiments, the vasopressor therapeutic agent is epinephrine, phenylnephrine, dopamine, or vasopressin. In some embodiments, the vasopressor therapeutic agent is ephedrine, milrinone, isoproterenol, dobutamine, isoproterenol, or dopamine.

In some embodiments, a pharmaceutical composition comprising an ionizable carotenoid salt provided herein is administered in combination therapy with a thrombolytic therapeutic agent. In some embodiments, the thrombolytic therapeutic agent tissue plasminogen activator (tPA). In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In additional embodiments, a pharmaceutical composition comprising an ionizable carotenoid salt provided herein is administered in combination therapy with an anesthetic agent. In some embodiments, the anesthetic agent is administered before the pharmaceutical composition (e.g., as an anesthetic preconditioning (APC) regimen, prior to surgery). In some embodiments, the anesthetic agent is administered after the pharmaceutical composition (e.g., post-surgery). In some embodiments, anesthetic agent is isoflurane, sevoflurane, or propofol. In some embodiments, anesthetic agent is cystathionine-β-synthase (CBS), cystathionine-γ-lyase (CSE), or 3-mercapto-pyruvate-sulfur-transferase (MST). In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a therapeutic agent. In some embodiments, a pharmaceutical composition of any of [66]-[68] is administered in combination with a therapeutic agent. In some embodiments, a pharmaceutical composition comprising a multivalent salt of a carotenoid of Formula I herein, is administered in combination therapy with a therapeutic agent. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a therapeutic agent selected from: heparin, vasopressin, antidiuretic hormone (ADH), and a 3-Hydroxy-3-methylglutaryl coenzyme A reductase inhibitor (statin). In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an anti-inflammatory therapeutic agent. In some embodiments, the salt of the ionizable carotenoid is a multivalent salt (e.g., bivalent, trivalent or tetravalent). In some embodiments the ionizable carotenoid is a carotenoid carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with oxygen and/or intravenous fluids to maintain/increase blood oxygen levels and/or blood pressure or hyperbaric therapy. In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an antioxidant. In some embodiments, the ionizable carotenoid salt provided herein is administered in combination therapy with at least one of alpha-tocopherol, melatonin, ascorbic acid (AA), alpha lipoic acid, desferoxamine, and trimetazidine (TMZ). In some embodiments, the ionizable carotenoid salt provided herein is administered in combination therapy with at least one of glutatione, N-Acetylcysteine (NAC), Bucillamine (N-(2-mercapto-2-methylpropionyl)-1-cysteine), a superoxide dismutase (SOD) or derivative thereof, catalase (CAT), and allopurinol, idebenone. In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the administered ionizable carotenoid is trans-crocetin (e.g., CTC or STC). In other particular embodiments, the administered ionizable carotenoid is trans-norbixin (e.g., CTN and STN).

In some embodiments, a pharmaceutical composition comprising an ionizable carotenoid salt provided herein is administered in combination therapy with a chemotherapeutic agent (e.g., to enhance the effect of chemotherapy on cancer cells and mitigate the effects of chemotherapy-induced myelosuppression and anemia). In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with immunotherapy. In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with radiotherapy. In some embodiments, the salt of the ionizable carotenoid is a multivalent salt (e.g., bivalent, trivalent or tetravalent). In some embodiments the ionizable carotenoid is a carotenoid of Formula I (e.g., Formula I-1, I-2, such as those described in [54]-[56] in the Brief Summary or any of the substantially pure TSC described herein). In particular embodiments, the ionizable carotenoid is trans-crocetin (e.g., CTC or STC). In other particular embodiments, the ionizable carotenoid is trans-norbixin (e.g., CTN and STN).

### Kits for Administration of Active Agents

In another embodiments, the disclosure provides a kit for administering a pharmaceutical composition provided herein (e.g., any one of [66]-[68] in the Brief Summary) to a subject for treating a disease, disorder, or condition. In some embodiments, the disclosure provides a kit for delivering a therapeutic agent to a subject, the kit comprising: (a) a first composition comprising a disclosed liposome comprising a carotenoid of Formula I (e.g., Formula I-1, I-2, such as those described in [54]-[56] in the Brief Summary or any of the substantially pure TSC described herein) and (b) a second composition containing for example, reagents, buffers, excipients, or another therapeutic agent that is stored separately prior to administration to the subject. Such kits typically include two or more components necessary for treating a disease state, such as hypoxia or inflammation related condition. In some embodiments, the kits include for example, a provided aqueous solution, compositions, reagents, buffers, containers and/or equipment. In some embodiments, the kits include a packaging assembly that include one or more components used for treating the disease state of a patient. For example, a packaging assembly may include separate containers that house the reagents necessary to formulate the aqueous solutions and other excipients or therapeutic agents that can be mixed with the compositions prior to administration to a patient. In some embodiments, a physician may select and match certain components and/or packaging assemblies depending on the treatment or diagnosis needed for a particular patient.

### EXAMPLES

### Example 1—Synthesis of Trans Sodium Crocetinate

The following presents exemplary process for preparing TSC. The overall scheme is shown below:

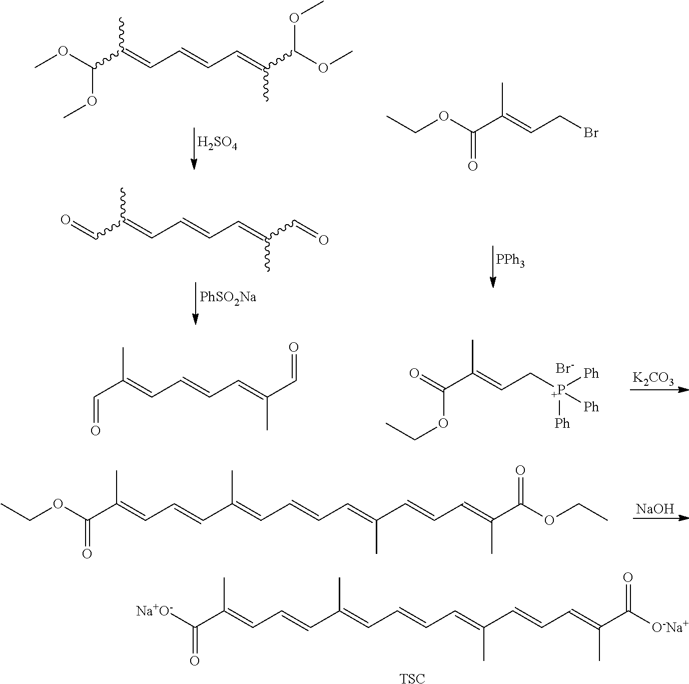

### Step 1: Synthesis of C10-Cis/Trans Dial

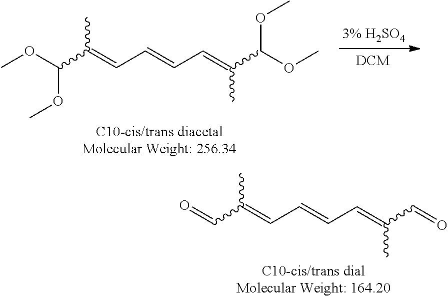

6.8 kg of C10-Cis/Trans Diacetal was added into 10.5 L dichloromethane (DCM), followed by slow addition of 6.8 kg of 3% H₂SO₄ over an hour. The reaction mixture was stirred at room temperature for 3 hrs. The reaction completion was monitored by GC (IPC-1, C10-Cis/Trans Dial by GC) to confirm the reaction completion.

After reaction was complete, 5.0 L DCM was added to the reaction mixture to dissolve any solid on the reactor wall, (IPC-2, solid on the side wall dissolved), organic DCM layer was collected and aqueous layer was then extracted with DCM, which was combined with the collected DCM phase. This organic phase was washed with saturated aqueous sodium bicarbonate to pH≥7 (IPC-3, pH≥7), collected into a pail, and dried over anhydrous Na₂SO₄. The aqueous layer was extracted with n-heptane, and after confirming that both aqueous and organic layers were clear (IPC-4, both organic and aqueous layers are clear), the organic layer was collected in a pail and dried over Na₂SO₄.

The dried DCM and n-heptane solution were then mixed with silica gel, and the solvents were evaporated in a Rotavap to give a solid mixture. The half of this solid mixture was loaded onto silica gel in a 5 Gal chromatography column, eluted with 15 L n-heptane to remove mineral oil. A fraction of collected n-heptane eluent was concentrated down by rotary evaporation to confirm the mineral oil was removed (IPC-5, completion of n-Heptane elution to remove mineral oil). The column was then eluted with 30 L DCM, and eluent was concentrated by rotary evaporation to obtain the first half of the product. The elution end point was confirmed by collecting an eluent fraction and dried using a Rotavap to confirm most of the product was collected (IPC-6, completion of DCM elution). After DCM was removed by rotary evaporation, product of C10-Cis/Trans Dial was transferred to glass trays and dried in a vacuum dryer at room temperature.

The second half of the solid mixture was purified using the same method described above (IPC-7, completion of n-Heptane elution to remove mineral oil; IPC-8, completion of DCM elution). A total of 3.2 kg product was produced from 6.8 kg of C10-Cis/Trans Diacetal, yield 74% (Table 1). A representative HPLC trace of the cis/trans dial is shown in FIG. 1, wherein the desired trans dial is present in an amount of about 63.22% by HPLC area percentage (retention time 15.22), and the cis dial in a total amount of about 25% by HPLC area percentage (retention times 16.22 and 16.61).

The HPLC conditions used for this analysis are the following:

### Instrument

Agilent Separations Module, which includes degasser, pump, autosampler and UV detector,

### Mobile Phase

Eluent A: Dissolve 3.40 g of KH₂PO₄ (25 mmol) with 1000 mL of HPLC grade water. The pH is adjusted to 5.0±0.1 with 14% KOH using a calibrated pH meter. The solution is filtered through at least a 0.45 μm filter and degassed prior to use.
Eluent B: Acetonitrile (ACN),
Column: Zorbax Eclipse plus C18, 250×4.6 mm, 5 μm,
   Agilent, or equivalent,
Flow rate: 1.0 mL/min
Injection volume: 5 μL
Wavelength: 330 nm
Column oven temperature: 35° C.
Sample compartment temperature: room temperature
Sample run time: 25 minutes
Total run time: 33 minutes

### Gradient Conditions:

| Time (minutes) | Eluent A (%) | Eluent B (%) | Flow (mL/min) |
|---|---|---|---|
| 0 | 70 | 30 | 1.00 |
| 5 | 70 | 30 | 1.00 |
| 15 | 60 | 40 | 1.00 |
| 20 | 20 | 80 | 1.00 |
| 25 | 20 | 80 | 1.00 |
| 25.1 | 70 | 30 | 1.00 |
| 33 | 70 | 30 | 1.00 |

TABLE 1
Batch Summary of C10-Cis/Trans Dial

| | Quantity | HPLC Purity | Yield % |
|---|---|---|---|
| C10-Cis/Trans dial | 3.2 kg | 98.6% (1^{st} half) | 74% |
| | | 98.5% (2^{nd} half) | |

### Step 2: Synthesis of C10-Trans Dial

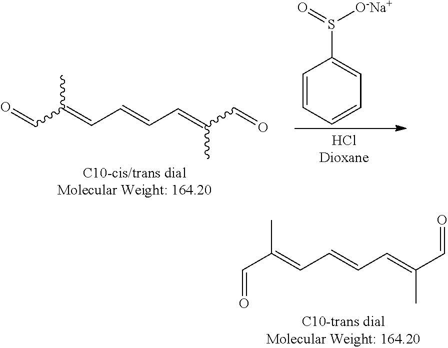

3.2 kg of C10-Cis/Trans Dial was added into a mixture of 390 mL 4M HCl in 1,4-dioxane and 320 g sodium benzenesulfinate in 15.5 L 1,4-dioxane, stirred and heated to 75° C., and stirred for 9 hrs. The reaction completion was monitored by HPLC (IPC-1, Trans C10-dial ratio doesn't increase overtime). The reaction mixture was then cooled to room temperature, concentrated by rotary evaporation to obtain a solid. This solid was then dissolved using 26 L of DCM, washed with distilled water and with saturated aqueous sodium bicarbonate. The organic and aqueous layers were separated. After confirming aqueous layer pH was ≥7, (IPC-2, pH test), the organic layer was washed with distilled water. The pH of separated aqueous layer was checked again to confirm pH≥7 (IPC-3, pH test). Then the organic layer was dried over anhydrous Na₂SO₄, filtered, and concentrated by rotary evaporation to obtain a yellow solid product. This yellow solid was added to 2 L 1:1 DCM/n-heptane to make a slurry, filtered with a ceramic filter. The filtered cake was washed with n-heptane and methyl tert-butyl ether (MTBE), dried in a vacuum dryer at room temperature to obtain 2.8 kg of C10-Trans Dial crude.

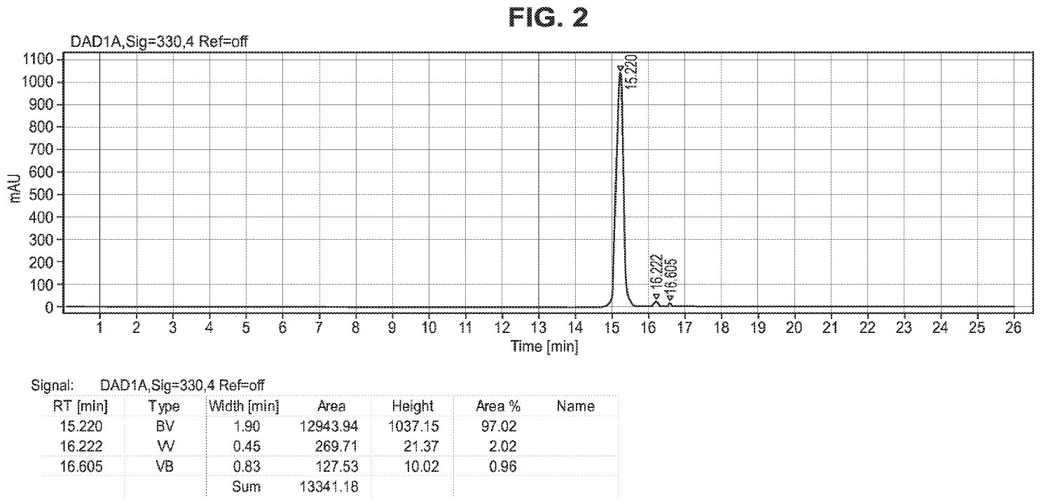

2.8 kg of C10-Trans Dial crude was added into 8.4 L THF, heated to reflux for 1 hr, cooled to room temperature over 1 hr then further cooled with ice bath. The product was then collected by filtration and washed with THF, dried in vacuum dryer at room temperature to give the pure C10-Trans Dial, 2.2 kg, yield 69% (Table 2). A representative HPLC trace of the C10-Trans Dial is shown in FIG. 2, wherein the desired trans dial is present in an amount of about 97% by HPLC area percentage (retention time 15.22), and the cis dials in a total amount of less than 3% by HPLC area percentage (retention times 16.22 and 16.61). The HPLC conditions used for this analysis are the same as shown in step 1 above.

TABLE 2
Batch Summary of C10-Trans Dial

| | Quantity | HPLC Purity | Yield % |
|---|---|---|---|
| Trans C10-dial | 2.2 kg | 97.0% | 69% |

### Step 3: Synthesis of C20-Trans Diester

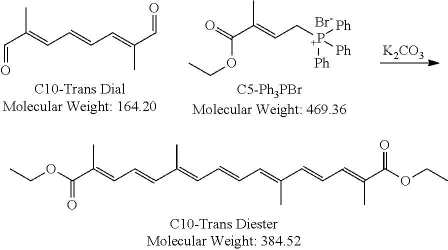

In 12.5 L of toluene, 8.6 kg C5-Ph₃PBr (prepared from reacting PPh₃ with the corresponding bromide) and 1.25 kg of Trans C10-dial were added, followed by 3.2 kg of K₂CO₃ and 3.8 L of MeCN. The reaction mixture was heated to 58° C. for 4 hrs. The reaction progress was monitored by HPLC (IPC-1, reaction intermediates % by HPLC). After the intermediates was confirmed ≤4%, the reaction mixture was heated to 80° C. for 20 hours, cooled to room temperature and further cooled to 5° C. The reaction mixture was filtered, and the filtered cake was washed with MeCN, followed by distilled water to remove residual K₂CO₃ (IPC-2, pH of wash=7).

The collected solid was slurried with 4 L methanol twice to remove residual triphenyl phosphine oxide (IPC-3, residual triphenyl phosphine oxide by HPLC≤0.2%), dried in a vacuum dryer, and packed into two portions in PE bags.

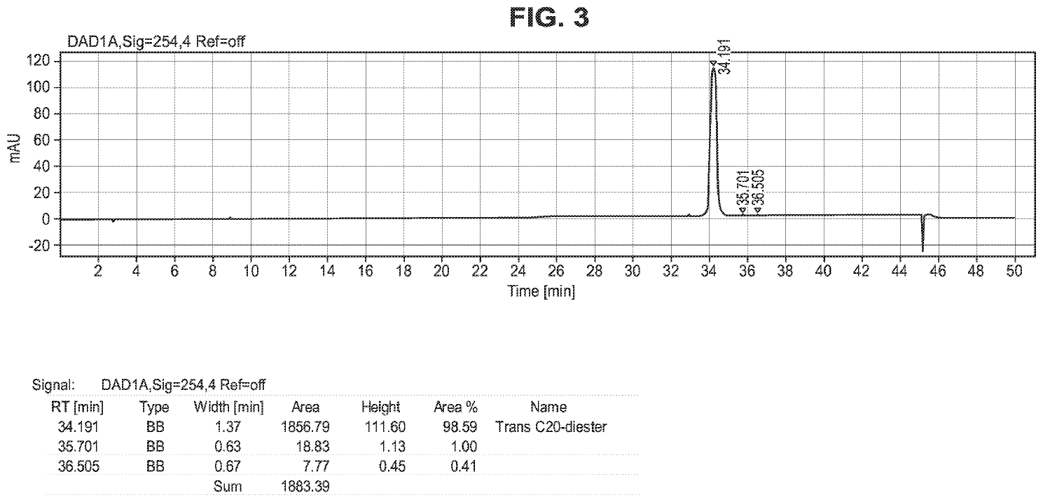

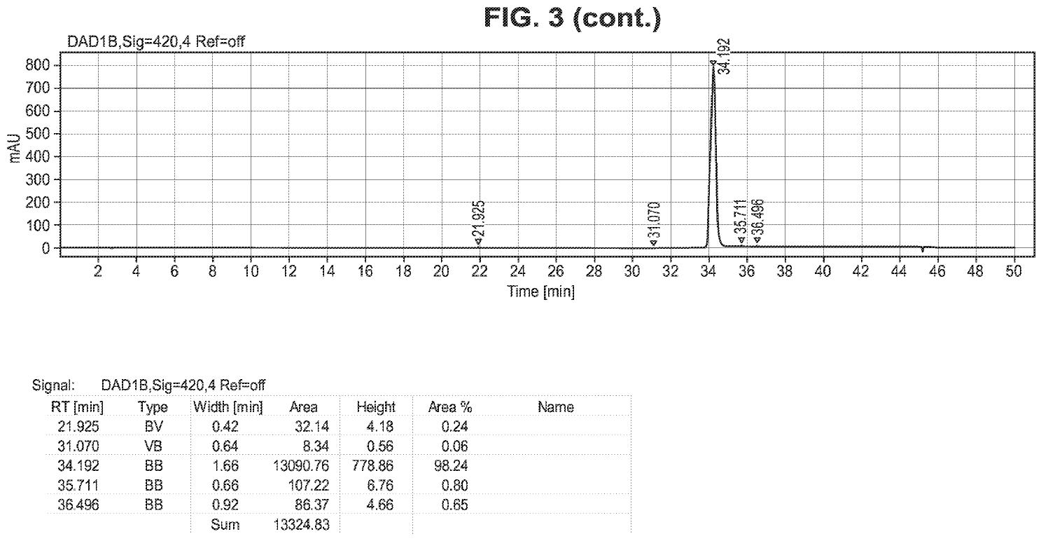

Each portion of the product was then dissolved with DCM (IPC-4, DCM is a clear solution of Trans C20-Diester, and IPC-6, DCM is a clear solution of Trans C20-Diester), washed with distilled water. The organic and aqueous layers were separated. The pH of the aqueous layer was checked with a pH paper (IPC-5, pH test, aqueous layer pH=7 and IPC-7, pH test, aqueous layer pH=7). The organic layer was dried over anhydrous Na₂SO₄, filtered, and concentrated by rotary evaporation to give a solid. This solid was slurried with ethanol, filtered, and dried in a vacuum dryer at room temperature to give the final product of C20-Trans Diester, 1.43 kg, yield 49% (Table 3). A representative HPLC trace of the C20-Trans Diester is shown in FIG. 3, wherein the desired trans diester is present in an amount of about 98% by HPLC area percentage (retention time 34.19) at either detection wavelength of 254 or 420 nm.

The HPLC conditions used for this step are the following:

### Instrument

Agilent Separations Module, which includes degasser, pump, autosampler and UV-Vis detector.

### Mobile Phase

Eluent A: Aliquot 1 mL phosphoric acid solution into 1000 mL of HPLC grade water to obtain 0.1% v/v H₃PO₄ aqueous solution,
Eluent B: Acetonitrile.
Column: Zorbax Eclipse Plus C18, 250×4.6 mm, 5 μm, Agilent, or equivalent.
Flow rate: 1.0 mL/min
Injection volume: 5 μl
Wavelength: 254 nm and 330 nm for IPC test
   254 nm and 420 nm for Trans C20-diester purity test
Column oven temperature: 35° C.
Sample compartment temperature: room temperature
Sample run time: 42 minutes
Total run time: 50 minutes

### Gradient Conditions:

| Time (minutes) | Eluent A (%) | Eluent B (%) | Flow (mL/min) |
|---|---|---|---|
| 0 | 70 | 30 | 1.00 |
| 20 | 20 | 80 | 1.00 |
| 23 | 15 | 85 | 1.00 |
| 42 | 15 | 85 | 1.00 |
| 42.1 | 70 | 30 | 1.00 |
| 50 | 70 | 30 | 1.00 |

TABLE 3
Batch Summary of C20-Trans Diester

| | Quantity | HPLC Purity | Yield % |
|---|---|---|---|
| Trans C20-Diester | 1.43 kg | 98.2% | 49% |

### Step 4: Synthesis of Trans Sodium Crocetinate (TSC)

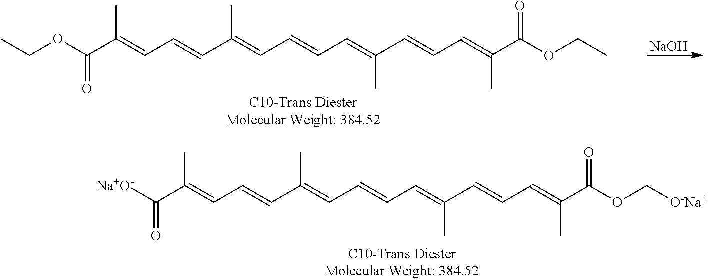

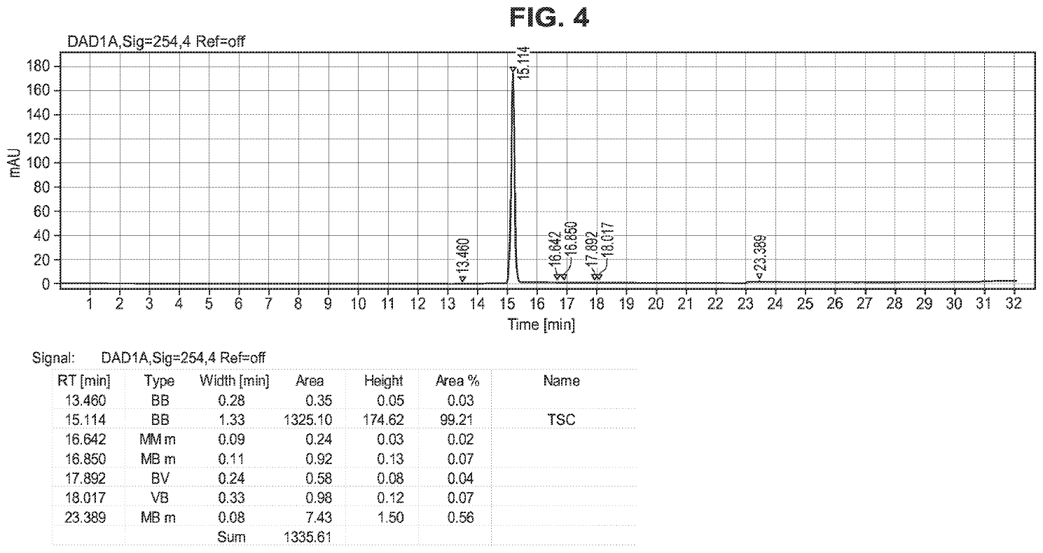

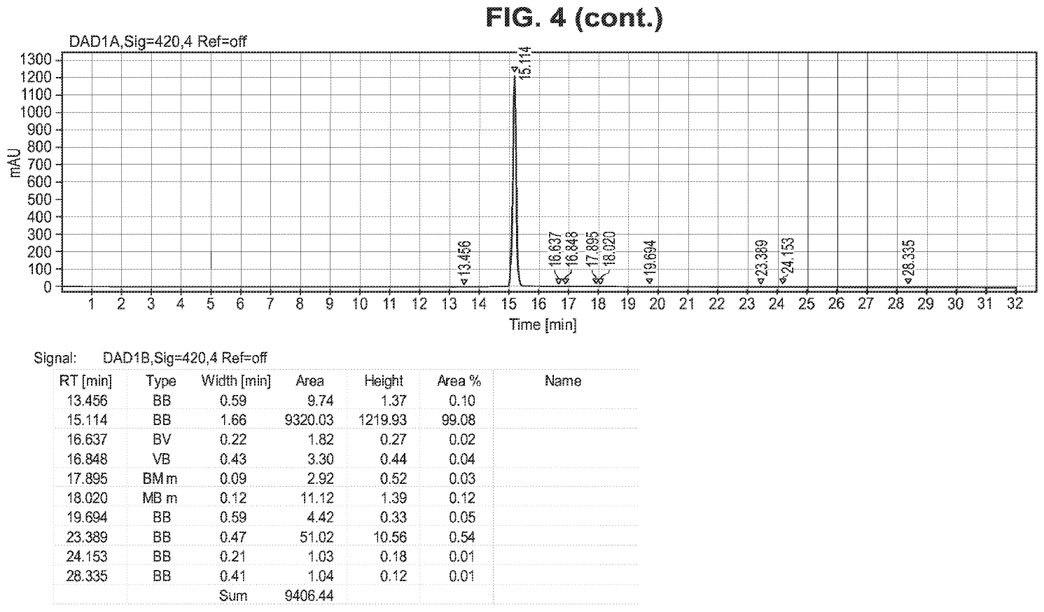

1.35 kg of Trans C20-Trans Diester was added into 5.4 kg of 15% NaOH solution, followed by 6.8 L pure ethanol. The reaction mixture was heated to reflux for 5.5 hrs. HPLC analysis was used (IPC-1, Trans C20-Diester % by HPLC) to monitor the reaction progress. After reaction was completed, the reaction was cooled to room temperature, filtered with a ceramic filter to collect the product. Excess NaOH in the reaction mixture was washed off with distilled water, which was monitored by checking the pH value of the filtrate (IPC-2, pH of wash by pH meter, pH=10.0±0.3). Then the filtered cake was washed with pure ethanol, dried in a vacuum dryer at 30° C. to give the final product Trans Sodium Crocetinate (TSC) as yellow solid, 984 g, yield 76% (Table 4). A representative HPLC trace of TSC is shown in FIG. 4, wherein the TSC is present in an amount of about 98% by HPLC area percentage (retention time 15.11) at either detection wavelength of 254 or 420 nm.

HPLC conditions used for the analysis of purity of TSC:

### Instrument

Agilent Separations Module, which includes degasser, pump, autosampler and UV-Vis detector.

### Mobile Phase

Eluent A: Aliquot 1 mL phosphoric acid solution into 1000 mL of HPLC grade water to obtain 0.1% v/v H₃PO₄ aqueous solution.
Eluent B: Acetonitrile.
Eluent C: Methanol,
Column: Zorbax Eclipse Plus C18, 250×4.6 mm, 5 μm,
   Agilent, or equivalent.
Flow rate: 1.0 mL/min
Injection volume: 5 μL
Wavelength: Purity: 254 nm and 420 nm
Assay: 420 nm
Column oven temperature: 35° C.
Sample compartment temperature: 5° C.
Sample run time: 39 minutes
Total run time: 47 minutes

Gradient Conditions:

| Time | Eluent A | Eluent B | Eluent C | Flow |
|---|---|---|---|---|
| (minutes) | (%) | (%) | (%) | (mL/min) |
| 0 | 70 | 30 | 0 | 1.00 |
| 4 | 60 | 40 | 0 | 1.00 |
| 12 | 52 | 48 | 0 | 1.00 |
| 14 | 52 | 48 | 0 | 1.00 |
| 23 | 38 | 62 | 0 | 1.00 |
| 28 | 38 | 62 | 0 | 1.00 |
| 32 | 0 | 85 | 15 | 1.00 |
| 39 | 0 | 85 | 15 | 1.00 |
| 39.1 | 70 | 30 | 0 | 1.00 |
| 47 | 70 | 30 | 0 | 1.00 |

TABLE 4
Batch Summary of Trans Sodium Crocetinate (TSC)

| | HPLC Purity | Yield % |
|---|---|---|
| Trans Sodium | 99.2% at 254 nm | 76% |
| Crocetinate (TSC) | 99.1% at 420 nm | |

Further, none of the impurities peaks for triphenylphosphine oxide, the trans C20 diester, trans C10-dial, and the C5-PPh₃PBr appeared in the HPLC trace for the TSC obtained in this example. All single unknown impurities in the HPLC trace was less than 0.8% and the total impurity is less than 2%.

The TSC obtained was also assayed using a reference standard by HPLC, see details below. The HPLC conditions were shown above.

Calculate the weight/weight assay content of TSC in each sample (based on anhydrous basis) as follows:

$$\% TSC = \frac{{A}_{S} \times {W}_{R} \times {P}_{R} \times ( 1 - {H}_{2} O {\%}_{R} )}{{A}_{R} \times {W}_{S} \times ( 1 - {H}_{2} O {\%}_{\%} )} \times 100 \%$$

### Where

A_{S}: peak area of Sample Preparation
A_{R}: average peak area of the first 5 injections of Standard Preparation 1
W_{S}: weight (mg) of Sample Preparation
W_{R}: weight (mg) of Standard Preparation 1

### P_{R}: HPLC Purity of TSC Reference Standard

H₂O %_{S}: water content of Sample
H₂O %_{R}: water content of Reference Standard
The assay of TSC at is reported using 420 nm chromatogram data.

Based on this study, the sample prepared following this procedure was found to be 99.3% pure by weight, on anhydrous basis. The water content was 2.8% of the TSC sample prepared in this example, using the Karl Fischer method.

Further, the sodium content of the TSC sample prepared in this example was also determined using Inductively Coupled Plasma Optical Emission Spectrometry (ICP-OES). The sodium content was determined to be 11.7%, on anhydrous basis. The procedures for using ICP-OES are known. Typically, it involves calibrate the ICP instrument using the Calibration Standard(s) Preparation. Sequentially aspirate each Sample Preparation, and the Reagent Spike (if necessary) into the ICP instrument. Calculate the content for each element in the Sample Preparation as follows:

$$( E ) \% = \frac{response ( E ) \times dilution factor \times {ppmStd}_{E} \times 100}{response ( {Std}_{E} ) \times sample weight ( μ g )}$$

### Where:

(E) %=percent of the individual element in the Sample Preparation
response(E)=optical emission of the individual element in the Sample Preparation
dilution factor=final diluted volume of the sample, blank and standards preparations
ppmStd_{E}=ppm of the respective element added to the Reagent Spike Preparation, if required
100=conversion to percent
response(Std_{E})=optical emission of the individual element in the Reagent Spike Preparation, if necessary
Sample weight (μg)=weight of the sample, in micrograms, in the Sample Preparation.

Elemental impurities, microbials, bacterial endotoxins were also tested for the TSC sample prepared in this example following USP<232>, USP<233>, USP<61>, USP<62>, USP<85> respectively. For example, the levels of Cd, Pb, As, Hg, Co, V, Ni, Li, Sb, and Cu were all tested to be within specification (the left column is the specification and the right column shows the test results):

| | | | | |
|---|---|---|---|---|
| Cadmium (Cd) | ≤0.2 | ppm | <0.10 | ppm |
| Lead (Pb) | ≤0.5 | ppm | 0.17 | ppm |
| Arsenic (As) | ≤1.5 | ppm | <0.10 | ppm |
| Mercury (Hg) | ≤0.3 | ppm | <0.10 | ppm |
| Cobalt (Co) | ≤0.5 | ppm | <0.10 | ppm |
| Vanadium (V) | ≤1 | ppm | <0.10 | ppm |
| Nickel (Ni) | ≤2 | ppm | 1.23 | ppm |
| Lithium (Li) | ≤25 | ppm | <0.10 | ppm |
| Antimony (Sb) | ≤9 | ppm | <0.10 | ppm |
| Copper (Cu) | ≤30 | ppm | <0.10 | ppm |

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

The disclosure of each of U.S. Appl. No. 63/007,878, filed Apr. 9, 2020 and Intl. Publ. No. WO2019213538, is herein incorporated by reference in its entirety.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

What is claimed is:

1. A method of preparing a carotenoid having Formula I:
Q-Y-Polyene Carotenoid-Y-Q, (I)
wherein,
the Polyene Carotenoid comprises
(a) 3, 4, 5, 6, 7, 8, 9, 10, 3-5, 6-8, 9-10, or more than 9, conjugated double bonds, and
(b) methyl or low alkyl (C2-C3) substitutions, each Y is independently a bond, 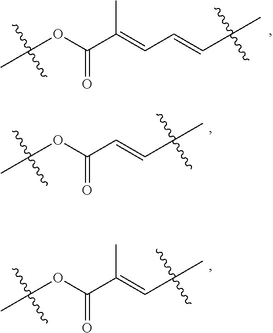
or a geometric isomer thereof, provided that at least one Y has a —C(O)—O— functional group, and
each Q is independently an ionizable group, hydrogen, an optionally substituted C₁₋₄ alkyl, or a counterion,
the method comprising:
a) reacting an aldehyde of Formula II with an olefin forming agent to form an ester (e.g., a C₁₋₄ alkyl ester, such as ethyl ester):
Q²-Y²-Polyene Carotenoid-Y²-Q², (II)
wherein at least one of Q²-Y² is HC(O)—, and when only one of Q²-Y² is HC(O)—, the other Q²-Y² is the same as Y-Q- in Formula I,
wherein the olefin forming agent is capable of reacting with the aldehyde to form a structural moiety of 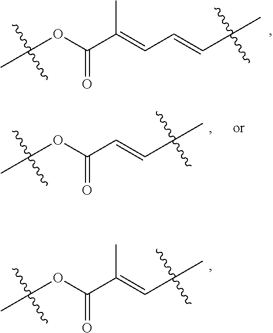
or a geometric isomer thereof,
and
b) optionally, when at least one Q is hydrogen or a counterion, hydrolyzing the ester to form the carotenoid of Formula I.

2. The method of claim 1, wherein both Y are independently selected from 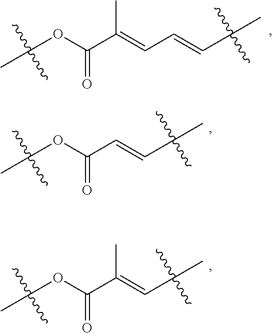
or a geometric isomer thereof.

3. The method of claim 1 or 2, wherein both Y are the same.

4. The method of any one of claims 1-3, wherein both Q are independently selected from hydrogen, an optionally substituted C₁₋₄ alkyl, or a counterion.

5. The method of any one of claims 1-4, wherein both Q are the same.

6. The method of claim 5, wherein both Q are C₁₋₄ alkyl.

7. The method of claim 5, wherein both Q are hydrogen.

8. The method of claim 5, wherein both Q are monovalent counterion, e.g., alkali metal ion, such as Li⁺, Na⁺, or K⁺.

9. The method of any one of claims 1-8, wherein the Polyene Carotenoid is a hydrocarbon group having a total number of 6-36 carbons, such as 8, 10, 12, 14, 16, 18, or 20 carbons.

10. The method of any one of claims 1-9, wherein the olefin forming agent is a phosphine based olefin forming agent.

11. The method of any one of claims 1-10, wherein the olefin forming agent has the Formula III-1, 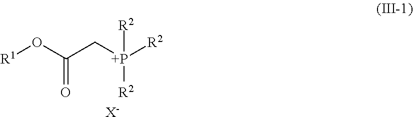
wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide.

12. The method of any one of claims 1-11, wherein the olefin forming agent has the Formula of III-1-A: 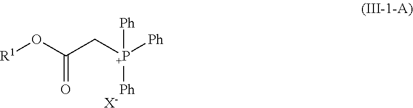
wherein R¹ is an optionally substituted C₁₋₄ alkyl, and X⁻ is a counterion, e.g., a halide such as chloride or bromide.

13. The method of any one of claims 1-10, wherein the olefin forming agent has the Formula III-2, 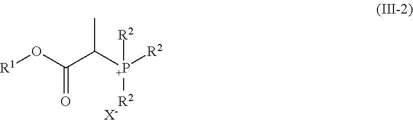
wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide.

14. The method of any one of claims 1-10 and 13, wherein the olefin forming agent has the Formula of III-2-A: 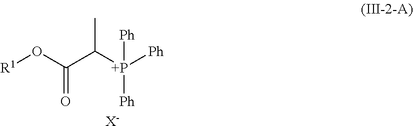
wherein R¹ is an optionally substituted C₁₋₄ alkyl, and X⁻ is a counterion, e.g., a halide such as chloride or bromide.

15. The method of any one of claims 1-10, wherein the olefin forming agent has the Formula III-3, 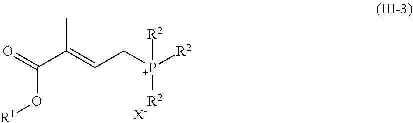
wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide.

16. The method of any one of claims 1-10 and 15, wherein the olefin forming agent has the Formula III-3-A: 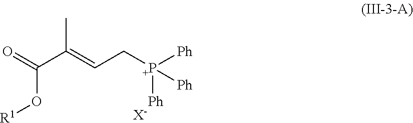
wherein R¹ is an optionally substituted C₁₋₄ alkyl, and X⁻ is a counterion, e.g., a halide such as chloride or bromide.

17. The method of any one of claims 1-16, wherein R¹ is not methyl.

18. The method of any one of claims 1-17, wherein R¹ is ethyl.

19. The method of any one of claims 1-18, wherein the olefin forming reaction is carried out in a non-ether solvent in the presence of a base such as an inorganic base.

20. The method of any one of claims 1-19, wherein the olefin forming reaction is carried out in a solvent system comprising acetonitrile, e.g., comprising acetonitrile and toluene.

21. The method of claim 19 or 20, wherein the inorganic base is a carbonate base, e.g., sodium carbonate, potassium carbonate, sodium bicarbonate, potassium bicarbonate, etc.

22. The method of any one of claims 1-21, wherein the carotenoid of formula I has the Formula I-1: 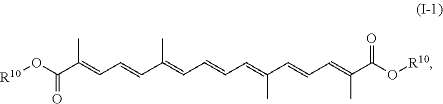
or a geometric isomer thereof, or a combination thereof,
wherein each R¹⁰ is independently selected from hydrogen, a counterion (e.g., a monovalent counterion or a divalent counterion), and an optionally substituted C₁₋₄ alkyl, preferably, both R¹⁰ are the same.

23. The method of claim 22, wherein both R¹⁰ are C₁₋₄ alkyl, preferably, ethyl.

24. The method of claim 23, wherein both R¹⁰ are a monovalent counterion, preferably, an alkali metal ion, such as Li⁺, Na⁺, or K⁺.

25. The method of claim 24, wherein both R¹⁰ are hydrogen.

26. The method of any one of claims 22-25, wherein the aldehyde of formula II has the Formula II-1: 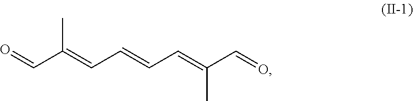
or a geometric isomer thereof, or a combination thereof.

27. The method of claim 26, wherein the aldehyde of formula II used for reacting with the olefin forming agent is a substantially pure form of Formula II-1: 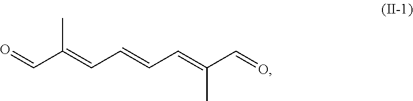
which includes less than 10% of other geometric isomers by HPLC area percentage with detection wavelength at 330 nm, preferably, less than 5% such as less than 3%, or less than 2% of other geometric isomers.

28. The method of any one of claims 1-21, wherein the carotenoid of formula I has the Formula I-2: 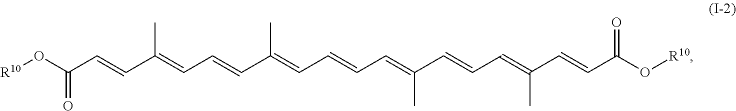
or a geometric isomer thereof, or a combination thereof,
wherein each R¹⁰ is independently selected from hydrogen, a counterion (e.g., a monovalent counterion or a divalent counterion), and an optionally substituted C₁₋₄ alkyl, preferably, both R¹⁰ are the same.

29. The method of claim 28, wherein both R¹⁰ are C₁₋₄ alkyl, preferably, ethyl.

30. The method of claim 28, wherein both R¹⁰ are a monovalent counterion, preferably, an alkali metal ion, such as Li⁺, Na⁺, or K⁺.

31. The method of claim 28, wherein both R¹⁰ are hydrogen.

32. The method of any one of claims 28-31, wherein the aldehyde of formula II has the Formula II-2: 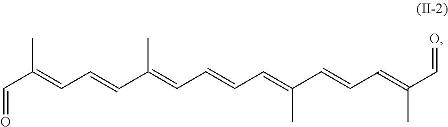 [SMILES: C\C(C=O)=C/C=C/C(C)=C/C=C/C=C(C)/C=C/C=C(\C)C=O]
or a geometric isomer thereof, or a combination thereof.

33. The method of claim 32, wherein the aldehyde of Formula II used for reacting with the olefin forming agent is a substantially pure form of Formula II-2, 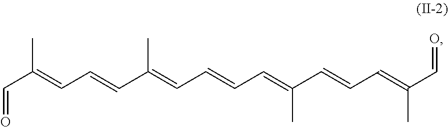 [SMILES: C\C(C=O)=C/C=C/C(C)=C/C=C/C=C(C)/C=C/C=C(\C)C=O]
which includes less than 10% of other geometric isomers by HPLC area percentage with detection wavelength at 254 nm and 420 nm, preferably, less than 5% such as less than 3%, or less than 2% of other geometric isomers.

34. A method of preparing a crocetin having formula I-1: 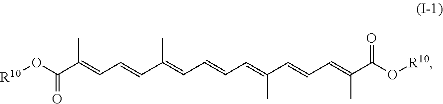
which is in a substantially pure form, e.g., with an applicable purity profile described herein, such as having less than 3% of other geometric isomers by HPLC area percentage with detection wavelength at 254 nm and 420 nm, preferably, less than 2% such as less than 1.5%, or less than 1% of other geometric isomers,
wherein each R¹⁰ is independently selected from hydrogen, a counterion (e.g., a monovalent counterion or a divalent counterion), and an optionally substituted C₁₋₄ alkyl, the method comprising:
a) reacting a dialdehyde of Formula II-1: 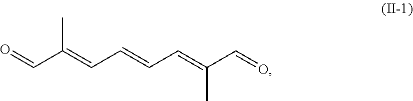
wherein the dialdehyde contains less than 10% of other geometric isomers by HPLC area percentage with detection wavelength at 330 nm, preferably, less than 5% such as less than 3%, or less than 2% of other geometric isomers, with an olefin forming agent of Formula III-3, 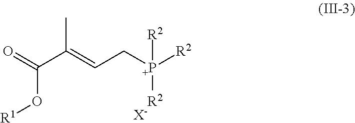
wherein R¹ is an optionally substituted C₁₋₄ alkyl, and each R² is independently an optionally substituted alkyl, an optionally substituted cycloalkyl, or an optionally substituted phenyl, wherein X⁻ is a counterion, e.g., a halide such as chloride or bromide,
to form an intermediate compound of Formula IV-1, 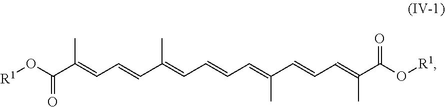
b) isolating the intermediate compound of Formula IV-1 in a substantially pure form, e.g., with an applicable purity profile described herein, such as having less than 3% of other geometric isomers by HPLC area percentage with detection wavelength at 254 nm and 420 nm, preferably, less than 2% such as less than 1.5%, or less than 1% of other geometric isomers, and
c) optionally hydrolyzing the intermediate compound of Formula IV-1 into the compound of Formula I-1 when at least one R¹⁰ is hydrogen or a counterion.

35. The method of claim 34, wherein both R¹ are the same.

36. The method of claim 35, wherein R¹ is not methyl.

37. The method of claim 35, wherein R¹ is ethyl.

38. The method of any one of claims 34-37, wherein both R¹⁰ are the same.

39. The method of claim 38, wherein R¹⁰ is ethyl.

40. The method of claim 38, wherein R¹⁰ is hydrogen.

41. The method of claim 38, wherein R¹⁰ is a monovalent counterion, preferably, an alkali metal ion, such as Li⁺, Na⁺, or K⁺.

42. The method of any one of claims 34-41, wherein the olefin forming agent is a compound of Formula III-3-A: 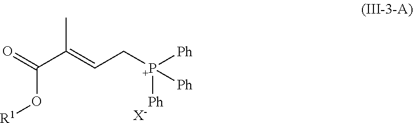
wherein X⁻ is a counterion, e.g., a halide such as chloride or bromide.

43. The method of any one of claims 34-42, wherein the reacting of the dialdehyde and the olefin forming agent is carried out in a non-ether solvent in the presence of a base such as an inorganic base.

44. The method of claim 43, wherein the reacting of the dialdehyde and the olefin forming agent is carried out in a solvent comprising acetonitrile, such as comprising acetonitrile and toluene.

45. The method of claim 43 or 44, wherein the inorganic base is a carbonate base, e.g., sodium carbonate, potassium carbonate, sodium bicarbonate, potassium bicarbonate, etc.

46. The method of any one of claims 43-45, wherein the reacting of the dialdehyde and the olefin forming agent is carried out under heat, e.g., with a reaction temperature ranging from about 50° C.-100° C., or the reaction temperature can be about 45° C. to about 65° C., such as about 60° C., for a first period of time ranging from about 1 hour to about 6 hours, such as about 2, 3, 4, or 5 hours; and subsequently, the reaction temperature can be about 70° C. to about 100° C., such as about 80° C., for a second period of time of ranging from about 10 hours to about 48 hours, such as about 12, 14, 16, 18, 20, or 24 hours.

47. The method of any one of claims 34-46, wherein isolating the intermediate compound of Formula IV-1 comprises cooling the reaction mixture, filtering the reaction mixture, washing the filtered solids with acetonitrile and water, and removing residue phosphine oxide through slurring the washed solids in an alcoholic solvent (e.g., methanol, ethanol, etc.).

48. The method of any one of claims 34-47, wherein the intermediate compound of Formula IV-1 is isolated in a purity of greater than 95%, preferably, greater than 97%, greater than 98%, by HPLC area percentage with detection wavelength at 254 nm and 420 nm.

49. The method of any one of claims 34-48, wherein the dialdehyde of Formula II-1 is prepared by a process comprising isomerizing one or more geometric isomers of Formula II-1, e.g., under an acidic condition, such as with a catalytic amount of phenylsulfinic acid (e.g., generated from sodium phenylsulfinate and HCl), or other acids with similar pKa.

50. The method of any one of claims 34-49, further comprising hydrolyzing the intermediate compound of Formula IV-1, e.g., with an alkali hydroxide, such as LiOH, NaOH, or KOH, to provide the compound of Formula I-1, wherein one or both R¹⁰ is hydrogen or a counterion.

51. The method of any one of claims 34-50, wherein both R¹⁰ are Na⁺.

52. The method of claim 51, further comprising isolating the compound of Formula I-1 from the hydrolysis reaction mixture, e.g., through filtering the solid formed in the reaction mixture, washing the solid with water, washing the solid with an alcoholic solvent (e.g., ethanol), and drying the solid.

53. The method of claim 52, wherein the compound of Formula I-1 is isolated in a substantially pure form, e.g., with an applicable purity profile described herein, such as having a purity of greater than 95%, preferably, greater than 97%, greater than 98%, greater than 99%, by HPLC area percentage with detection wavelength at 254 nm and 420 nm.

54. The carotenoid of Formula I produced by the method of any of claims 1-33.

55. The crocetin of Formula I-1 produced by the method of any of claims 34-53.

56. A crocetin compound of Formula I-1, such as TSC, which is in a substantially pure form, e.g., with an applicable purity profile described herein, such as having a purity of greater than 95%, preferably, greater than 97%, greater than 98%, greater than 99%, by HPLC area percentage with detection wavelength at 254 nm and 420 nm.

57. A method of preparing a pharmaceutical composition comprising formulating the carotenoid of Formula I of claim 54 or the crocetin of Formula I-1 of claim 55 or 56.

58. The method of claim 57, comprising mixing the carotenoid of Formula I of claim 54 or the crocetin of Formula I-1 of claim 55 or 56 with one or more pharmaceutically acceptable excipient or carrier.

59. The method of claim 58, wherein the one or more pharmaceutically acceptable excipient or carrier comprises a polyethylene glycol having a molecular weight of 200-700 Da, e.g., PEG-200, PEG-300, PEG-400, PEG-500, or PEG-600, etc.

60. The method of any of claims 57-59, further comprising
a) mixing the carotenoid of Formula I of claim 54 or the crocetin of Formula I-1 of claim 55 or 56 with one or more pharmaceutically acceptable excipient or carrier with a liposomal solution containing liposomes comprising a weak acid salt of a multivalent metal; and
b) maintaining the mixture of a) for a sufficient time to load the carotenoid or crocetin into liposomes.

61. The method of claim 60, wherein the weak acid is an organic acid (e.g., an organic acid selected from acetic acid, gluconic acid, tartaric acid, glutamic acid, citric acid, formic acid, and glycinic acid).

62. The method of claim 60 or 61, wherein the multivalent metal is a bivalent metal (e.g., a bivalent metal selected from Ca²⁺, Mg²⁺, Zn²⁺, Cu²⁺, Co²⁺, and Fe²⁺), or a trivalent metal such as Fe³⁺.

63. The method of any of claims 60-62, wherein the weak acid is acetic acid and the bivalent metal is Ca²⁺ or Mg²⁺ (i.e., the weak acid salt of the bivalent metal is calcium acetate or magnesium acetate, respectively).

64. The method of any one of claims 60-63, which further comprises the step of heating and cooling of the drug loading mixture.

65. An aqueous solution comprising a liposome prepared according to the method of any one of claims 60-64.

66. A pharmaceutical composition comprising a liposome prepared according to the method of any one of claims 60-64.

67. A pharmaceutical composition comprising the carotenoid of Formula I of claim 54 or the crocetin of Formula I-1 of claim 55 or 56.

68. A pharmaceutical composition produced by the method of any of claims 57-64.

69. The pharmaceutical composition of any of claims 66-68, for use in the treatment or prevention of a disease or a condition in a subject.

70. Use of the pharmaceutical composition of any of claims 66-68, in the manufacture of a medicament for the treatment or prevention of a disease or condition in a subject.

71. A method for treating or preventing a disease or condition in a subject needing such treatment or prevention, the method comprising administering an effective amount of the pharmaceutical composition administering an effective amount of the pharmaceutical composition of any of claims 66-68, to the subject.

72. The pharmaceutical composition of claim 69, use of claim 70, or method of claim 71, wherein the disease or condition is characterized by ischemia (e.g., tissue hypoperfusion) or hypoxia.

73. The pharmaceutical composition, use, or method, of claim 72 wherein the disease or condition characterized by ischemia or hypoxia is tissue hypoperfusion, ischemic-reperfusion injury, transient cerebral ischemia, cerebral ischemia-reperfusion, ischemic stroke, hemorrhagic stroke, traumatic brain injury, migraine (e.g., a chronic migraine or severe migraine disorder), gastrointestinal ischemia, kidney disease, pulmonary embolism, acute respiratory failure, neonatal respiratory distress syndrome, an obstetric emergency to reduce perinatal comorbidity (such as, pre/eclampsia and conditions that lead to cerebral palsy), myocardial infarction, acute limb or mesenteric ischemia, cardiac cirrhosis, chronic peripheral vascular disease, congestive heart failure, atherosclerotic stenosis, anemia, thrombosis, or embolism, wherein the subject has experienced a traumatic injury, or wherein the subject is undergoing or will undergo surgery.

74. The pharmaceutical composition of claim 69, use of claim 70, or method of claim 71, wherein the disease or condition is a lung disease or a condition associated with a lung disease.

75. The pharmaceutical composition, use, or method of claim 74, wherein the disease or condition is pulmonary fibrosis, pulmonary hemorrhage, lung injury, lung cancer, chronic obstructive pulmonary disease (COPD), or another respiratory disorder.

76. The pharmaceutical composition of claim 69, use of claim 70, or method of claim 71, wherein the disease or condition is acute respiratory distress syndrome (ARDS).

77. The pharmaceutical composition of claim 69, use of claim 70, or method of claim 71, wherein the disease or condition is a cardiovascular disease or a condition associated with a cardiovascular disease.

78. The pharmaceutical composition, use, or method of claim 77, wherein the disease or condition is a coronary artery disease such as myocardial infarction, sudden cardiac death, cardiorespiratory arrest, hypertension, pulmonary arterial hypertension, atherosclerosis, Takayasu's arthritis, and post-cardiac arrest syndrome (PCAS), chronic venous insufficiency, heart disease, congestive heart failure, occlusive arterial disease, Raynaud's disease, peripheral vascular disease, or another vasculopathy such as Buerger's disease, or a chronic skin ulcer.

79. The pharmaceutical composition of claim 69, use of claim 70, or method of claim 71, wherein the disease or condition is a heart attack or stroke, or a condition associated with a heart attack or stroke (e.g., an ischemic stroke or hemorrhagic stroke);

80. The pharmaceutical composition of claim 69, use of claim 70, or method of claim 71, wherein the disease or condition is associated with nitric oxide deficiency (e.g., sickle cell disease, paroxysmal nocturnal hemoglobinuria (PNH), a hemolytic anemia, a thalassemia, another red blood cell disorder, a purpura such as thrombotic thrombocytic purpura (TTP), hemolytic uremic syndrome (HUS), idiopathic thrombocytopenia (ITP), another platelet disorder, a coagulation abnormality such as disseminated intravascular coagulopathy (DIC), purpura fulminans, heparin induced thrombocytopenia (HIT), hyperleukocytosis, and or hyper viscosity syndrome).

81. The pharmaceutical composition of claim 69, use of claim 70, or method of claim 71, wherein the disease or condition is shock or a condition associated with shock (e.g., cardiogenic shock, hypovolemic shock, septic shock, neurogenic shock, and anaphylactic shock).

82. The pharmaceutical composition of claim 69, use of claim 70, or method of claim 71, wherein the disease or condition is an infection or a condition associated with an infection (e.g., hypoxia, tissue hypoperfusion, ischemia, ARDS, and sepsis).

83. The pharmaceutical composition, use, or method, of claim 82, wherein the disease or condition is associated with a bacterial infection (e.g., an P. aeruginosa infection, S. aureus infection (e.g., MRSA), or an enterococcal infection (e.g., VRE), such as hypoxia, tissue hypoperfusion, ischemia, and sepsis).

84. The pharmaceutical composition, use, or method, of claim 82, wherein the disease or condition is associated with a viral infection (e.g., hypoxia, tissue hypoperfusion, ischemia, sepsis, and ARDS).

85. The pharmaceutical composition, use, or method, of claim 82, wherein the disease or condition is associated with a coronavirus infection (e.g., COVID-19 and ARDS).

86. The pharmaceutical composition, use, or method, of claim 82, wherein the disease or condition is associated with an Ebola, Dengue or Marburg infection (e.g., influenza, measles, and a viral hemorrhagic fever).

87. The pharmaceutical composition, use, or method, of claim 82, wherein the disease or condition is associated with a fungal infection (e.g., a candidiasis infection such as invasive candidiasis).

88. The pharmaceutical composition, use, or method, of claim 82, wherein the disease or condition is a parasitic infection or a condition associated with a parasitic infection such as malaria (e.g., cerebral malaria, severe anemia, acidosis, acute kidney failure, ischemia, tissue hypoperfusion and ARDS), Schistosomiasis, and human African trypanosomiasis.

89. The pharmaceutical composition of claim 69, use of claim 70, or method of claim 71, wherein the disease or condition is endotoxemia or a condition associated with endotoxemia (e.g., low grade endotoxemic disease, or endotoxemia associated with a condition such as periodontal disease (e.g., periodontitis or inflammation of the gums), chronic alcoholism, chronic smoking, transplantation, neonatal necrotizing enterocolitis, or a neonatal ear infection).

90. The pharmaceutical composition of claim 69, use of claim 70, or method of claim 71, wherein the disease or condition is bacteremia.

91. The pharmaceutical composition of claim 69, use of claim 70, or method of claim 71, wherein the disease or condition is sepsis; or a condition associated with sepsis, or wherein the subject is at risk of developing sepsis or a condition associated with sepsis.

92. The pharmaceutical composition of claim 69, use of claim 70, or method of claim 71, wherein the disease or condition is inflammation or a condition associated with inflammation.

93. The pharmaceutical composition, use, or method of claim 92, wherein the disease or condition is systemic inflammation (e.g., systemic inflammatory response syndrome (SIRS)), low-grade inflammation, acute inflammation, or a chronic inflammatory disease.

94. The pharmaceutical composition of claim 69, use of claim 70, or method of claim 71, wherein the disease or condition is an autoimmune disorder (e.g., psoriasis, cystic fibrosis, and rheumatoid arthritis).

95. The pharmaceutical composition of claim 69, use of claim 70, or method of claim 71, wherein the disease or condition is sclerosis or a condition associated with sclerosis (e.g., systemic sclerosis).

96. The pharmaceutical composition of claim 69, use of claim 70, or method of claim 71, wherein the disease or condition is inflammatory bowel disease or a condition associated with inflammatory bowel disease (e.g., Crohn's disease and ulcerative colitis).

97. The pharmaceutical composition of claim 69, use of claim 70, or method of claim 71, wherein the disease or condition is a metabolic disease or a condition associated with a metabolic disease.

98. The pharmaceutical composition of claim 69, use of claim 70, or method of claim 71, wherein the disease or condition is insulin resistance or a condition associated with insulin resistance.

99. The pharmaceutical composition of claim 69, use of claim 70, or method of claim 71, wherein the disease or condition is diabetes or a condition associated with diabetes such as gangrene, diabetic necrosis, diabetic neuropathy, diabetic vascular disease (e.g., microvascular disease such as retinopathy and nephropathy, and diabetic ulcers).

100. The pharmaceutical composition of claim 69, use of claim 70, or method of claim 71, wherein the disease or condition is type 2 diabetes or a condition associated with type 2 diabetes.

101. The pharmaceutical composition of claim 69, use of claim 70, or method of claim 71, wherein the disease or condition is liver disease or a condition associated with a liver disease (e.g., cirrhosis, nonalcoholic fatty liver disease (NAFLD), non-alcoholic steatohepatitis (NASH); alcoholic liver disease, acute liver injury, and cirrhosis of the liver).

102. The pharmaceutical composition of claim 69, use of claim 70, or method of claim 71, wherein the disease or condition is kidney disease or a condition associated with kidney disease (e.g., lipopolysaccharide medication or toxin induced acute kidney injury (AKI) and end stage kidney disease).

103. Use of the pharmaceutical composition of any of claims 66-68 in the manufacture of a medicament for increasing the delivery of oxygen to the cells in a subject.

104. A method of increasing the delivery of oxygen to the cells and tissues in a subject, the method comprising administering an effective amount of the pharmaceutical composition of any of claims 66-68 to the subject.

105. The use of claim 103 or method of claim 104, wherein the subject is in need of increased oxygen delivery.

106. The use of claim 103 or method of claim 104, wherein the pharmaceutical composition is administered to increase the physical or mental performance of the subject.

107. Use of the pharmaceutical composition of any of claims 66-68 in the manufacture of a medicament for reducing systemic levels of LPS, endotoxin and/or another trigger of systemic inflammation in a subject.

108. A method of reducing systemic levels of LPS, endotoxin and/or another trigger of systemic inflammation in a subject in need thereof, the method comprising administering an effective amount of the pharmaceutical composition any of claims 66-68 to the subject.

109. The use or method according to any one of claims 69-108, wherein the age of the subject is 50 or older, 55 or older, 60 or older, 65 or older, 70 or older, 75 or older, or 80 or older.

110. The use or method according to any one of claims 69-109, wherein the subject is immunocompromised.

111. The use or method according to any one of claims 69-110, wherein the subject receives chemotherapy and/or is immune-suppressed (e.g., febrile neutropenic patients).

112. The use or method according to any one of claims 69-111, wherein the subject is a burn victim.

113. The use or method according to any one of claims 69-112, wherein the subject is critically ill.

114. The use or method according to any one of claims 69-113, wherein the pharmaceutical composition is administered in combination therapy with another therapeutic agent.

115. The use or method according to any one of claims 69-114, wherein subject is a human.
